(19) United States
(12) Patent Application Publication
(10) Pub. No.: US 2026/0273059 A1
(43) Pub. Date: Sep. 17, 2026

(54) METHODS OF EVADING IMMUNE REJECTION

(71) Applicant: William Marsh Rice University, Houston, TX (US)

(72) Inventors: Omid VEISEH, Houston, TX (US); Amanda NASH, Houston, TX (US); Boram KIM, Houston, TX (US); Dilrasbonu VOHIDOVA, Houston, TX (US); Samantha FLEURY, Houston, TX (US)

(21) Appl. No.: 19/469,418

(22) PCT Filed: Mar. 28, 2024
(86) PCT No.: PCT/US24/21949

§ 371 Date: Sep. 26, 2025

Related U.S. Application Data
(60) Provisional application No. 63/455,050, filed on Mar. 28, 2023.

(51) Int. Cl. A61K 40/35; A61K 9/00; A61K 45/06

(52) U.S. Cl. CPC A61K 40/35; A61K 9/0024; A61K 45/06

(57) ABSTRACT

The present disclosure is directed to methods, compositions and devices capable of avoiding foreign body responses to implanted material. This technology utilizes polymer encapsulated engineered cells that produce natural, cell-generated cytokines to elicit a tailored immune response to implanted materials for applications in diabetes, immune tolerance, and organ transplantation.

### PRIORITY CLAIM

This application claims benefit of priority to U.S. Provisional Application Ser. No. 63/455,050, filed Mar. 28, 2023, the entire contents of which are hereby incorporated by reference.

### REFERENCE TO A SEQUENCE LISTING

The application contains a sequence listing in computer readable form, which has been submitted electronically and is hereby incorporated by reference in its entirety. The Sequence Listing created on Mar. 28, 2024, is named RICEP0128WO.xml and is 864,942 bytes in size.

### BACKGROUND

### Field of the Disclosure

The present disclosure relates generally to the fields of immunology, medicine and transplant biology. More particular, the disclosure relates to development of implantable materials that employ encapsulated engineered cells that product immune-modulating cytokine that prevent destruction of the implanted materials. It further relates to the fibrosis of implanted materials.

### Background

Advances in biomedical research have led to methods for localized and targeted therapies for the treatment of diseases, such as diabetes. However, in many instances, the percentage of patients responsive to these approaches remain modest.

One approach involves the use of implantable devices to deliver therapeutic agents. A fundamental barrier to successful device-based therapies is the lack of biocompatible transplantation devices. Implanted biomaterials lead to foreign body responses (FBRs), which are cascades of inflammatory events and wound-healing processes that lead to fibrosis (walling off) and subsequent implant failure. This response has been described for many materials, from naturally occurring polymers to synthetic materials. Thus, a critical medical need exists to develop biomaterials that overcome this key challenge of eliminating the FBR associated with biomaterial grafting. In addition, there is a need to control (i.e., down-regulate or stop) therapies after a period of time. At present, techniques for achieving the dual goals of FBR and controlled delivery of therapeutic agents are lacking.

Thus, there is a need for identifying new compositions and methods to enhance the delivery, distribution, and/or efficacy of therapeutic agents.

### SUMMARY

Thus, in accordance with the present disclosure, an implantable construct comprising an engineered cell expressing one or more cytokines, such as interleukin-12 (IL-12), interleukin-10 (IL-10) and/or interleukin-35 (IL-35). The expression of said one or more cytokines may be continuous, optionally further regulatable, such downregulated by an inducible “off” control. The construct may further comprise a therapeutic agent, such as a DNA, RNA, or an oligonucleotide, a further modulator of a host immune system, an organo-pharmaceutical compound, a toxin, or a protein (other than IL-12, IL-10 or IL-35). The construct may provide sustained or pulsatile release of the therapeutic agent.

The engineered cell may be a Chinese hamster ovary (CHO) cell, a retinal pigment epithelial cell (ARPE-19), a human mammary epithelial cell (MCF-10a and MCF-7), a human embryonic kidney (HEK) cell, a mesenchymal stem cell (MSC), a human umbilical vein endothelial cell (HUVEC), an NIH/3T3 cell, a BJ fibroblast cell, or a human renal mix epithelial cell (HREC). The therapeutic agent may be released over at least 1 hour to 30 days, 60 day, 90 days, 180 days or 1 year. The construct may comprise a polymeric hydrogel, such as a polymeric hydrogel comprising chitosan, cellulose, hyaluronic acid, or alginate.

Also provided is a method of providing an implantable construct to a subject comprising implanting into to the subject, or providing the subject with, an implantable construct as described here.

Additionally, there is provided as method of making or manufacturing an implantable construct comprising introducing into an implantable construct an engineered cell expressing one or more cytokines, such as interleukin-12 (IL-12), interleukin-10 (IL-10) and/or interleukin-35 (IL-35). The expression of said one or more cytokines may be regulatable, such as by an inducible “off” control. The construct may further comprise a therapeutic agent. The engineered cell may be a Chinese hamster ovary (CHO) cell, a retinal pigment epithelial cell (ARPE-19), a human mammary epithelial cell (MCF-10a and MCF-7), a human embryonic kidney (HEK) cell, a mesenchymal stem cell (MSC), a human umbilical vein endothelial cell (HUVEC), an NIH/3T3 cell, a BJ fibroblast cell, or a human renal mix epithelial cell (HREC). The construct may comprise a polymeric hydrogel, such as chitosan, cellulose, hyaluronic acid, or alginate.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B. (FIG. 1A) Preimplant images of mIL10-RPE (1.5 mm), and human islet (0.5 mm) implantable constructs (live: triangle/dead: square). FIG. 1B. Blood glucose monitoring after implantable construct transplantation.

FIGS. 2A-B are schematics illustrating a system comprised of hydrogel-based implantable constructs, i.e., capsules, that evade activation of this immune response through delivery of a therapeutic agent, e.g., heterodimeric IL-12. FIG. 2A is a schematic illustrating that hydrogels are naturally recognized as foreign by immune cells when introduced to the body and subsequently coated with fibrotic deposition as a method of protection for animals. FIG. 2B is a schematic depicting a system comprised of hydrogel-based capsules that evade activation of this immune response through delivery of heterodimeric IL-12. One subunit, p35, is anti-inflammatory while the other subunit, p40, is pro-inflammatory. Together, these subunits comprise the proinflammatory cytokines IL-12. IL-12 prevents the materials from being coated by the host immune response, thus allowing continuous local delivery of the intended therapeutic. This system is customizable by simply spiking IL-12 engineered cells into capsules containing other cytokines of interest. This allows for controlled and predictable modulation of the immune system.

FIG. 3 shows representative lead modified alginate structures.

FIG. 4 is a series of images with 50 implantable constructs, i.e., capsules, after 30 days. Variation of the implantable cytokine factories will consist of at least 80% cell of interest and 20% RPE-mIL12 cells. These capsules will remain functional at the desired implant site for at least 30 days without becoming fibrosed by the cells of the host immune system. Mixing cells does not affect cell viability as indicated by the green color. The cell of interest will be any of the cells listed herein, genetically modified to express any of the cytokines listed herein. This will allow for optimal modularity of the system for treatment of a wide range of disease implications.

FIGS. 5A-D show the results of mIL12- and mIL10-producing RPE cells encapsulated with 1.5 mm alginate capsules. FIG. 5A is a schematic showing that empty alginate microcapsules (0.5 mm diameter) were fabricated to co-deliver with the 1.5 mm capsules encapsulated cells Since smaller sized microcapsules have been reported to produce rapid immune responses. Non-engineered RPE cells were used as control. FIG. 5B are a series of images showing that each cell line was encapsulated at a density of 10,000 cells per capsule, and a total of 10 capsules of either mIL12 or mIL10 capsules were implanted in the IP space together with 0.4 mL of microcapsules. FIG. 5C are micrographs demonstrating that after 4 weeks, all capsules were retrieved and observed under a microscope to visualize fibrosis on the capsule surface (FIG. 5C) Both mIL12 (FIG. 5C1) and mIL10 (FIG. 5C2) groups showed minimal fibrosis deposition onto microcapsules, while the RPE group (FIG. 5C3), had highly packed fibrosis in both large RPE capsules and empty microcapsules. FIG. 5D is a bar graph showing that these groups had lower expressions of fibrotic markers (aSMA and Col1a1) from RT-qPCR analysis. This result showed the antifibrotic effect of mIL12- and mIL10-producing cells upon implantation in healthy mice.

FIGS. 6A-C demonstrate that localized cytokine production from encapsulated cells can prevent fibrosis of implants in healthy mice. FIG. 6A is a series of images demonstrating an approach to co-deliver human islets in diabetic mice. mIL12-RPE cells were encapsulated with SLG20 alginate at 10,000 cells/capsule density with 1.5 mm diameter. Non-engineered RPE capsules at the same density were used as a control. 2,000 IEQ of human islets were encapsulated with 0.4 mL of SLG20 (FIG. 6A). FIG. 6B illustrates that islet capsules were fabricated in 0.5 mm diameter 10 1.5 mm size capsules encapsulating mIL12-RPE cells and 0.4 mL of islet-microcapsules were implanted into STZ-induced diabetic mice. FIG. 6C is a graph showing that RPE capsules failed to regulate blood glucose levels after 3 weeks, while mIL12-RPE group restored diabetic mice to normoglycemia.

FIGS. 7A-I demonstrate that the lead hydrogel from the hydrogel screen enables diabetic reversal in immunocompetent C57BL/6J mice when used to encapsulate human islets. FIG. 7A us a series of representative images of pre-implant capsules. Z4-A10 capsules containing human islets at a density of 10 IEQ/capsule, 20 IEQ/capsule, and 40 IEQ/capsule, respectively. Z1-A34 and SLG20 capsule was used as control material. Dithizone staining indicates viable islets within the capsule matrix. After encapsulation, islets show good viability (live: green, dead: red). FIG. 7B is a graph showing that blood Glucose levels for both Z4-A10 and SLG20 groups (4,000 IEQ/mL density) were monitored until mice were euthanized (**** P<0.0001 (SLG20 vs. Z4-A10)). FIG. 7C is a bar graph showing that an IVGTT test with Z4-A10 capsule (4,000 IEQ/mL) implant group in diabetic mouse, an non-implant group in diabetic mice and non-diabetic mice (ns; not significant, **** P<0.0001 (all comparisons)). FIGS. 7D-E are representative dark-field (FIG. 7D), and dithizone staining (red, FIG. 7E) images of explanted Z4-A10 and SLG20 capsules (4,000 IEQ/mL). FIG. 7F is a bar graph showing human c-peptide measurements at 80-days post-transplantation (SLG20 vs. Z4-A10). FIGS. 7G-I are graphs showing blood glucose monitoring with high islets density groups: Z4-A10 capsules (FIG. 7G), Z1-A34 capsules (FIG. 7H), and SLG capsules (FIG. 7I). Error bars denote mean±sem; Two-way ANOVA with Bonferroni multiple comparison correction.

FIG. 8. demonstrates that cytokine producing, kill switch inducible cell line responds to 100 nM AP1903 in less than 24 hours as shown in the images (left and middle). The cells which were not subjected to the small molecule kill switch inducer remained highly viable, thus allowing for sustained production of the IL-2 protein as shown in the bar graph (right). This will allow for increased safety for all future iterations of the inventors' technology.

FIG. 9. IFN-g can also be used as a reporter for toxicity. Following an increase in the local mIL12 cytokine levels, the inventors saw an increase in local IFN-g concentration that can be utilized as a marker for regulating the production of cytokines from the biomaterial system. Concentration of A) mIL-12 and B) mIFN-γ in the IP-fluid 1-, 4-, and 7-days after RPE-mIL12 administration or sham control.

FIGS. 10A-C. FIG. 10A: top: fibrosis progression schematic of alginate encapsulated islets highlighting immune cells and cytokines involved. FBR is initiated by protein absorption, marking the early stage of inflammation, followed by the infiltration of granulocytes and monocytes to the implantation site. Differentiated macrophages from infiltrating monocytes secrete IL-1, IL-8, CXCL13, monocyte chemotactic protein-1 (MCP-1), and macrophage inflammatory protein (MIP), leading to the recruitment and activation of macrophages at the implant site. Attempting to phagocytose the implant, macrophages at the implant site fuse to create FBGCs. Meanwhile, essential factors for FBGC formation, such as transforming growth factor-β (TGF-β), IL-1, and TNF-α, are present in high concentrations near the implant site. Additionally, TGF-β induces the transformation of fibroblasts into myofibroblasts, and the number of α-smooth muscle actin (aSMA) positive cells increases over time, indicating the onset of fibrosis. Bottom: schematic demonstrating that retinal pigmented epithelial (RPE) cells can be engineered to produce the cytokine of interest for local immodulation, IL-10 can inihibit myeloid cells by inihibitingpro-inflammatory cytokines and antigen-presenting cells (APCSs), IL-12 leads to macrophage polarization, promoting pharogcytosis to slow down fibrosis, and IL-2mt possesses a reduced affinity for the IL-2Rβγ receptor, thereby resulting in selective production of T_{reg} cells. FIG. 10B is a set of microscopy images showing both dark-field (top) and live-dead (bottom) images of implantable constructs comprising cytokine-secreting cells. Scale bar: 2 mm. FIG. 10C is a set of bar graphs showing cytokine levels produced by implantable constructs prior to implantation after 24 h of incubation as measured by ELISA for the RPE-IL10, RPE-IL12 and RPE-IL-2mt implantable constructs.

FIGS. 11A-E demonstrates that co-delivery of cytokine-producing implantable constructs inhibits fibrosis progression in a rodent model over at least a short duration. FIG. 11A is a schematic of local cytokine delivery in the intraperitoneal space of a C57BL/6J mouse for one month. FIG. 11B is a series of bar graphs illustrating cytokine production in local (i.e., intraperitoneal) and systemic (i.e., blood plasma) levels after 1-month post-implantations of implantable constructs comprising RPE cells capable of secreting IL-10, IL-12, and IL-2mt. FIG. 11C shows a series of exemplary dark-field microscopy images of pre-implanted and explanted implantable constructs. FIG. 11D is a series of immunofluorescence images of explanted implantable constructs. Scale bar, 200 μm. FIG. 11E shows bar graphs of an RT-qPCR analysis comparing fibrotic gene expression patterns for αSMA, Col1a1, ad Col1a2 normalized to empty implantable construct control. All graphs are mean±S.E.M> of biological replicates (n=6). Twp-way ANOVA with Bonferroni correction was used for statistical analysis (**** p<0.0001, *** p<0.0002, ** p<0.002, *p<0.033).

FIG. 12 is a series of dark-filed microscopy images derived from an individual mouse specimen one month after intraperitoneal implantation with sham, empty microcapsules, implantable constructs comprising nonengineered RPE cells (RPE), implantable constructs comprising RPE cells secreting IL-10 (RPE-IL10), implantable constructs comprising RPE cells secreting IL-12 (RPE-IL12), implantable constructs comprising RPE cells secreting IL-2mt (RPE-IL2mt), and implantable constructs comprising RPE cells secreting TGF-β.

FIGS. 13A-B demonstrate long-term effects of RPE-IL10 in preventing fibrosis. FIG. 13A shows dark-field microscopy images of explanted implantable constructs comprising RPE cells capable of secreting IL10 (RPE-IL10) and empty microcapsules retrieved three months after IP implantation in a rodent model. FIG. 13B shows dark-field microscopy images of explanted implantable constructs comprising RPE-IL10 and empty microcapsules retrieved six months after IP implantation in a rodent model.

FIGS. 14A-B demonstrate the long-term effects of RPE-1L12 in preventing fibrosis. FIG. 14A shows dark-field microscopy images of explanted implantable constructs comprising RPE cells capable of secreting IL12 (RPE-IL12) retrieved three months after IP implantation in a rodent model. FIG. 14B shows dark-field microscopy images of explanted implantable constructs comprising RPE-IL12 retrieved six months after IP implantation in a rodent model.

FIG. 15 shows graphs of cytokine concentration (left: RPE-IL10 implant group; right: RPE-IL12 implant group) in IP fluid relative to the bloodstream over 180 days.

FIGS. 16A-E demonstrate that IL-10 prevents fibrosis by suppressing imflammation, whereas IL-12 inhibits fibrosis by inhibiting the TGFβ pathway. FIG. 16A left: shows a UMAP (left) embedding of individual cells pooled from all samples. Resulting clusters are classified by immune cell type on the basis of cell-specific expression profiles and the composition (right) of pericapsular immune cell identities recruited to each implant site; middle: is a bar graph showing the composition of local immune cell identities recruited to each implant site; right: Violin plots showing the TGFβ score in monocytes. FIG. 16B is heatmap showing z-scaled expression levels of representative genes from the inflammatory and TGFβ pathways. FIG. 16C left, middle: Volcano plots showing the differential expression in monocytes treated with RPE-IL10 (left) and RPE-IL12 (right), compared to RPE alone; right: violin plot showing the inflammatory cytokine Cxc19 in monocyte with RPE-IL10, RPE, and RPE-IL12 (p=2.9e-16 for RPE-IL10 or RPE-IL12 vs. RPE). FIG. 16D: map showing the expression levels of Tgfb2 and Cxc19 in intraperitoneal immune cells with RPE-IL10, RPE and RPE-IL12. FIG. 16E: schematic representing how RPE-IL10 and RPE-IL12 mediated fibrosis suppression.

FIGS. 17A-C demonstrate the effect of cytokines in immune cells. FIG. 17A: single-cell transcriptional profiles of markers used to define each immune cell type in intraperitoneal cells. FIG. 17B: bar graph displaying composition of splenic immune cells. FIG. 17C: single cell transcriptional profiles of markers used to define each immune cell type in splenic cells.

FIGS. 18A-G demonstrate that RPE-IL10 implantable constructs co-delivered with encapsulated xenogeneic human islets achieves glucose correction in immunocompetent STZ-induced diabetic C57BL/6J mice. FIG. 18A is a series of representative images of implantable constructs prior to implantation. Dark-field microscopy images are shown with non-engineered RPE cells (RPE), RPE cells capable of secreting IL-10 (RPE-IL10), and RPE cells capable of secreting IL-12 (RPE-IL12) in 1.5 mm-diameter. After encapsulation, the RPE islets demonstrate good viability as assessed by LIVE-DEAD cell assay. Dithizone staining (in red) shows B-cells inside the 0.5 mm diameter implantable constructs. FIG. 18B illustrates implantation of cytokine-secreting RPE implantable constructs in conjunction with encapsulated human islets in the intraperitoneal space of STZ-induced diabetic mice. FIG. 18C shows graphs of blood glucose (BG) levels of RPE, RPE-IL-10, and RPE-IL-12 groups delivered with encapsulated IEQ islets (2,000 cells) for 50 days (n=6). Mice which have BG levels below the dashed line (250 mg/dL) are considered to be in normoglycemic condition. All error bars denote mean±S.E.M. of the biological replicates. FIG. 18D is a graph illustrating BG levels of RPE and RPE-IL10 implantable constructs further comprising islets, in addition to implantable constructs containing islets alone for 100 days (n=6). All error bars denote mean±S.E.M. biological replicates. FIG. 18E is a bar graph showing the local (i.e., in the IP space) IL-10 concentration and systemic (i.e., blood plasma) concentration from the RPE-IL-10 group at day 100. FIG. 18F are representative dark field microscopy images of retrieved implantable constructs containing islets alone, RPE cells, and RPE cells capable of secreting IL-10 100 days after implantation. FIG. 18G is a bar graph showing human c-peptide levels in serum at day 100 post transplantation for implantable constructs containing islets alone, RPE cells, and RPE cells capable of secreting IL-10. One-way ANOVA with Bonferroni correction was used for statistical analysis (*P<0.033).

FIG. 19A-C. demonstrate that RPE-IL10 implantable constructs prevent diabetes onset in a NOD mice model. FIG. 19A shows the experimental design for the implantation of RPE-IL10 implantable constructs into the IP space of 8-week-old NOD/ShiLtJ mice (top), and incidence of spontaneous diabetes in Sham (saline injection, n=7), RPE (n=10), and RPE-IL-10 (n=10) groups monitored over a period of 24 weeks until euthanasia. FIG. 19B shows graphs of individual BG levels for Sham, RPE, and RPE-IL10 groups for 24 weeks. Some of the mice from the Sham and RPE groups reached human endpoint before 24 weeks, becoming fully diabetic. FIG. 19C shows representative histology images taken in brightfield setting at 20× magnification of mouse pancreases from healthy immunocompetent C57BL/6J (B6) mice, Sham, RPE, RPE-IL10 groups, indicating the presence of insulitis at each condition (left); and insulitis score graph for Sham, RPE, and RPE-IL-10 groups. Islets for each mouse were score and the percentage of each scoring per mouse was calculated and an average was plotted of each score as a final score of insulitis.

FIG. 20 show representative examples of insulitis scores and percentages of insulitis scores in individual mice. Top: Representative images of murine pancreas taken in bright-field setting at 20× magnification, illustrating islet insulin scoring: 0: no insulitis (i.e., no lymphocyte infiltration); 1: peri-insulitis (lymphocytes surrounding the islet); 2:25-50% lymphocytes infiltration; 3:50-70% lymphocyte infiltration; and 4: nearly complete to complete lymphocyte infiltration and destruction of the islets. Lymphocyte infiltration is indicated via the white arrows. Bottom: A heatmap depicting the percentage of insulitis scores of individual mice. Percentage from total islets for each scoring per mouse is shown.

FIG. 21 demonstrates validation of cellular IL-35 production by ELISA, measuring the production rate of RPE cells transfected by IL-35 expression plasmids. Left: a schematic of IL-35 expression plasmids. CAG: CMV enhancer; EBI3: Epstein-Barr virus induced gene 3 (a subunit of IL-35); GGGS) 3: glycine/serine flexible linker; p35: subunit of IL-35; IEGRMD: linker peptide; mIgG2a: Fc region; IRES: internal ribosome entry site; P2A: self-cleaving peptide. Right: A graph showing RPE cells transfected with IL-35 vectors with the media changes 24 h after transfection. IL-35 concentration was assayed by ELISA 24 later.

FIG. 22 demonstrates that encapsulated IL35-Flexi-Fc cells delay onset of diabetes in a mildly-STZ induced diabetic mouse model. Left: Fluorescence microscopy images of IL35-Flexi-Fc cells assayed by LIVE/DEAD demonstrate high viability after encapsulation. Middle: A bar graph of IL-35 concentration determined by ELISA from implantable constructs prior to implantation. Right: A graph showing C57BL/6J mice that were daily administered 40 mg/kg of STZ via IP injection for five days; on the sixth day, the IL35-Flexi-Fc implantable constructs were implanted in the IP space of the mice. BG measurements were taken every other day, beginning on the ninth day. RPE group serves as a negative control. Dashed line indicates mice that are normoglycemic (<250 mg/dL). RPE group (n=2); IL35-Flex-Fc (n=3).

FIG. 23 demonstrates validation of cellular IL-12p35 production. ELISAs were performed to measure the production rate of RPE cells transfected by IL-12p35 expression plasmids. Left: A schematic of IL12p35-Fc and IL-12p35 with hIL2 signaling sequence plasmids. CAG: CMV enhancer; IRES: internal ribosome entry site; IEGRMD: linker peptide; mIgG2a: Fc region. Right: A bar graph showing IL-12p35 concentration (pg/day) of RPE cells transfected with IL12p35-Fc and IL12-p35 with hIL2 signaling vectors with the media changed 24 h after transfections. IL-12p35 concentration in media was determined by ELISA 24 h later (n=3).

FIG. 24 demonstrates validation of cellular IL-23 and IL-27 production. The cytokine concentrations were determined by ELISA from the production rate of RPE cells transfected by IL-23 and IL-27 expression patterns. Left: A schematic of IL-23 and IL-27 expression plasmids. CAG: CMV enhancer; IRES: internal ribosome entry site. Right: Bar graphs showing cytokine concentration (pg/day) from RPE cells transfected with IL-27 and IL-23 vectors, respectively, with the media changed 24 h post-transfection. IL-27 and IL-23 concentrations in media were determined by ELISA 24 h later.

### DESCRIPTION OF ILLUSTRATIVE EMBODIMENTS

As discussed above, there remain significant limitations on the use of implantable materials to intervene in a variety of clinical scenarios, such as to provide therapeutic agents in subjects over an extended time period. Here, the inventors report a new approach to inhibiting the foreign body response to implanted material. Using polymer encapsulated engineered cells (e.g., that produce natural, cell-generated cytokines to modify innate immune response to implanted materials), it is now possible to provide more effective, long-term therapies to conditions such as diabetes, immune tolerance, and organ transplantation. The engineered cells, in one aspect, deliver interleukin-12 (IL-12). While limited by the overall pro-inflammatory nature of IL-12, only a very small dose is needed to illicit the desired effects and, moreover, the encapsulated cells are programmed to permit careful control over-dosing, thereby avoiding any toxicities.

These and other aspects of the disclosure are described in detail below.

### Definitions

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

“Antigenic agent,” as used herein, is a substance which induces, activates, or evokes an immune response, e.g., in a subject.

“Cell,” as used herein, refers to an individual cell. In an embodiment, a cell is a primary cell or is derived from a cell culture. In an embodiment, a cell is a stem cell or is derived from a stem cell. A cell may be xenogeneic, autologous, or allogeneic. In an embodiment, a cell is be engineered (e.g., genetically engineered) or is not engineered (e.g., not genetically engineered).

“Degradable,” as used herein, refers to a structure which upon modulation, e.g., cleavage, decreases the ability of the zone of the implantable construct (e.g., the inner zone and/or the outer zone) to impede contact of a host immune effector with the zone (e.g., the inner zone and/or the outer zone) or a component disposed in the zone. For example, the degradable entity can comprise a site which is cleavable by an enzyme, e.g., an endogenous host enzyme, or an administered enzyme. Typically, the degradable entity mediates a physical property of a zone, e.g., the inner zone or the outer zone, for example, the thickness, degree of cross-linking, or permeability, which impedes passage of a host agent (e.g., a host immune component, e.g., a host immune cell).

“Prevention,” “prevent,” and “preventing” as used herein refers to a treatment that comprises administering or applying a therapy, e.g., administering an implantable construct (e.g., as described herein) comprising a therapeutic agent (e.g., a therapeutic agent described herein) prior to the onset of a disease or condition in order to preclude the physical manifestation of said disease or condition. In some embodiments, “prevention,” “prevent,” and “preventing” require that signs or symptoms of the disease or condition have not yet developed or have not yet been observed. In some embodiments, treatment comprises prevention and in other embodiments it does not.

“Subject,” as used herein, refers to the recipient of the implantable construct described herein. The subject may include a human and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development (e.g., a male or female of any age group, e.g., a pediatric subject (e.g., infant, child, adolescent) or adult subject (e.g., young adult, middle-aged adult, or senior adult). A non-human animal may be a transgenic animal.

“Treatment,” “treat,” and “treating,” as used herein, refer to reversing, alleviating, delaying the onset of, or inhibiting the progress of one or more of a symptom, manifestation, or underlying cause of a disease or condition. (e.g., as described herein), e.g., by administering or applying a therapy, e.g., administering an implantable construct comprising a therapeutic agent (e.g., a therapeutic agent described herein). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease or condition. In an embodiment, treating comprises reducing, reversing, alleviating, reducing, or delaying the onset of, an underlying cause of a disease or condition. In some embodiments, “treatment,” “treat,” and “treating” require that signs or symptoms of the disease or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

### Chemical Definitions

Definitions of specific functional groups are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The chemical structures and formulae set forth herein are constructed according to the standard rules of chemical valency known in the chemical and biological arts. Also, all publications, patent applications, patents and other references mentioned herein are incorporated by reference in their entirety.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The compounds disclosed herein may possess one or more chiral centers and so exist in a number of stereoisomeric forms. All stereoisomers and mixtures thereof are included in the scope of the present disclosure. Racemic compounds may either be separated using preparative HPLC and a column with a chiral stationary phase or resolved to yield individual enantiomers utilizing methods known to those skilled in the art. In addition, chiral intermediate compounds may be resolved and used to prepare chiral compounds of the disclosure.

The compounds disclosed herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

“Alkyl” refers to a hydrocarbon group containing one or more carbon atoms, where multiple carbon atoms if present are joined by single bonds. The alkyl hydrocarbon group may be straight-chain or contain one or more branches or cyclic groups having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

“Haloalkyl” refers to an alkyl, wherein one or more hydrogen atoms of the hydrocarbon group are replaced with a halogen, i.e., fluorine, chlorine, bromine, and iodine. “Fluoroalkyl” refers to an alkyl, wherein one or more hydrogen atoms of the hydrocarbon group are replaced with a fluorine. “Chloroalkyl” refers to an alkyl, wherein one or more hydrogen atoms of the hydrocarbon group are replaced with a chlorine. “Bromoalkyl” refers to an alkyl, wherein one or more hydrogen atoms of the hydrocarbon group are replaced with a bromine. “Iodoalkyl” refers to an alkyl, wherein one or more hydrogen atoms of the hydrocarbon group are replaced with a iodine.

The term “halo” encompasses fluoro, chloro, bromo, and iodo.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 12 carbon atoms (“C₂-C₁₂ alkenyl”), 2 to 10 carbon atoms (“C₂-C₁₀ alkenyl”), 2 to 8 carbon atoms (“C₂-C₈ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂-4 alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 12 carbon atoms (“C₂-C₁₀ alkynyl”), 2 to 10 carbon atoms (“C₂-C₁₀ alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃) 3. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5] decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

As used herein, “hydroxy” refers to the radical-OH.

Alkyl, alkenyl, alkynyl, heteroalkyl, cycloalkyl, and heterocyclyl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

### Cytokines for Engineered Cell Expression

### Interleukin-10

Interleukin 10 (IL-10), also known as human cytokine synthesis inhibitory factor (CSIF), is an anti-inflammatory cytokine. In humans, interleukin 10 is encoded by the IL10 gene. IL-10 signals through a receptor complex consisting of two IL-10 receptor-1 and two IL-10 receptor-2 proteins. Consequently, the functional receptor consists of four IL-10 receptor molecules. IL-10 binding induces STAT3 signalling via the phosphorylation of the cytoplasmic tails of IL-10 receptor 1+IL-10 receptor 2 by JAK1 and Tyk2 respectively. The IL-10 protein is a homodimer; each of its subunits is 178-amino-acid long.

IL-10 is classified as a class-2 cytokine, a set of cytokines including IL-19, IL-20, IL-22, IL-24 (Mda-7), IL-26 and interferons type-I (IFN-alpha, -beta, -epsilon, -kappa, -omega), type-II (IFN-gamma) and type-III (IFN-lambda, also known as IL-28A, IL-28B, and IL-29).

In humans, IL-10 is encoded by the IL10 gene, which is located on chromosome 1 and comprises 5 exons, and is primarily produced by monocytes and, to a lesser extent, lymphocytes, namely type-II T helper cells (T_{H}2), mast cells, CD4⁺CD25⁺Foxp3⁺ regulatory T cells, and in a certain subset of activated T cells and B cells. IL-10 can be produced by monocytes upon PD-1 triggering in these cells. IL-10 upregulation is also mediated by GPCRs, such as beta-2 adrenergic and type 2 cannabinoid receptors. The expression of IL-10 is minimal in unstimulated tissues and seems to require triggering by commensal or pathogenic flora. IL-10 expression is tightly regulated at the transcriptional and post-transcriptional level. Extensive IL-10 locus remodeling is observed in monocytes upon stimulation of TLR or Fc receptor pathways. IL-10 induction involves ERK1/2, p38 and NF-κB signalling and transcriptional activation via promoter binding of the transcription factors NF-κB and AP-1. IL-10 may autoregulate its expression via a negative feed-back loop involving autocrine stimulation of the IL-10 receptor and inhibition of the p38 signaling pathway. Additionally, IL-10 expression is extensively regulated at the post-transcriptional level, which may involve control of mRNA stability via AU-rich elements and by microRNAs such as let-7 or miR-106.

IL-10 is a cytokine with multiple, pleiotropic, effects in immunoregulation and inflammation. It downregulates the expression of Th1 cytokines, MHC class II antigens, and co-stimulatory molecules on macrophages. It also enhances B cell survival, proliferation, and antibody production. IL-10 can block NF-κB activity and is involved in the regulation of the JAK-STAT signaling pathway.

Discovered in 1991, IL-10 was initially reported to suppress cytokine secretion, antigen presentation and CD4+ T cell activation. Further investigation has shown that IL-10 predominantly inhibits lipopolysaccharide (LPS) and bacterial product mediated induction of the pro-inflammatory cytokines TNFα, IL-1β, IL-12, and IFNγ secretion from Toll-Like Receptor (TLR) triggered myeloid lineage cells.

### Interleukin-12 (IL-12) Family of Cytokines

The engineered cells are contemplated to deliver a therapeutic agent comprising a protein such as a cytokine. In an embodiment, the cytokine belongs to the interleukin-12 (IL-12) family of cytokines, e.g., IL-12, IL-23, IL-27 and IL-35. The IL-12 family is the only group of interleukins comprised of heterodimers of modular protein subunits which are shared among its cytokines, cytokine receptors, and secondary messenger molecules, e.g., JAK and STAT signaling partners. IL-12 is a heterodimer of p35 and p40 subunits; IL-23 is a heterodimer of p19 and p40 subunits; IL-27 is a heterodimer of p28 and EBI3 subunits; and IL-35 is a heterodimer p35 and EBI3. The modularity of this set of four protein subunits allows, in part, for the IL-12 family to exert diverse immunomodulatory effects. Whereas IL-12 and IL-23 are regarded as proinflammatory actors, IL-27, and especially IL-35, manifest anti-inflammatory effects. C. f. Vignali D. A. A. et al. Nat Immunol. 2012; 13 (8): 722-728.

### Interlelukin-12

Interleukin 12 (IL-12) is an interleukin that is naturally produced by dendritic cells, macrophages, neutrophils, and human B-lymphoblastoid cells (NC-37) in response to antigenic stimulation. IL-12 belongs to the family of interleukin-12. The IL-12 family is unique in comprising the only heterodimeric cytokines, which includes IL-12, IL-23, IL-27 and IL-35.^{[2]} Despite sharing many structural features and molecular partners, they mediate surprisingly diverse functional effects.

IL-12 is a heterodimeric cytokine encoded by two separate genes, IL-12A (p35) and IL-12B (p40). The active heterodimer (referred to as ‘p70’), and a homodimer of p40 are formed following protein synthesis. IL-12A is composed of a bundle of four alpha helices. IL-12B has three beta sheet domains.

IL-12 is involved in the differentiation of naive T cells into Th1 cells. It is known as a T cell-stimulating factor, which can stimulate the growth and function of T cells. It stimulates the production of interferon-gamma (IFN-γ) and tumor necrosis factor-alpha (TNF-α) from T cells and natural killer (NK) cells and reduces IL-4 mediated suppression of IFN-γ. T cells that produce IL-12 have a coreceptor, CD30, which is associated with IL-12 activity.

IL-12 plays an important role in the activities of natural killer cells and T lymphocytes. IL-12 mediates enhancement of the cytotoxic activity of NK cells and CD8+ cytotoxic T lymphocytes. There also seems to be a link between IL-2 and the signal transduction of IL-12 in NK cells. IL-2 stimulates the expression of two IL-12 receptors, IL-12R-β1 and IL-12R-β2, maintaining the expression of a critical protein involved in IL-12 signaling in NK cells. Enhanced functional response is demonstrated by IFN-γ production and killing of target cells.

IL-12 also has anti-angiogenic activity, which means it can block the formation of new blood vessels. It does this by increasing production of interferon gamma, which in turn increases the production of a chemokine called inducible protein-10 (IP-10 or CXCL10). IP-10 then mediates this anti-angiogenic effect. Because of its ability to induce immune responses and its anti-angiogenic activity, there has been an interest in testing IL-12 as a possible anti-cancer drug. However, it has not been shown to have substantial activity in the tumors tested to this date. There is a link that may be useful in treatment between IL-12 and the diseases psoriasis & inflammatory bowel disease.

IL-12 binds to the IL-12 receptor, which is a heterodimeric receptor formed by IL-12Rβ1 and IL-12Rβ2. IL-12Rβ2 is considered to play a key role in IL-12 function, since it is found on activated T cells and is stimulated by cytokines that promote Th1 cells development and inhibited by those that promote Th2 cells development. Upon binding, IL-12R-β2 becomes tyrosine phosphorylated and provides binding sites for kinases, Tyk2 and Jak2. These are important in activating critical transcription factor proteins such as STAT4 that are implicated in IL-12 signaling in T cells and NK cells. This pathway is known as the JAK-STAT pathway.

### Interleukin-23

IL-23 is a proinflammatory cytokine of the IL-12 family encoded by the genes IL-23A (p19) and IL12B (p40). Human IL-12B is a 328 amino-acid protein. Human IL-23A is a 189 amino-acid protein. IL-23 is secreted by activated dendritic cells and macrophages and plays a role in cancer, autoimmunity, and pathogen response. It may also enhance angiogenesis.

IL-23 binds to the IL-23 receptor, which is a heterodimeric receptor of IL-23R and IL-12Rβ1. IL23A interacts with IL23R, whereas IL12B interacts with IL12Rβ1. JAK2 and TYK2 are induced upon binding, which promotes the phosphorylation of STAT3 and STAT4.

### Interleukin-27

IL-27 is a heterodimeric cytokine of the IL-12 family with primarily anti-inflammatory effects encoded by IL-27A (p28) gene and Epstein-Barr virus induced gene 3 (EBI3, or IL-27B). Its immunological effects are modulated by its environment and involve a myriad of immune cell types, including B cells, dendritic cells, macrophages, and T cells. IL-27 has shown to manifest an anti-inflammatory effect by stimulating the production of IL-10 via STAT1 and/or STAT3.

IL-27 binds to the IL-27 receptor, which is a heterodimeric receptor of IL27RA (WSX-1) and gp130. 127RA modulates STAT1 activity and promotes the differentiation of CD4⁺ cells as part of the adaptive immune response.

### Interleukin-35

Interleukin 35 (IL-35) is a recently discovered anti-inflammatory cytokine from the IL-12 family. A member of IL-12 family, IL-35 is produced by wide range of regulatory lymphocytes and plays a role in immune suppression. IL-35 can block the development of Th1 and Th17 cells by limiting early T cell proliferation.

IL-35 is a dimeric protein composed of IL-12a and IL-27β chains, which are encoded by two separate genes called IL12A and EBI3 (Epstein-Barr virus-induced gene 3), respectively. The IL-35 receptor consists of IL-12Rβ2 (part of the IL-12R) and gp130 (part of IL-27R) chains. Compared to these two related interleukins, IL-35 is also able to signal through only one of the aforementioned chains. This was proven in vivo when absence of either of the receptor chains did not influence effects of IL-35. On regulatory B-cells, IL-35 signals through the IL-12Rβ2 and IL-27Rα subunits.

EBI3 is a homologue to IL-12 p40 and to the ciliary neurotrophic factor receptor, whose expression is induced in B lymphoblastoid cells by EBV infection.

Secreted by regulatory T-cells (T_{regs}), regulatory B-cells (B_{regs}) or even CD8+ regulatory T cells, IL-35 suppresses inflammatory responses of immune cells. IL-35 is not constitutively expressed in tissues, but the gene encoding IL-35 is transcribed by vascular endothelial cells, smooth muscle cells and monocytes after activation with proinflammatory stimuli. IL-35 has selective activities on different T-cell subsets; it induces proliferation of T_{reg} cell populations but reduces activity of Tₕ17 cell populations.

Studies in mice show the absence of either IL-35 chain from regulatory T_{regs} reduces the cells' ability to suppress inflammation. This has been observed during cell culture experiments and using an experimental model for inflammatory bowel disease. A group of scientists established a CIA (collagen-induced arthritis) mouse model to show suppressive effects of IL-35. Intraperitoneal injection of IL-35 in the tested subjects lowered expression of several factors linked to this disease (such as VEGF and its receptors, TNF-α). The effect of IL-35 in this case seems to be the inhibition of STAT1 signaling pathway. Another experiment performed on a mouse model of EAE has shown, that mice lacking IL-35-producing B cells are unable to recover from the T-cell mediated demyelination but are resistant to infection by the pathogenic intracellular microbe Salmonella typhimurium. In TID (type 1 diabetes), plasma level of IL-35 is lower than healthy individuals. IL-35 production by T_{regs} is decreased in mouse models of TID, and administration of IL-35 prevents the development of experimental TID and reverses established experimental TID. In TID patients with remaining C-peptide, IL-35 production by T_{regs} and B_{regs} is much higher than TID patients with no remaining C-peptide.

It has been shown that IL-35 increases replication of HBV virus both in vitro and in transgenic mice by targeting its transcription factor HNF4α. Given its suppressive function, IL-35 is also involved in tumor progression and tumor immune surveillance. Elevated circulating IL-35 levels have been found in several human tumors such as acute myeloid leukemia, pancreatic ductal adenocarcinoma and colorectal cancer. Moreover, Forkhead box protein 3 (Foxp3) as a transcription factor is an essential molecular marker of regulatory T (T_{reg}) cells. Foxp3 polymorphism (rs3761548) might be involved in cancer progression like gastric cancer through influencing T_{regs} function and the secretion of immunomodulatory cytokines such as IL-10, IL-35, and TGF-β.

The engineered cells may express or be capable of expressing a heterodimeric protein, e.g., a heterodimeric cytokine. In an embodiment, the engineered cell is capable of expressing a cytokine comprising a heterodimer of p35 and p40 (i.e., comprising IL-12) or a functional fragment thereof. In an embodiment, the engineered cell is capable of expressing a cytokine comprising a heterodimer of p19 and p40 (i.e., comprising IL-23) or a functional fragment thereof. In an embodiment, the engineered cell is capable of expressing a cytokine comprising a heterodimer of p28 and EBI3 (i.e., comprising IL-27) or a functional fragment thereof. In an embodiment, the engineered cell is capable of expressing a cytokine comprising a heterodimer of p35 and EBI3 (i.e., comprising IL-35) or a functional fragment thereof. In an embodiment, the engineered cell is capable of expressing IL-12 or a functional fragment thereof. In an embodiment, the engineered cell is capable of expressing IL-23 or a functional fragment thereof. In an embodiment, the engineered cell is capable of expressing IL-27 or a functional fragment thereof. In an embodiment, the engineered cell is capable of expressing IL-35 or a functional fragment thereof.

In an embodiment, the engineered cell is capable of expressing a heterodimer comprising p35 or a functional fragment thereof. In an embodiment, the engineered cell is capable of expressing a heterodimer comprising p40 or a functional fragment thereof. In an embodiment, the engineered cell is capable of expressing a heterodimer comprising p28 or a functional fragment thereof. In an embodiment, the engineered cell is capable of expressing a heterodimer comprising EBI3 or a functional fragment thereof.

In an embodiment, the engineered cell is capable of expressing a protein comprising p35 or functional fragment thereof. In an embodiment, the engineered cell is capable of expressing a protein comprising p40 or a functional fragment thereof. In an embodiment, the engineered cell is capable of expressing a protein comprising p28 or a functional fragment thereof. In an embodiment, the engineered cell is capable of expressing a protein comprising EBI3 or a functional fragment thereof.

In an embodiment, the engineered cell is capable of expressing a cytokine or cytokine subunit with a least 60%, at least 65%, at least 70%, at least 75%, at least 80%, at least 85%, at least 90%, at least 91%, at least 92%, at least 93%, at least 94%, at least 95%, at least 96%, least 97%, at least 98%, or at least 99% identity to a cytokine or cytokine subunit described herein, or a functional fragment thereof, e.g., at least 80%, at least 85%, at least 90%, at least 91%, at least 92%, at least 93%, at least 94%, at least 95%, at least 96%, at least 97%, at least 98%, or at least 99% identity to a cytokine or cytokine subunit described herein, or a functional fragment thereof.

In an embodiment, the cytokine or cytokine subunit comprises an amino acid sequence as described in Table 1. In an embodiment, the cytokine or cytokine subunit comprises an amino acid sequence with at least 60%, at least 65%, at least 70%, at least 75%, at least 80%, at least 85%, at least 90%, at least 91%, at least 92%, at least 93%, at least 94%, at least 95%, at least 95%, at least 96%, at least 97%, at least 98%, or at least 99% of the amino acid sequence as described in Table 1. In an embodiment, the cytokine or cytokine subunit comprises an amino acid sequence of at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% of the amino acid sequence according to SEQ ID NO: 1. In an embodiment, the cytokine or cytokine subunit comprises an amino acid sequence of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the amino acid sequence according to SEQ ID NO: 2. In an embodiment, the cytokine or cytokine subunit comprises an amino acid sequence of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the amino acid sequence according to SEQ ID NO: 3. In an embodiment, the cytokine or cytokine subunit comprises an amino acid sequence of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the amino acid sequence according to SEQ ID NO: 4. In an embodiment, the cytokine or cytokine subunit comprises an amino acid sequence of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the amino acid sequence according to SEQ ID NO: 5.

In an embodiment, the cytokine or cytokine subunit comprises an amino acid sequence with 1, 2, 3, 4, 5, 6, 7, 8, 9, 10 or more mutations relative to an amino acid sequence of a cytokine or a cytokine subunit as described herein, or a functional fragment thereof. In an embodiment, the cytokine or cytokine subunit comprises an amino acid sequence with 1, 2, 3, 4, 5, 6, 7, 8, 9, 10 or more mutations relative to an amino acid sequence of a cytokine or a cytokine subunit as described in Table 1. In an embodiment, the cytokine or cytokine subunit comprises an amino acid sequence with 1, 2, 3, 4, 5. 6, 7, 8, 9, 10 or more mutations relative to an amino acid sequence of SEQ ID NO: 1. In an embodiment, the cytokine or cytokine subunit comprises an amino acid sequence with 1, 2, 3, 4, 5. 6, 7, 8, 9, 10 or more mutations relative to an amino acid sequence of SEQ ID NO: 2. In an embodiment, the cytokine or cytokine subunit comprises an amino acid sequence with 1, 2, 3, 4, 5. 6, 7, 8, 9, 10 or more mutations relative to an amino acid sequence of SEQ ID NO: 3. In an embodiment, the cytokine or cytokine subunit comprises an amino acid sequence with 1, 2, 3, 4, 5. 6, 7, 8, 9, 10 or more mutations relative to an amino acid sequence of SEQ ID NO: 4. In an embodiment, the cytokine or cytokine subunit comprises an amino acid sequence with 1, 2, 3, 4, 5. 6, 7, 8, 9, 10 or more mutations relative to an amino acid sequence of SEQ ID NO: 5.

In an embodiment, the mRNA transcript encoding for the protein comprising the cytokine or cytokine subunit comprises an internal ribosome entry site (IRES) or a functional fragment thereof. In an embodiment, a CAG promoter is adjacent to a DNA sequencing encoding for the protein comprising the cytokine or functional variant thereof. In an embodiment, the amino acid sequence of the protein comprising the cytokine or cytokine subunit comprises a linker peptide, e.g., a linker peptide comprising the sequence IEGRMD or (GGGS)₃. In an embodiment, the amino acid sequence of the protein comprising the cytokine or cytokine subunit comprises a fragment crystallizable region (Fc region) of an antibody, e.g., mIgG21, mIgG2a, or mIgG21. In an embodiment, the amino acid sequence of the protein comprising cytokine or cytokine subunit comprises a self-cleaving peptide, e.g., P2A, or a functional fragment thereof.

In an embodiment, the engineered cell is capable of expressing an IL12p35 expression plasmid encoding for an IL12p35 fusion protein comprising p35, IEGRMD linker, and mIgG2a or a functional fragment thereof. In an embodiment, the engineered cell is capable of expressing an IL-23 expression plasmid encoding for an IL-23 fusion protein comprising p19, and p40, or a functional fragment thereof. In an embodiment, the engineered cell is capable of expressing an IL-27 plasmid encoding for an IL-27 fusion protein comprising p28, and EBI3 or a functional fragment thereof. In an embodiment, the engineered cell is capable of expressing a flexi-IL-35 Fc expression plasmid encoding for an IL-35 fusion protein comprising EBI3, (GGGS) 3 flexible linker, p35, IEGRMD linker, and mIgG2a or a functional fragment thereof. In an embodiment, the engineered cell is capable of expressing a flexi-IL-35 expression plasmid encoding for an IL-35 fusion protein comprising EBI3, (GGGS)₃ flexible linker, and p35 or a functional fragment thereof. In an embodiment, the engineered cell is capable of expressing an IL-35 Fc expression plasmid encoding an IL-35 fusion protein comprising p35, IEGRMD linker, mIgG2a, EBI3, IEGMRD linker, and mIgG21 or a functional fragment thereof. In an embodiment, the engineered cell is capable of expressing a reverse IL-35 plasmid encoding for an IL-35 fusion protein comprising EBI3 and p35 or a functional fragment thereof.

TABLE 1
Exemplary amino acid sequences of human interleukin-12 family subunits

| IL-12 Family | |
|---|---|
| Protein | |
| Subunit | Primary Amino Acid Sequence |
| SEQ ID NO: 1 | MLGSRAVMLLLLLPWTAQGRAVPGGSSPAWTQCQQLSQKLCTL |
| p19 | AWSAHPLVGHMDLREEGDEETTNDVPHIQCGDGCDPQGLRDNSQ |
| | FCLQRIHQGLIFYEKLLGSDIFTGEPSLLPDSPVGQLHASLLGLSQL |
| | LQPEGHHWETQQIPSLSPSQPWQRLLLRFKILRSLQAFVAVAARVF |
| | AHGAATLSP |
| SEQ ID NO: 2 | MGQTAGDLGWRLSLLLLPLLLVQAGVWGFPRPPGRPQLSLQELR |
| p28 | REFTVSLHLARKLLSEVRGQAHRFAESHLPGVNLYLLPLGEQLPD |
| | VSLTFQAWRRLSDPERLCFISTTLQPFHALLGGLGTQGRWTNMER |
| | MQLWAMRLDLRDLQRHLRFQVLAAGFNLPEEEEEEEEEEEEERK |
| | GLLPGALGSALQGPAQVSWPQLLSTYRLLHSLELVLSRAVRELLL |
| | LSKAGHSVWPLGFPTLSPQP |
| SEQ ID NO: 3 | MCPARSLLLVATLVLLDHLSLARNLPVATPDPGMFPCLHHSQNLL |
| p35 | RAVSNMLQKARQTLEFYPCTSEEIDHEDITKDKTSTVEACLPLELT |
| | KNESCLNSRETSFITNGSCLASRKTSFMMALCLSSIYEDLKMYQVE |
| | FKTMNAKLLMDPKRQIFLDQNMLAVIDELMQALNFNSETVPQKS |
| | SLEEPDFYKTKIKLCILLHAFRIRAVTIDRVMSYLNAS |
| SEQ ID NO: 4 | MCHQQLVISWFSLVFLASPLVAIWELKKDVYVVELDWYPDAPGE |
| p40 | MVVLTCDTPEEDGITWTLDQSSEVLGSGKTLTIQVKEFGDAGQYT |
| | CHKGGEVLSHSLLLLHKKEDGIWSTDILKDQKEPKNKTFLRCEAK |
| | NYSGRFTCWWLTTISTDLTFSVKSSRGSSDPQGVTCGAATLSAER |
| | VRGDNKEYEYSVECQEDSACPAAEESLPIEVMVDAVHKLKYENY |
| | TSSFFIRDIIKPDPPKNLQLKPLKNSRQVEVSWEYPDTWSTPHSYFS |
| | LTFCVQVQGKSKREKKDRVFTDKTSATVICRKNASISVRAQDRYY |
| | SSSWSEWASVPCS |
| SEQ ID NO: 5 | MTPQLLLALVLWASCPPCSGRKGPPAALTLPRVQCRASRYPIAVD |
| EBI3 | CSWTLPPAPNSTSPVSFIATYRLGMAARGHSWPCLQQTPTSTSCTI |
| | TDVQLFSMAPYVLNVTAVHPWGSSSSFVPFITEHIIKPDPPEGVRLS |
| | PLAERQLQVQWEPPGSWPFPEIFSLKYWIRYKRQGAARFHRVGPI |
| | EATSFILRAVRPRARYYVQVAAQDLTDYGELSDWSLPATATMSL |
| | GK |

The engineered cell is capable of secreting a protein, e.g., a cytokine. In an embodiment, the cytokine is a cytokine of the IL-12 family, e.g., IL-12, IL-23, IL-27, or IL-35. In an embodiment, the cytokine is IL-12. In an embodiment, the cytokine is IL-23. In an embodiment, the cytokine is IL-27. In an embodiment, the cytokine is IL-35.

Functionals variants of a polypeptide (e.g., a protein) may comprise one or more mutations, wherein the functional variant maintains at least some activity of the native polypeptide, e.g., a protein subunit of SEQ ID NOS: 1-5 in Table 1. In an embodiment, the functional variant maintains at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the activity of the corresponding polypeptide as described herein (e.g., the protein subunits of SEQ ID NOS: 1-5 in Table 1.) In an embodiment, the functional variant has at least 100%, at least 150%, at least 200%, at least 300%, at least 400%, at least 500%, or at least 1000% of the activity of the corresponding polypeptide as described herein (e.g., the protein subunits of SEQ ID NOS: 1-5 in Table 1). Polypeptide activity may be evaluated using conventional assays known to a person of skill in the art.

Functional variants of a polypeptide may comprise mutations that include additions, deletions, and substitutions of one or more amino acid residues. Mutations may be introduced into a native amino acid sequence by conventional techniques known to individuals of skill in the art, e.g., PCR-directed mutagenesis and site-directed mutagenesis. Other mutagenesis techniques include clustered regularly-interspaced short palindromic repeats (CRISPR)/Cas platforms. Cf. Wiedenheft et al. Nature. 2012, 482:331-8.

Functional variants of a polypeptide may include conserved mutations, wherein one or more amino acid residues of the native polypeptide is substituted with an amino acid comprising a side chain with similar chemistry. For example, amino acids containing acidic side chains include aspartic acid (aspartate) and glutamic acid (glutamate). Amino acids containing basic side chains include arginine, histidine, and lysine. Amino acids containing nonpolar side chains include alanine, glycine, isoleucine, leucine, methionine, proline, phenylalanine, tryptophan, and valine. Amino acids containing polar side chains include asparagine, cysteine, glutamine, serine, threonine, or tyrosine.

Functional variants may have altered enzymatic activity or characteristics relative to the native polypeptide (e.g., a protein subunit of SEQ ID NOS: 1-5 in Table). For example, the functional variant may have altered affinity for a secondary messenger protein, e.g., a Janus kinase (JAK), e.g., JAK1, JAK2, JAK3, and TYK2, or a signal transducer activator of transcription (STAT) protein, e.g., STAT1, STAT2, STAT3, STAT4, STAT5, STAT5A, STAT5B, and STAT6.

Functional variants may have different expression levels or stability relative to the native polypeptide (e.g., a protein subunit of SEQ ID NOS: 1-5 in Table 1). For example, the functional variant may have an increased stability at a certain temperature or pH relative to the native polypeptide. Alternatively, the functional variant may have an increased expression level in a certain host cell, e.g., RPE cells, relative to the native polypeptide. For example, trafficking of the functional variant from the endoplasmic reticulum or Golgi apparatus to another intracellular compartment, e.g., the cellular membrane, may be increased or diminished relative to the native polypeptide.

The engineered cell is also contemplated to secrete a cytokine that is not a member of the IL-12 family. Exemplary proinflammatory cytokines include interferon gamma (IFNγ), interleukin-1 alpha (IL-1α), interleukin-1 beta (IL-1β), interleukin-2 (IL-2), interleukin-1 beta (IL-1β), interleukin-6 (IL-6), interleukin-8 (IL-8), interleukin-12 (IL-12), interleukin-17A (IL-17A), interleukin 18 (IL-18), interleukin-23 (IL-23) interleukin-33 (IL-33), interleukin-36 alpha (IL-36α), interleukin-36 beta (IL-36β), interleukin-36 gamma (IL-36γ), tumor necrosis factor alpha (TNFα), and tumor necrosis factor alpha (TNFα).

Exemplary anti-inflammatory cytokines include interferon beta (IFNβ), interleukin-2 (IL-2), interleukin 4 (IL-4), interleukikn-5 (IL-5) interleukin-6 (IL-6), interleukin-10 (IL-10), interleukin-13 (IL-13), interleukin-19 (IL-19), interleukin-27 (IL-27), interleukin-33 (IL-33), interleukin-35 (IL-35), interleukin-37 (IL-36), interleukin-38 (IL-38), and transforming growth factor beta (TGFβ).

Exemplary immunomodulatory cytokines include interferon alpha (IFNα), interleukin-1 receptor antagonist protein (IL-1Ra), interleukin-27 (IL-27), and interleukin-36 receptor antagonist (IL-36Ra).

The engineered cell is further contemplated in some embodiments to secrete a chemokine. Exemplary proinflammatory chemokines include chemokine (C—C motif) ligand 2 (CCL2), chemokine (C—C motif) ligand 3, (CCL3), chemokine (C—C motif) ligand 5 (CCL5), chemokine (C—C motif) ligand 20 (CCL20), chemokine (C—X—C motif) ligand 1 (CXCL1), chemokine (C—X—C motif) ligand 4 (CXCL4), chemokine (C—X—C motif) ligand 5 (CXCL5), chemokine (C—X—C motif) ligand 6 (CXCL6), chemokine (C—X—C motif) ligand 7 (CXCL7), chemokine (C—X—C motif) ligand 8 (CXCL8), chemokine (C—X—C motif) ligand 9 (CXCL9), and chemokine (C—X—C motif) ligand 10 (CXCL10).

Exemplary anti-inflammatory chemokines include (C—C motif) ligand 17 (CCL17), chemokine (C—C motif) ligand 18 (CCL18), chemokine (C—C motif) ligand 19 (CCL19), chemokine (C—C motif) ligand 21 (CCL21), chemokine (C—X—C motif) ligand 12 (CXCL12), chemokine (C—X—C motif) ligand 13 (CXCL13), and chemokine (C—X—C motif) ligand 16 (CXCL16).

### Implantable Devices

An implantable construct described herein comprises a material that reduces or inhibits a reaction (e.g., such as an immunomodulatory reaction) with or on an antigenic or therapeutic agent disposed within. In an embodiment, the antigenic or therapeutic agent is released from the device. For example, an implantable construct comprises a zone or layer that shields an antigenic agent from exposure to the surrounding milieu, such as host tissue, host cells, or host cell products. In an embodiment, an implantable construct minimizes the effect of a host response (e.g., an immune response) directed at an antigenic or therapeutic agent disposed within, e.g., as compared with a similar antigenic agent that is not disposed within an implantable construct.

The implantable construct make take any shape. The surface may be a flat surface or a curved surface and can take a variety of more complex forms such a sphere, a tube (e.g., inside or outside of the tube), a bead, a rod, a wire, or even more complex 3-D structures such as medical devices.

The implantable construct may comprise a permeable, semi-permeable, or impermeable material to control the flow of solution in and out of the implantable construct. The implantable construct may comprise a permeable, semi-permeable, or impermeable material to adopt to the shape or size of its surroundings. For example, the material may be permeable or semi-permeable to allow free passage of small molecules, such as nutrients and waste products, in and out of the construct. In addition, the material may be permeable or semi-permeable to allow the transport of an antigenic or therapeutic agent, out of the implantable construct. In addition, the material may be permeable or semi-permeable to allow the transport of an cytokine or other protein therapeutic, out of the implantable construct. Exemplary materials include polymers, metals, ceramics, and combinations thereof.

In an embodiment, the implantable construct comprises a polymer (e.g., a naturally occurring polymer or a synthetic polymer). For example, a polymer may comprise polystyrene, polyester, polycarbonate, polyethylene, polypropylene, polyfluorocarbon, nylon, polyacetylene, polyvinyl chloride (PVC), polyolefin, polyurethane, polyacrylate, polymethacrylate, polyacrylamide, polymethacrylamide, polymethyl methacrylate, poly(2-hydroxyethyl methacrylate), polysiloxane, polydimethylsiloxane (PDMS), polyhydroxyalkanoate, PEEK®, polytetrafluoroethylene, polyethylene glycol, polysulfone, polyacrylonitrile, collagen, cellulose, cellulosic polymers, polysaccharides, polyglycolic acid, poly(L-lactic acid) (PLLA), poly(lactic glycolic acid) (PLGA), polydioxanone (PDA), poly(lactic acid), hyaluronic acid, agarose, alginate, chito san, or a blend or copolymer thereof. In an embodiment, the implantable construct comprises a polysaccharide (e.g., alginate, cellulose, hyaluronic acid, or chitosan).

In an embodiment, the implantable construct comprises a polysaccharide, e.g., hyaluronic acid or alginate. In an embodiment, the implantable construct comprises alginate. Alginate is a naturally occurring polymer comprising β-(1-4)-linked mannuronic acid and guluronic acid residues, and as a result of its high density of negatively charged carboxylates, may be cross-linked with certain cations to form a larger structure, such as a hydrogel. In some embodiments, the average molecular weight of the polymer is from about 2 kDa to about 500 kDa (e.g., from about 2.5 kDa to about 175 kDa, from about 5 kDa about 150 kDa, from about 10 kDa to about 125 kDa, from about 12.5 kDa to about 100 kDa, from about 15 kDa to about 90 kDa, from about 17.5 kDa to about about 80 kDa, from about 20 kDa to about 70 kDa, from about 22.5 kDa to about 60 kDa, or from about 25 kDa to about 50 kDa). The implantable construct may comprise at least 0.5%, 1%, 2%, 3%, 4%, 5%, 7.5%, 10%, 15%, 20%, 30%, 40%, 50%, 60%, 70%, 80% or more of a polymer, e.g., a polymer described herein. In an embodiment, the polymer is an alginate polymer. In an embodiment, the alginate is an ultrapure alginate (e.g., SLG20 alginate).

In an embodiment, the implantable construct comprises a metal or a metallic alloy. Exemplary metals or metallic alloys include titanium (e.g., nitinol, nickel titanium alloys, thermo-memory alloy materials), platinum, platinum group alloys, stainless steel, tantalum, palladium, zirconium, niobium, molybdenum, nickel-chrome, cobalt, tantalum, chromium molybdenum alloys, nickel-titanium alloys, and cobalt chromium alloys. In an embodiment, the implantable construct comprises stainless steel grade. The implantable construct may comprise at least 0.5%, 1%, 2%, 3%, 4%, 5%, 7.5%, 10%, 15%, 20%, 30%, 40%, 50%, 60%, 70%, 80% or more of a metal or metallic alloy, e.g., a metal or metallic alloy described herein.

In an embodiment, the implantable construct comprises a ceramic. Exemplary ceramics include carbide, nitride, silica, or oxide materials (e.g., titanium oxides, hafnium oxides, iridium oxides, chromium oxides, aluminum oxides, and zirconium oxides). The implantable construct may comprise at least 0.5%, 1%, 2%, 3%, 4%, 5%, 7.5%, 10%, 15%, 20%, 30%, 40%, 50%, 60%, 70%, 80% or more of a ceramic, e.g., a ceramic described herein.

In an embodiment, the implantable construct may comprise glass. The implantable construct may comprise at least 0.5%, 1%, 2%, 3%, 4%, 5%, 7.5%, 10%, 15%, 20%, 30%, 40%, 50%, 60%, 70%, 80% or more glass.

A material within an implantable construct may be further modified, for example, with a chemical modification. For example, a material may be coated or derivatized with a chemical modification that provides a specific feature, such as an immunomodulatory or antifibrotic feature. Exemplary chemical modifications include small molecules, peptides, proteins, nucleic acids, lipids, or oligosaccharides. The implantable construct may comprise at least 0.5%, 1%, 2%, 3%, 4%, 5%, 7.5%, 10%, 15%, 20%, 30%, 40%, 50%, 60%, 70%, 80% or more of a material that is chemically modified, e.g., with a chemical modification described herein.

In some embodiments, the material is chemically modified with a specific density of modifications. The specific density of chemical modifications may be described as the average number of attached chemical modifications per given area. For example, the density of a chemical modification on a material in, on, or within an implantable construct described herein may be 0.01, 0.1, 0.5, 1, 5, 10, 15, 20, 50, 75, 100, 200, 400, 500, 750, 1,000, 2,500, or 5,000 chemical modifications per square μm or square mm.

In an embodiment, the chemical modification of a material may include a linker or other attachment moiety. These linkers may include a cross-linker, an amine-containing linker, an ester-containing linker, a photolabile linker, a peptide-containing linker, a disulfide-containing linker, an amide-containing linker, a phosphoryl-containing linker, or a combination thereof. A linker may be labile (e.g., hydrolysable). Exemplary linkers or other attachment moieties is summarized in Bioconjugate Techniques (3^{rd} edition, Greg T. Hermanson, Waltham, MA: Elsevier, Inc, 2013), which is incorporated herein by reference in its entirety.

In some embodiments, implantable constructs comprising a polymer and a plurality of cells have been engineered to deliver a therapeutic agent, e.g., a protein, e.g., a cytokine. In some embodiments, the polymer forms a hydrogel, or is capable of forming a hydrogel. In some embodiments, the polymer forms a hydrogel, or is capable of forming a hydrogel, that encapsulates a plurality of engineered cells.

A hydrogel is a crosslinked structure of polymers comprising a substantial amount of water, with a myriad of applications in drug delivery, biomedical devices, sensors, tissue engineering, and semiconductors due to its unique combination of physical and mechanical properties as varied as biodegradability, biocompatibility, stimuli-responsiveness (e.g., thermo- or pH responsiveness), non-Newtonian behavior, viscoelasticity, and its ability to self-heal. Hydrogels may comprise a natural polymer or a synthetic polymer. In some embodiments, the hydrogel comprises a natural polymer, wherein the natural polymer is a polypeptide. Exemplary polymers and copolymers capable of forming a hydrogel include, without limitation, alginate, alginate-polyethylene glycol (alginate-PEG), alginate-polyacrylamide (alginate-PAAm), α-cyclodextrin, α-cyclodextrin-polyethylene glycol (α-cyclodextrin-PEG), chitosan, collagen, fibrin, heparin, hyaluronic acid (HA), polyethylene glycol (PEG), polyacrylic acid (PAA), polyacrylamide (PAAm), polyacrylamide-ferrocene (PAAm-Fc), poly(L-lysine)/polyacrylic acid (PLL/PAA), poly(N-isopropylacylamide) (PNIPAAM), poly((N-isopropylacrylamide)-co-(sodium acrylic acid)) (P(NIPAAM-co-AAcNa)), polyvinyl alcohol-polyacrylamide (PVA-PAAm), and poly(vinyl pyrrolidine) (PVP). In some embodiments, the hydrogel polymer comprises any of the hydrogel-forming polymers disclosed in Zhang, Y. S. Science. 2017, 356, 6337, eaaf3627.

Implantable constructs may comprise a polymer which is biocompatible. In some embodiments, the polymer is biocompatible and hydrogel-forming, or capable of forming a hydrogel. In some embodiments, the biocompatible polymer is a naturally derived polymer. In some embodiments, the biocompatible polymer is a synthetic polymer.

The biocompatible polymer may be a natural polymer. Exemplary natural biocompatible polymers include alginic acid and pharmaceutically acceptable salts thereof (e.g., barium alginate calcium alginate, sodium alginate, potassium alginate and the like), cellulose, cellulose ethers (e.g., cellulose hydrocolloids), chitosan, chondroitin sulphate, starch, collagen, hyaluronic acid (HA), inorganic polyphosphosphate, and poly(hydroxybutyrate) (PHB).

The biocompatible polymer may be a synthetic polymer. Exemplary synthetic biodegradable polymers include polycaprolactone (PCL), poly(hydroxybutyrate-co-hydroxyvalerate), poly(γ-glutamic acid), polyglycolic acid (PGA), polylactic acid (PLA), poly(L-lactic acid) (PLLA), and poly(lactic-co-glycolic acid) (PLGA).

The biocompatible polymer may be a poly(amino acid). Exemplary biocompatible amino acids include polylysine, e.g., poly(D/L-lysine), poly(L-lysine), and poly(D-lysine).

The biocompatible polymer may comprise a polyethylene glycol copolymer. Exemplary biocompatible polyethylene glycol copolymers include poly(ethylene glycol)-b-hyaluronic acid (PEG-HA), poly(ethylene glycol)-b-polycaprolactone (PEG-PCL), poly(ethylene glycol)-b-poly(glutamic acid) (PEG-PGA), poly(ethylene glycol)-b-polylactic acid (PEG-PLA), poly(ethylene glycol)-b-poly(glycolic-co-lactic acid) (PEG-PLGA), poly(ethylene glycol)-b-poly(L-lactic acid) (PEG-PLLA), and polyethylene glycol-b-poly(ethyleneimine) (PEG-PEI).

The biocompatible polymer may be a polyanhydride. Exemplary biocompatible polyanhydrides include poly(adipic anhydride), poly(azelaic anhydride), poly(dodecanedioc anhydride), poly(dodecane carboxylic acid), poly(fumaric anhydride), poly(fumaric anhydride-sebacic anhydride), poly(furmaric anhydride dodecanedioc anhydride) poly(fumaric anhydride adipic anhydride), poly(hexadecenoic anhydride), poly(isophthalic anhydride), poly(pimelic anhydride), poly(sebacic anhydride), poly(sebacic acid-co-1,3-bis(p-carboxyphenoxy) propane (P(CPP-SA)), poly(suberic anhydride), and poly(terephthalic anhydride) or a copolymer thereof. In some embodiments, the biodegradable polyanhydride polymer comprises any of the polyanhydrides disclosed in Reddy, B. G. Biomacromolecules. 2022, 23, 12, 4959-4984.

The biocompatible polymer may be a poly(diol citrate), e.g., a poly(diol citrate) elastomer as disclosed in U.S. Pat. No. 8,911,720, which is herein incorporated by reference in its entirety.

The biocompatible polymer may be a polyphosphazene or a derivative thereof, e.g., a polyphosphazene disclosed in U.S. Pat. Nos. 11,584,828; 5,562,909; 5,562,099; and 5,500,161, which are herein incorporated by reference in their entirety.

The biocompatible polymer may be a polyurethane, e.g., a biocompatible polyurethane or derivative thereof as described in U.S. Pat. No. 9,540,478, which is herein incorporated by reference in its entirety.

The biocompatible polymer may be a polycarbonate, e.g., a biodegradable polycarbonate, copolymer, or derivative thereof, as disclosed in U.S. Pat. No. 9,901,649, which is herein incorporated by reference in its entirety.

The biocompatible polymer may be a polyorthoester, e.g., a biodegradable polyorthoester or derivative thereof, as disclosed in U.S. Pat. No. 11,413,350, which is herein incorporated by reference in its entirety.

In an embodiment, the biocompatible polymer comprises any of the biocompatible polymers disclosed in Doppalapudi S. et al. Polym. Adv. Technol. 2014, 25, 5, 427-435.

Fabrication of the implantable constructs allows for diameters between about 5 μm to about 3000 μm for delivery of a protein such as a cytokine. In an embodiment, the implantable construct diameter is between about 5 μm to about 3000 μm, about 6 μm to about 3000 μm, about 7 μm to about 3000 μm, about 8 μm to about 3000 μm, about 9 μm to about 3000 μm, about 10 μm to about 3000 μm, about 20 μm to about 3000 μm, about 30 μm to about 3000 μm, about 40 μm to about 3000 μm, about 50 μm to about 3000 μm, about 100 μm to about 3000 μm, about 200 μm to about 3000 μm, about 300 μm to about 3000 μm, about 400 μm to about 3000 μm, about 500 μm to about 3000 μm, about 1000 μm to about 3000 μm, or about 2000 μm to about 3000 μm. In an embodiment, the implantable construct diameter is between about 6 μm to about 3000 μm. In an embodiment, the implantable construct diameter is between about 7 μm to about 3000 μm. In an embodiment, the implantable construct diameter is between about 8 μm to about 3000 μm. In an embodiment, the implantable construct diameter is between about 9 μm to about 3000 μm. In an embodiment, the implantable construct diameter is between about 10 μm to about 3000 μm. In an embodiment, the implantable construct diameter is between about 20 μm to about 3000 μm. In an embodiment, the implantable construct diameter is between about 30 μm to about 3000 μm. In an embodiment, the implantable construct diameter is between about 40 μm to about 3000 μm. In an embodiment, the implantable construct diameter is between about 50 μm to about 3000 μm. In an embodiment, the implantable construct diameter is between about 100 μm to about 3000 μm. In an embodiment, the implantable construct diameter is between about 200 μm to about 3000 μm. In an embodiment, the implantable construct diameter is between about 300 μm to about 3000 μm. In an embodiment, the implantable construct diameter is between about 400 μm to about 3000 μm. In an embodiment, the implantable construct diameter is between about 500 μm to about 3000 μm. In an embodiment, the implantable construct diameter is between about 1000 μm to about 3000 μm. In an embodiment, the implantable construct diameter is between about 2000 μm to about 3000 μm.

In an embodiment, the implantable construct diameter is about 150 μm, about 300 μm, about 600 μm, about 800 μm, about 1200 μm, or about 1700 μm. In an embodiment, the implantable construct diameter is about 150 μm. In some embodiments, the implantable construct diameter is about 300 μm. In an embodiment, the implantable construct diameter is about 600 μm. In an embodiment, the implantable construct diameter is about 800 μm. In an embodiment, the implantable construct diameter is about 1200 μm. In some embodiments, the implantable construct diameter is about 1700 μm.

In an embodiment, the implantable construct diameter is greater than about 100 μm. In an embodiment, the implantable construct diameter is greater than about 150 μm, about 200 μm, about 300 μm, about 400 μm, about 500 μm, about 600 μm, about 700 μm, about 800 μm, about 900 μm, about 1000 μm, about 1100 μm, about 1200 μm, about 1300 μm, about 1400 μm, about 1500 μm, about 1600 μm, about 1700 μm, about 1800 μm, about 1900 μm, or about 2000 μm. In an embodiment, the implantable construct diameter is greater than about 150 μm. In an embodiment, the implantable construct diameter is greater than about 200 μm. In an embodiment, the implantable construct diameter is greater than about 300 μm. In an embodiment, the implantable construct diameter is greater than about 400 μm. In an embodiment, the implantable construct diameter is greater than about 500 μm. In an embodiment, the implantable construct diameter is greater than about 600 μm. In an embodiment, the implantable construct diameter is greater than about 700 μm. In an embodiment, the implantable construct diameter is greater than about 800 μm. In an embodiment, the implantable construct diameter is greater than about 900 μm. In an embodiment, the implantable construct diameter is greater than about 1000 μm. In an embodiment, the implantable construct diameter is greater than about 1100 μm. In an embodiment, the implantable construct diameter is greater than about 1200 μm. In an embodiment, the implantable construct diameter is greater than about 1300 μm. In an embodiment, the implantable construct diameter is greater than about 1400 μm. In an embodiment, the implantable construct diameter is greater than about 1500 μm. In an embodiment, the implantable construct diameter is greater than about 1600 μm. In an embodiment, the implantable construct diameter is greater than about 1700 μm. In an embodiment, the implantable construct diameter is greater than about 1800 μm. In an embodiment, the implantable construct diameter is greater than about 1900 μm. In an embodiment, the implantable construct diameter is greater than about 2000 μm.

In an embodiment, the implantable construct diameter is between about 100 μm and 2000 μm. In an embodiment, the implantable construct diameter is between about 100 μm and about 1900 μm, about 100 μm and about 1800 μm, about 100 μm and about 1700 μm, about 100 μm and about 1600 μm, about 100 μm and about 1500 μm, about 100 μm and about 1400 μm, about 100 μm and about 1300 μm, about 100 μm and about 1200 μm, about 100 μm and about 1100 μm, about 100 μm and about 1000 μm, about 100 μm and about 900 μm, about 100 μm and about 800 μm, about 100 μm and about 700 μm, about 100 μm and about 600 μm, about 100 μm and about 500 μm, about 100 μm and about 400 μm, about 100 μm and about 300 μm, or about 100 μm and about 200 μm. In an embodiment, the implantable construct diameter is between about 100 μm and about 1900 μm. In an embodiment, the implantable construct diameter is between about 100 μm and about 1800 μm. In an embodiment, the implantable construct diameter is between about 100 μm and about 1700 μm. In an embodiment, the implantable construct diameter is between about 100 μm and about 1600 μm. In an embodiment, the implantable construct diameter is between about 100 μm and about 1500 μm. In an embodiment, the implantable construct diameter is between about 100 μm and about 1400 μm. In an embodiment, the implantable construct diameter is between about 100 μm and about 1300 μm. In an embodiment, the implantable construct diameter is between about 100 μm and about 1200 μm. In an embodiment, the implantable construct diameter is between about 100 μm and about 1100 μm. In an embodiment, the implantable construct diameter is between about 100 μm and about 1000 μm. In an embodiment, the implantable construct diameter is between about 100 μm and about 900 μm. In an embodiment, the implantable construct diameter is between about 100 μm and about 800 μm. In an embodiment, the implantable construct diameter is between about 100 μm and about 700 μm. In an embodiment, the implantable construct diameter is between about 100 μm and about 600 μm. In an embodiment, the implantable construct diameter is between about 100 μm and about 500 μm. In an embodiment, the implantable construct diameter is between about 100 μm and about 400 μm. In an embodiment, the implantable construct diameter is between about 100 μm and about 300 μm. In an embodiment, the implantable construct diameter is between about 100 μm and about 200 μm.

In an embodiment, the implantable construct diameter is about 150 μm.

In an embodiment, the implantable construct diameter is between about 200 μm and 2000 μm. In an embodiment, the implantable construct diameter is between about 200 μm and about 1900 μm, about 200 μm and about 1800 μm, about 200 μm and about 1700 μm, about 200 μm and about 1600 μm, about 200 μm and about 1500 μm, about 200 μm and about 1400 μm, about 200 μm and about 1300 μm, about 200 μm and about 1200 μm, about 200 μm and about 1100 μm, about 200 μm and about 1000 μm, about 200 μm and about 900 μm, about 200 μm and about 800 μm, about 200 μm and about 700 μm, about 200 μm and about 600 μm, about 200 μm and about 500 μm, about 200 μm and about 400 μm, or about 200 μm and about 300 μm. In an embodiment, the implantable construct diameter is between about 200 μm and about 1900 μm. In an embodiment, the implantable construct diameter is between about 200 μm and about 1800 μm. In an embodiment, the implantable construct diameter is between about 200 μm and about 1700 μm. In an embodiment, the implantable construct diameter is between about 200 μm and about 1600 μm. In an embodiment, the implantable construct diameter is between about 200 μm and about 1500 μm. In an embodiment, the implantable construct diameter is between about 200 μm and about 1400 μm. In an embodiment, the implantable construct diameter is between about 200 μm and about 1300 μm. In an embodiment, the implantable construct diameter is between about 200 μm and about 1200 μm. In an embodiment, the implantable construct diameter is between about 200 μm and about 1100 μm. In an embodiment, the implantable construct diameter is between about 200 μm and about 1000 μm. In an embodiment, the implantable construct diameter is between about 200 μm and about 900 μm. In an embodiment, the implantable construct diameter is between about 200 μm and about 800 μm. In an embodiment, the implantable construct diameter is between about 200 μm and about 700 μm. In an embodiment, the implantable construct diameter is between about 200 μm and about 600 μm. In an embodiment, the implantable construct diameter is between about 200 μm and about 500 μm. In an embodiment, the implantable construct diameter is between about 200 μm and about 400 μm. In an embodiment, the implantable construct diameter is between about 200 μm and about 300 μm.

In an embodiment, the implantable construct diameter is about 300 μm.

In an embodiment, the implantable construct diameter is between about 500 μm and 2000 μm. In an embodiment, the implantable construct diameter is between about 500 μm and about 1900 μm, about 500 μm and about 1800 μm, about 500 μm and about 1700 μm, about 500 μm and about 1600 μm, about 500 μm and about 1500 μm, about 500 μm and about 1400 μm, about 500 μm and about 1300 μm, about 500 μm and about 1200 μm, about 500 μm and about 1100 μm, about 500 μm and about 1000 μm, about 500 μm and about 900 μm, about 500 μm and about 800 μm, about 500 μm and about 700 μm, or about 500 μm and about 600 μm. In an embodiment, the implantable construct diameter is between about 500 μm and about 1900 μm. In an embodiment, the implantable construct diameter is between about 500 μm and about 1800 μm. In an embodiment, the implantable construct diameter is between about 500 μm and about 1700 μm. In an embodiment, the implantable construct diameter is between about 500 μm and about 1600 μm. In an embodiment, the implantable construct diameter is between about 500 μm and about 1500 μm. In an embodiment, the implantable construct diameter is between about 500 μm and about 1400 μm. In an embodiment, the implantable construct diameter is between about 500 μm and about 1300 μm. In an embodiment, the implantable construct diameter is between about 500 μm and about 1200 μm. In an embodiment, the implantable construct diameter is between about 500 μm and about 1100 μm. In an embodiment, the implantable construct diameter is between about 500 μm and about 1000 μm. In an embodiment, the implantable construct diameter is between about 500 μm and about 900 μm. In an embodiment, the implantable construct diameter is between about 500 μm and about 800 μm. In an embodiment, the implantable construct diameter is between about 500 μm and about 700 μm. In an embodiment, the implantable construct diameter is between about 500 μm and about 600 μm.

In an embodiment, the implantable construct diameter is about 600 μm.

In an embodiment, the implantable construct diameter is between about 700 μm and 2000 μm. In an embodiment, the implantable construct diameter is between about 700 μm and about 1900 μm, about 700 μm and about 1800 μm, about 700 μm and about 1700 μm, about 700 μm and about 1600 μm, about 700 μm and about 1500 μm, about 700 μm and about 1400 μm, about 700 μm and about 1300 μm, about 700 μm and about 1200 μm, about 700 μm and about 1100 μm, about 700 μm and about 1000 μm, about 700 μm and about 900 μm, or about 700 μm and about 800 μm. In an embodiment, the implantable construct diameter is between about 700 μm and about 1900 μm. In an embodiment, the implantable construct diameter is between about 700 μm and about 1800 μm. In an embodiment, the implantable construct diameter is between about 700 μm and about 1700 μm. In an embodiment, the implantable construct diameter is between about 700 μm and about 1600 μm. In an embodiment, the implantable construct diameter is between about 700 μm and about 1500 μm. In an embodiment, the implantable construct diameter is between about 700 μm and about 1400 μm. In an embodiment, the implantable construct diameter is between about 700 μm and about 1300 μm. In an embodiment, the implantable construct diameter is between about 700 μm and about 1200 μm. In an embodiment, the implantable construct diameter is between about 700 μm and about 1100 μm. In an embodiment, the implantable construct diameter is between about 700 μm and about 1000 μm. In an embodiment, the implantable construct diameter is between about 700 μm and about 900 μm. In an embodiment, the implantable construct diameter is between about 700 μm and about 800 μm.

In an embodiment, the implantable construct diameter is about 800 μm.

In an embodiment, the implantable construct diameter is between about 1100 μm and 2000 μm. In an embodiment, the implantable construct diameter is between about 1100 μm and about 1900 μm, about 1100 μm and about 1800 μm, about 1100 μm and about 1700 μm, about 1100 μm and about 1600 μm, about 1100 μm and about 1500 μm, about 1100 μm and about 1400 μm, about 1100 μm and about 1300 μm, or about 1100 μm and about 1200 μm. In an embodiment, the implantable construct diameter is between about 1100 μm and about 1900 μm. In an embodiment, the implantable construct diameter is between about 1100 μm and about 1800 μm. In an embodiment, the implantable construct diameter is between about 1100 μm and about 1700 μm. In an embodiment, the implantable construct diameter is between about 1100 μm and about 1600 μm. In an embodiment, the implantable construct diameter is between about 1100 μm and about 1500 μm. In an embodiment, the implantable construct diameter is between about 1100 μm and about 1400 μm. In an embodiment, the implantable construct diameter is between about 1100 μm and about 1300 μm. In an embodiment, the implantable construct diameter is between about 1100 μm and about 1200 μm.

In an embodiment, the implantable construct diameter is about 1200 μm.

In an embodiment, the implantable construct diameter is between about 1600 μm and 2000 μm. In an embodiment, the implantable construct diameter is between about 1600 μm and about 1900 μm, about 1600 μm and about 1800 μm, or about 1600 μm and about 1700 μm. In an embodiment, the implantable construct diameter is between about 1600 μm and about 1900 μm. In an embodiment, the implantable construct diameter is between about 1600 μm and about 1800 μm. In an embodiment, the implantable construct diameter is between about 1600 μm and about 1700 μm.

In an embodiment, the implantable construct diameter is about 1700 μm.

In an embodiment, the implantable construct diameter is less than about 2000 μm. In an embodiment, the implantable construct diameter is less than about 1900 μm, about 1800 μm, about 1700 μm, about 1600 μm, about 1500 μm, about 1400 μm, about 1300 μm, about 1200 μm, about 1100 μm, about 1000 μm, about 900 μm, about 800 μm, about 700 μm, about 600 μm, about 500 μm, about 400 μm, about 300 μm, about 200 μm, or about 100 μm. In an embodiment, the implantable construct diameter is less than about 1900 μm. In an embodiment, the implantable construct diameter is less than about 1800 μm. In an embodiment, the implantable construct diameter is less than about 1700 μm. In an embodiment, the implantable construct diameter is less than about 1600 μm. In an embodiment, the implantable construct diameter is less than about 1500 μm. In an embodiment, the implantable construct diameter is less than about 1400 μm. In an embodiment, the implantable construct diameter is less than about 1300 μm. In an embodiment, the implantable construct diameter is less than about 1200 μm. In an embodiment, the implantable construct diameter is less than about 1100 μm. In an embodiment, the implantable construct diameter is less than about 1000 μm. In an embodiment, the implantable construct diameter is less than about 900 μm. In an embodiment, the implantable construct diameter is less than about 800 μm. In an embodiment, the implantable construct diameter is less than about 700 μm. In an embodiment, the implantable construct diameter is less than about 600 μm. In an embodiment, the implantable construct diameter is less than about 500 μm. In an embodiment, the implantable construct diameter is less than about 400 μm. In an embodiment, the implantable construct diameter is less than about 300 μm. In an embodiment, the implantable construct diameter is less than about 200 μm. In an embodiment, the implantable construct diameter is less than about 100 μm.

### Engineered Cells

Implantable constructs described herein may contain a cell, for example, an engineered cell. A cell can be derived from any mammalian organ or tissue, including the brain, nerves, ganglia, spine, eye, heart, liver, kidney, lung, spleen, bone, thymus, lymphatic system, skin, muscle, pancreas, stomach, intestine, blood, ovary, uterus, or testes.

A cell may be derived from a donor (e.g., an allogeneic cell), derived from a subject (e.g., an autologous cell), or from another species (e.g., a xenogeneic cell). In an embodiment, a cell can be grown in cell culture, or prepared from an established cell culture line, or derived from a donor (e.g., a living donor or a cadaver). In an embodiment, a cell is genetically engineered. In another embodiment, a cell is not genetically engineered. A cell may include a stem cell, such as a reprogrammed stem cell, or an induced pluripotent cell. Exemplary cells include mesenchymal stem cells (MSCs), fibroblasts (e.g., primary fibroblasts). HEK cells (e.g., HEK293T), Jurkat cells, HeLa cells, retinal pigment epithelial (RPE) cells, HUVEC cells, NIH3T3 cells, CHO-K1 cells, COS-1 cells, COS-7 cells, PC-3 cells, HCT 116 cells, A549MCF-7 cells, HuH-7 cells, U-2 OS cells, HepG2 cells, Neuro-2a cells, and SF9 cells. In an embodiment, a cell for use in an implantable construct is an RPE cell.

A cell included in an implantable construct may produce or secrete a therapeutic agent. In an embodiment, a cell included in an implantable construct may produce or secrete a single type of therapeutic agent or a plurality of therapeutic agents. In an embodiment, an implantable construct may comprise a cell that is transduced or transfected with a nucleic acid (e.g., a vector) comprising an expression sequence of a therapeutic agent. For example, a cell may be transduced or transfected with a lentivirus. A nucleic acid introduced into a cell (e.g., by transduction or transfection) may be incorporated into a nucleic acid delivery system, such as a plasmid, or may be delivered directly. In an embodiment, a nucleic acid introduced into a cell (e.g., as part of a plasmid) may include a region to enhance expression of the therapeutic agent and/or to direct targeting or secretion, for example, a promoter sequence, an activator sequence, or a cell-signaling peptide, or a cell export peptide. Exemplary promoters include EF-1a, CMV, Ubc, hPGK, VMD2, and CAG. Exemplary activators include the TET1 catalytic domain, P300 core, VPR, rTETR, Cas9 (e.g., from S. pyogenes or S. aureus), and Cpf1 (e.g., from L. bacterium).

An implantable construct described herein may comprise a cell or a plurality of cells. In an embodiment, the implantable construct is genetically engineered to produce or secrete a therapeutic agent. In an embodiment, the implantable construct comprises a cell producing or secreting a protein. The protein may be of any size, e.g., greater than about 100 Da, 200 Da, 250 Da, 500 Da, 750 Da, 1 kDa, 1.5 kDa, 2 kDa, 2.5 kDa, 3 kDa, 4 kDa, 5 kDa, 6 kDa, 7 kDa, 8 kDa, 9 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 125 kDa, 150 kDa, 200 kDa, 200 kDa, 250 kDa, 300 kDa, 400 kDa, 500 kDa, 600 kDa, 700 kDa, 800 Da, 900 kDa, or more. In an embodiment, the protein is composed of a single subunit or multiple subunits (e.g., a dimer, trimer, tetramer, etc.). A protein produced or secreted by a cell may be modified, for example, by glycosylation, methylation, or other known natural or synthetic protein modification. A protein may be produced or secreted as a pre-protein or in an inactive form and may require further modification to convert it into an active form.

Proteins produced or secreted by a cell may include antibodies or antibody fragments, for example, an Fc region or variable region of an antibody. Exemplary antibodies include anti-PD-1, anti-PD-L1, anti-CTLA4, anti-TNFα, and anti-VEGF antibodies. An antibody may be monoclonal or polyclonal. Other exemplary proteins include a lipoprotein, an adhesion protein, blood clotting factor (e.g., Factor VII, Factor VIII, Factor IX, GCG, or VWF), hemoglobin, enzymes, proenkephalin, a growth factor (e.g., EGF, IGF-1, VEGF alpha, HGF, TGF beta, bFGF), or a cytokine.

A protein produced or secreted by a cell may include a hormone. Exemplary hormones include growth hormone, growth hormone releasing hormone, prolactin, lutenizing hormone (LH), anti-diuretic hormone (ADH), oxytocin, thyroid stimulating hormone (TSH), thyrotropin-release hormone (TRH), adrenocorticotropic hormone (ACTH), follicle-stimulating hormone (FSH), thyroxine, calcitonin, parathyroid hormone, aldosterone, cortisol, epinephrine, glucagon, insulin, estrogen, progesterone, and testosterone.

A protein produced or secreted by a cell may include a cytokine. In an embodiment, the cytokine is a cytokine of the IL-12 family, e.g., IL-12, IL-23, IL-27, or IL-35.

A cytokine may be a pro-inflammatory cytokine. Exemplary proinflammatory cytokines include interferon gamma (IFNγ), interleukin-1 alpha (IL-1α), interleukin-1 beta (IL-1β), interleukin-2 (IL-2), interleukin-1 beta (IL-1β), interleukin-6 (IL-6), interleukin-8 (IL-8), interleukin-12 (IL-12), interleukin-17A (IL-17A), interleukin 18 (IL-18), interleukin-23 (IL-23) interleukin-33 (IL-33), interleukin-36 alpha (IL-36α), interleukin-36 beta (IL-36β), interleukin-36 gamma (IL-36γ), tumor necrosis factor alpha (TNFα), and tumor necrosis factor alpha (TNFα). Exemplary anti-inflammatory cytokines include interferon beta (IFNβ), interleukin-2 (IL-2), interleukin 4 (IL-4), interleukikn-5 (IL-5) interleukin-6 (IL-6), interleukin-10 (IL-10), interleukin-13 (IL-13), interleukin-19 (IL-19), interleukin-27 (IL-27), interleukin-33 (IL-33), interleukin-35 (IL-35), interleukin-37 (IL-36), interleukin-38 (IL-38), and transforming growth factor beta (TGFβ).

A cytokine may be an immunomodulatory cytokine. Exemplary immunomodulatory cytokines include interferon alpha (IFNα), interleukin-1 receptor antagonist protein (IL-1Ra), interleukin-27 (IL-27), and interleukin-36 receptor antagonist (IL-36Ra).

The engineered cell is further contemplated in some embodiments to secrete a chemokine. Exemplary proinflammatory chemokines include chemokine (C—C motif) ligand 2 (CCL2), chemokine (C—C motif) ligand 3, (CCL3), chemokine (C—C motif) ligand 5 (CCL5), chemokine (C—C motif) ligand 20 (CCL20), chemokine (C—X—C motif) ligand 1 (CXCL1), chemokine (C—X—C motif) ligand 4 (CXCL4), chemokine (C—X—C motif) ligand 5 (CXCL5), chemokine (C—X—C motif) ligand 6 (CXCL6), chemokine (C—X—C motif) ligand 7 (CXCL7), chemokine (C—X—C motif) ligand 8 (CXCL8), chemokine (C—X—C motif) ligand 9 (CXCL9), and chemokine (C—X—C motif) ligand 10 (CXCL10).

A chemokine may be anti-inflammatory. Exemplary anti-inflammatory chemokines include (C—C motif) ligand 17 (CCL17), chemokine (C—C motif) ligand 18 (CCL18), chemokine (C—C motif) ligand 19 (CCL19), chemokine (C—C motif) ligand 21 (CCL21), chemokine (C—X—C motif) ligand 12 (CXCL12), chemokine (C—X—C motif) ligand 13 (CXCL13), and chemokine (C—X—C motif) ligand 16 (CXCL16).

A cytokine may include any cytokine described in M. J. Cameron and D. J. Kelvin, Cytokines, Chemokines, and Their Receptors (2013), Landes Biosciences, which is incorporated herein by reference in its entirety.

A implantable construct may comprise a cell expressing a single type of therapeutic agent, e.g., a single protein or nucleic acid, or may express more than one type of therapeutic agent, e.g., a plurality of proteins or nucleic acids. In an embodiment, a implantable construct comprises a cell expressing two types of therapeutic agents (e.g., two types of proteins or nucleic acids). In an embodiment, a implantable construct comprises a cell expressing three types of therapeutic agents (e.g., three types of proteins or nucleic acids). In an embodiment, an implantable construct comprises a cell expressing four types of therapeutic agents (e.g., four types of proteins or nucleic acids).

In an embodiment, an implantable construct comprises a cell expressing a single type of nucleic acid (e.g., DNA or RNA) or may express more than one type of nucleic acid, e.g., a plurality of nucleic acid (e.g., DNA or RNA). In an embodiment, an implantable construct comprises a cell expressing two types of nucleic acids (e.g., DNA or RNA). In an embodiment, an implantable construct comprises a cell expressing three types of nucleic acids (e.g., DNA or RNA). In an embodiment, an implantable construct comprises a cell expressing four types of nucleic acids (e.g., DNA or RNA).

In an embodiment, an implantable construct comprises a cell expressing a single type of protein, or may express more than one type of protein, e.g., a plurality of proteins. In an embodiment, an implantable construct comprises a cell expressing two types of proteins. In an embodiment, an implantable construct comprises a cell expressing three types of proteins. In an embodiment, an implantable construct comprises a cell expressing four types of proteins.

In an embodiment, an implantable construct comprises a cell expressing a single type of enzyme, or may express more than one type of enzyme, e.g., a plurality of enzymes. In an embodiment, an implantable construct comprises a cell expressing two types of enzymes. In an embodiment, an implantable construct comprises a cell expressing three types of enzymes. In an embodiment, an implantable construct comprises a cell expressing four types of enzymes.

In an embodiment, an implantable construct comprises a cell expressing a single type of antibody or antibody fragment or may express more than one type of antibody or antibody fragment, e.g., a plurality of antibodies or antibody fragments. In an embodiment, an implantable construct comprises a cell expressing two types of antibodies or antibody fragments. In an embodiment, an implantable construct comprises a cell expressing three types of antibodies or antibody fragments. In an embodiment, an implantable construct comprises a cell expressing four types of antibodies or antibody fragments.

In an embodiment, an implantable construct comprises a cell expressing a single type of hormone, or may express more than one type of hormone, e.g., a plurality of hormones. In an embodiment, an implantable construct comprises a cell expressing two types of hormones. In an embodiment, an implantable construct comprises a cell expressing three types of hormones. In an embodiment, an implantable construct comprises a cell expressing four types of hormones.

In an embodiment, an implantable construct comprises a cell expressing a single type of enzyme, or may express more than one type of enzyme, e.g., a plurality of enzymes. In an embodiment, an implantable construct comprises a cell expressing two types of enzymes. In an embodiment, an implantable construct comprises a cell expressing three types of enzymes. In an embodiment, an implantable construct comprises a cell expressing four types of enzymes. In an embodiment, an implantable construct comprises a cell expressing a single type of cytokine or may express more than one type of cytokine, e.g., a plurality of cytokines. In an embodiment, an implantable construct comprises a cell expressing two types of cytokines. In an embodiment, an implantable construct comprises a cell expressing three types of cytokines. In an embodiment, an implantable construct comprises a cell expressing four types of cytokines.

In the case of a plurality of cells, the concentration and total cell number may be varied depending on a number of factors, such as cell type, implantation location, and expected lifetime of the implantable construct. In an embodiment, the total number of cells included in an implantable construct is greater than about 2, 4, 6, 8, 10, 20, 30, 40, 50, 75, 100, 200, 250, 500, 750, 1000, 1500, 2000, 5000, 10000, or more. In an embodiment, the total number of cells included in an implantable construct is greater than about 1.0×10², 1.0×10³, 1.0×10⁴, 1.0×10⁵, 1.0×10⁶, 1.0×10⁷, 1.0×10⁸, 1.0×10⁹, 1.0×10¹¹, or more. In an embodiment, the total number of cells included in an implantable construct is less than about than about 10000, 5000, 2500, 2000, 1500, 1000, 750, 500, 250, 200, 100, 75, 50, 40, 30, 20, 10, 8, 6, 4, 2, or less. In an embodiment, the total number of cells included in an implantable construct is less than about 1.0×10¹⁰, 1.0×10⁹, 1.0×10⁸, 1.0×10⁷, 1.0×10⁶, 1.0×10⁵, 1.0×10⁴, 1.0×10³, 1.0×10², or less. In an embodiment, a plurality of cells is present as an aggregate. In an embodiment, a plurality of cells is present as a cell dispersion.

Specific features of a cell contained within an implantable construct may be determined, e.g., prior to and/or after incorporation into the implantable construct. For example, cell viability, cell density, or cell expression level may be assessed. In an embodiment, cell viability, cell density, and cell expression level may be determined using standard techniques, such as cell microscopy, fluorescence microscopy, histology, or biochemical assay.

### Gene Autoregulation of Therapeutic Agent Expression

It is contemplated that in instances where the engineered cell expresses a therapeutic agent, e.g., a cytokine, it is desirable that the level of production of the therapeutic agent be auto regulated in order to prevent secretion of toxic levels of the cytokine. One way to accomplish this is to introduce an operator site into the DNA region between the cytokine gene and its promoter in a first ORF. A second ORF is used that encodes a transcriptional repressor that binds to the operator site under the control of a promoter that is activated as a result of signaling through the cytokine's receptor. In this way, the cells can sense the cytokine in their environment and reduce their production of the cytokine when there is sufficient cytokine already present.

Another possible strategy is to introduce a sequence that forms a higher-order structure into the 5′ untranslated region (5′ UTR) of the cytokine gene. Then a second ORF is
used that encodes an RNA-binding protein that binds to the higher-order structure, and
suppresses translation, under the control of a promoter that is activated as a result of signaling through the cytokine's receptor.

Another possible strategy is to introduce several repeats of a synthetic microRNA

(miRNA) target site into the 3′ untranslated region (3′ UTR) of the cytokine gene. Then a secondORF is used that encodes the miRNA under the control of a promoter that is activated as a result of signaling through the cytokine's receptor.

Another possible strategy is to use a second ORF encoding a synthetic ubiquitin ligase that targets the cytokine, and leads to ubiquitin-mediated proteolysis, under the control of a promoter that is activated as a result of signaling through the cytokine's receptor. In this case, the cytokine gene may be modified to include additional protein domains if doing so is necessary in order to make the cytokine recognizable by the synthetic ubiquitin ligase. Ideally, the addition of any additional protein domains will not alter the cytokine's immunological functions.

A further strategy is to incorporate a mechanism where the therapeutic agent is under control of a promoter, e.g., a synthetic promoter or a natural promoter), which binds to a native transcription factor whose activation is downstream of a receptor, e.g., a cytokine receptor, e.g., NF-κB or NFAT.

These self-regulated control strategies could be combined with small molecule-based strategies to provide an additional level of control to the cytokine production. Using a small molecule-activated promoter (such as the TRE/tetracycline system) to drive expression of the cytokine would allow for external regulation of the cytokine production by the administration of the small molecule. Post-transcriptional control of the cytokine expression is also possible using small molecule-dependent riboswitches—a short sequence could be added to the 5′ or 3′ UTR of the cytokine gene that forms a small molecule-dependent functional higher-order structure, such as a frame-shifting aptamer or a mRNA-cleaving aptazyme, allowing for similar external control of the cytokine production, since there are examples of these systems that turn on frame-shifting or cleavage upon the addition of a small molecule and examples that turn off in the presence of the small molecule. This type of control is also possible at the protein level by adding the sequence for a destabilization domain that can be stabilized by a small molecule to the beginning or end of the gene for the cytokine, which would lead to targeted degradation of the cytokine whenever the small molecule is not present. The reverse is also possible by augmenting the gene for the cytokine with the sequence for a small molecule-assisted shutoff (SMASh) system, which includes a destabilization domain and a non-mammalian protease that cleaves the destabilization domain from the cytokine except in the presence of a small molecule protease inhibitor that would prevent cleavage and lead to degradation of the cytokine. All these modifications to the protein structure could also be done indirectly by instead modifying a synthetic transcription factor that activates the promoter controlling expression of the cytokine, which would ensure that all these protein modifications stay within the therapeutic cells instead of being secreted and potentially generating an immune response to these unnatural protein domains. One possible synthetic transcription factor to use for this purpose is a fusion between the transcriptional activators VP64, p65, and Rta (VPR) and catalytically inactivated Cas9 (dCas9), which when coexpressed with a guide RNA (gRNA) will localize the VPR complex to the synthetic promoter with complementarity to the gRNA in order to activate transcription of the cytokine gene.

### Controlling Drug Delivery and Release Kinetics

The vector systems contemplated in the engineered cells may further comprise a kill switch to arrest the therapy, similar to the kill switch designed for CAR T cells. For example, two engineered proteins may be located inside the encapsulated cells, that dimerize when exposed to a small molecule drug called rimiducid. This drug activates a protein called caspase-9, which induces cell death.

Chemical Induction of Dimerization (CID) with small molecules is an effective technology used to generate switches of protein function to alter cell physiology. A high specificity, efficient dimerizer is rimiducid (AP1903), which has two identical, protein-binding surfaces arranged tail-to-tail, each with high affinity and specificity for a mutant or variant of FKBP12: FKBP12 (F36V) (FKBP12v36, FV36 or FV). Attachment of one or more FV domains onto one or more cell signaling molecules that normally rely on homodimerization can convert that protein to rimiducid control. For example, a molecular switch is provided that provides the option to activate a pro-apoptotic polypeptide, such as, for example, Caspase-9, with rimiducid, wherein the chimeric pro-apoptotic polypeptide comprises a rimiducid-induced switch. In one embodiment of the switch technology, a homodimerizer, such as AP1903 (rimiducid), activates a safety switch, causing apoptosis of the modified cell. In this embodiment, for example, a chimeric pro-apoptotic polypeptide, such as, for example, Caspase-9, comprising a FKBP12 multimerizing region is expressed in a cell. Upon contacting the cell with a dimerizer that binds to the Fv regions, the chimeric polypeptide dimerizes or multimerizes, and activates the cell. The cell may, for example, be an engineered cell that expresses a protein, e.g., a cytokine.

Furthermore, a transmembrane sensor can be engineered into the engineered cells to create a feedback loop to regulate output of the therapeutic agent. The transmembrane sensor responds to varying concentrations of the protein of interest and uses a positive or negative feedback loop to suppress the transcription of the therapeutic agent of interest, e.g., cytokine or chemokine, with the help of an inducible promoter. This allows fine-tuning of the localized delivery of the protein of interest and ensures that there is no over-expression of the protein of interest. The alginate biomaterial used allows for rapid diffusion across the inner and outer shell to give real-time feedback to this sense-and-respond genetic cellular circuit.

### Features of Implantable Constructs

The implantable construct described herein may take any suitable shape or morphology. For example, an implantable construct may be a sphere, spheroid, tube, cord, string, ellipsoid, disk, cylinder, sheet, torus, cube, stadiumoid, cone, pyramid, triangle, rectangle, square, or rod. An implantable construct may comprise a curved or flat section. In an embodiment, an implantable construct may be prepared through the use of a mold, resulting in a custom shape.

The implantable construct may vary in size, depending, for example, on the use or site of implantation. For example, an implantable construct may have a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. In an embodiment, an implantable construct may have a section or region with a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. In an embodiment, an implantable construct may have a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller. In an embodiment, an implantable construct may have a section or region with a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller.

In an embodiment, an implantable construct comprises a pore or opening to permit passage of an object, such as a small molecule (e.g., nutrients or waste), a protein, or a nucleic acid. For example, a pore in or on an implantable construct may be greater than 0.1 nm and less than 10 μm. In an embodiment, the implantable construct comprises a pore or opening with a size range of 0.1 μm to 10 μm, 0.1 μm to 9 μm, 0.1 μm to 8 μm, 0.1 μm to 7 μm, 0.1 μm to 6 μm, 0.1 μm to 5 μm, 0.1 μm to 4 μm, 0.1 μm to 3 μm, 0.1 μm to 2 μm.

An implantable construct described herein may comprise a chemical modification in or on any enclosed material. Exemplary chemical modifications include small molecules, peptides, proteins, nucleic acids, lipids, or oligosaccharides. The implantable construct may comprise at least 0.5%, 1%, 2%, 3%, 4%, 5%, 7.5%, 10%, 15%, 20%, 30%, 40%, 50%, 60%, 70%, 80% or more of a material that is chemically modified, e.g., with a chemical modification described herein. An implantable construct may be partially coated with a chemical modification, e.g., at least about 5%, 10%, 15%, 20%, 25%, 30%, 35%, 40%, 45%, 50%, 55%, 60%, 65%, 70%, 75%, 80%, 85%, 90%, 95%, 99%, or 99.9% coated with a chemical modification.

In some embodiments, the implantable construct is chemically modified with a specific density of modifications. The specific density of chemical modifications may be described as the average number of attached chemical modifications per given area. For example, the density of a chemical modification on or in an implantable construct may be 0.01, 0.1, 0.5, 1, 5, 10, 15, 20, 50, 75, 100, 200, 400, 500, 750, 1,000, 2,500, or 5,000 chemical modifications per square μm or square mm.

An implantable construct may be formulated or configured for implantation in any organ, tissue, cell, or part of a subject. For example, the implantable construct may be implanted or disposed into the intraperitoneal space of a subject. An implantable construct may be implanted in or disposed on a tumor or other growth in a subject, or be implanted in or disposed about 0.1 mm, 0.5 mm, 1 mm, 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, 1 cm, 5, cm, 10 cm, or further from a tumor or other growth in a subject. A implantable construct may be configured for implantation, or implanted, or disposed on or in the skin, a mucosal surface, a body cavity, the central nervous system (e.g., the brain or spinal cord), an organ (e.g., the heart, eye, liver, kidney, spleen, lung, ovary, breast, uterus), the lymphatic system, vasculature, oral cavity, nasal cavity, gastrointestinal tract, bone, muscle, adipose tissue, skin, or other area.

An implantable construct may be formulated for use for any period of time. For example, an implantable construct may be used for 1 hour, 2 hours, 4 hours, 6 hours, 12 hours, 1 day, 36 hours, 2 days, 3 days, 4 days, 5 days, 6 days, 1 week, 2 weeks, 3 weeks, 4 weeks, 5 weeks, 6 weeks, 2 months, 3 months, 4 months, 5 months, 6 months, 1 year, or longer. An implantable construct can be configured for limited exposure (e.g., less than 2 days, e.g., less than 2 days, 1 day, 24 hours, 20 hours, 16 hours, 12 hours, 10 hours, 8 hours, 6 hours, 5 hours, 4 hours, 3 hours, 2 hours, 1 hour or less). An implantable construct can be configured for prolonged exposure (e.g., at least 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more). An implantable construct can be configured for permanent exposure (e.g., at least 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more).

In an embodiment, an implantable construct comprising a plurality of engineered cells is capable of producing a cytokine a production rate of 0.1, 1, 2, 3, 4, 5, 6, 7, 8, 9, 10, 12, 14, 16, 18, 20, 22, 24, 26, 28, 30, 50, or 100 ng/mL/day/implantable construct. In an embodiment, the cytokine is IL-2mt, IL-10, IL-12, IL-23, IL-27, or IL-35 or a functional variant thereof. In an embodiment, 1.5-mm implantable constructs comprising ARPE-19 cells capable of expressing IL-10 (RPE-IL10) exhibit a production rate of about 6.4 ng/ml/day/implantable construct after 24-h incubation as evaluated by a protein-specific ELISA. In an embodiment, 1.5-mm implantable constructs comprising ARPE-19 cells capable of expressing IL-12 (RPE-IL12) exhibit a production rate of about 25.7 ng/ml/day/implantable construct after 24-h incubation as evaluated by a protein-specific ELISA. In an embodiment, 1.5-mm implantable constructs comprising ARPE-19 cells capable of expressing IL-2mt (RPE-IL2mt) exhibit a production rate of about 26.2 ng/ml/day/implantable construct after 24-h incubation as evaluated by a protein-specific ELISA.

In an embodiment, an implantable construct comprising a plurality of engineered cells is capable of producing a cytokine a production rate of 100, 200, 300, 400, 500, 600, 700, 800, 900, 1000, 1200, 1400, 1600, 1800, 2000, 2500, 3000, 3500, 4000, 5000 pg/mL/day/implantable construct in a body cavity after local administration in a subject, i.e., a patient. in an embodiment, the body cavity is the intraperitoneal (IP) cavity, i.e., IP space. In an embodiment, the cytokine is IL-2mt, IL-10, IL-12, IL-23, IL-27, or IL-35 or a functional variant thereof. In an embodiment, the production of RPE-IL10 is about 200 pg/mL in the intraperitoneal (IP) cavity, as measured one month after local administration of implantable constructs comprising a plurality of engineered RPE-IL10 implantable constructs. In an embodiment, the production of IL-12 is between about 2000 to about 3000 pg/mL in the intraperitoneal (IP) cavity, as measured one month after local administration of RPE-IL12 implantable constructs. In an embodiment, the production of IL-2mt is between about 50 to about 100 pg/mL in the intraperitoneal (IP) cavity, as measured one month after local administration of RPE-IL2mt implantable constructs. In an embodiment, the production of IL-10 is about 0 pg/mL in blood, as measured one month after local administration of RP-IL10 implantable constructs. In an embodiment, the production of IL-12 is less than 1000 pg/mL in blood, as measured one month after local administration of RPE-IL12 implantable constructs. In an embodiment, the production of IL-2mt is about 0 pg/mL in blood, as measured one month after local administration of RPE-IL2mt implantable constructs.

In an embodiment, fibrosis on the surface of an implantable construct abrogated by 1%, 5%, 10%, 20%, 30%, 40%, 50% or more after local administration of implantable constructs into a body cavity of a subject, i.e., a patient, 1 week, 2 weeks, 3 weeks, 4 weeks, 1 month, 2 months or 3 months after administration relative to a control group. In an embodiment, the implantable construct comprises a plurality of engineered cells capable of producing a cytokine. In an embodiment, the cytokine is IL-2mt, IL-10, IL-12, IL-23, IL-27, or IL-35 or a functional variant thereof. In an embodiment, the diameter of the implantable construct is 1.5 mm. In an embodiment, the engineered cell is an RPE cell, e.g., ARPE-19 cell. In an embodiment, the cavity is the intraperitoneal (IP) space. In an embodiment, fibrosis on the implantable construct surface is abrogated in 1.5-mm diameter RPE-IL10 implantable constructs explanted one month after implantation in the IP space relative to non-engineered and RPE-IL-2mt implantable constructs. In an embodiment, fibrosis is abrogated in 1.5-mm diameter RPE-IL12 implantable constructs explanted one month after implantation in the IP space relative to non-engineered ARPE-19-containing or RPE-IL2mt implantable constructs.

In an embodiment, expression of one or more fibrotic markers is reduced after local administration of an implantable construct one month after local administration into a body cavity in a subject, i.e., a patient relative to a control group. In an embodiment, the implantable construct comprises a plurality of engineered cells capable of producing a cytokine. In an embodiment, the fibrotic marker is αSMA, Col1a1, Col1a2 or a combination thereof. In an embodiment, the expression of fibrotic markers is measured by RT-qPCR. In an embodiment, the cytokine is IL-2mt, IL-10, IL-12, IL-23, IL-27, or IL-35 of a functional variant thereof. In an embodiment, the diameter of the implantable construct is 1.5 mm. In an embodiment, the engineered cell is an RPE cell, e.g., ARPE-19 cell. In an embodiment, expression of one or more fibrotic markers e.g., alpha-smooth muscle actin (aSMA) or CD68, on the implantable construct surface is reduced in 1.5-mm diameter RPE-IL10 implantable constructs explanted one month after implantation in the IP space relative to non-engineered ARPE-19-containing or RPE-IL-2mt implantable constructs. In an embodiment, fibrosis is prevented on 1.5-mm diameter RPE-IL10 implantable constructs explanted one month after implantation in the IP space relative to non-engineered ARPE-19-containing or RPE-IL-2mt implantable constructs. 1.5-mm diameter RPE-IL10 implantable constructs explanted one month after implantation in the IP space relative to non-engineered ARPE-19-containing or RPE-IL-2mt implantable constructs. In an embodiment, expression of one or more fibrotic markers e.g., alpha-smooth muscle actin (aSMA) or CD68, on the implantable construct surface is reduced in 1.5-mm diameter RPE-IL12 cells explanted one month after implantation in the IP space relative to non-engineered ARPE-19-containing or RPE-IL-2mt implantable constructs.

In an embodiment, the expression of fibrosis-related genes, e.g., αSMA, Col1a1, and/or Col1a2, as measured by RT-qPCR, as quantified from the cells of the 1.5-mm diameter RPE-IL 10 implantable constructs explanted one month after implantation in the IP space, is reduced relative to non-engineered ARPE-19-containing or RPE-IL-2mt implantable constructs. In an embodiment, the expression of fibrosis-related genes, e.g., αSMA, Col1a1, and/or Col1a2, as measured by RT-qPCR, as quantified from the cells of the 1.5-mm diameter RPE-IL12 implantable constructs explanted one month after implantation in the IP space relative to non-engineered ARPE-19-containing or RPE-IL-2mt implantable constructs.

In an embodiment, there is a measurable reduction in fibrotic deposition after local administration of an implantable construct one month after local administration into a body cavity in a subject, i.e., a patient relative to a control group. In an embodiment, the implantable construct comprises a plurality of engineered cells capable of producing a cytokine. In an embodiment, the cytokine is IL-2mt, IL-10, IL-12, IL-23, IL-27, or IL-35 or a functional variant thereof. In an embodiment, the cavity is the intraperitoneal (IP) space. In an embodiment, the diameter of the implantable construct is 1.5 mm. In an embodiment, the engineered cell is an RPE cell, e.g., ARPE-19 cell. In an embodiment, the surfaces of RPE-IL-10 implantable constructs and the empty microcapsules have a measurable reduction in fibrotic deposition relative to a control group, e.g., empty implantable constructs or RPE implantable constructs (i.e., not capable of secreting a cytokine), three months after implantation in the intraperitoneal (IP) space. In an embodiment, the surfaces of RPE-IL-10 implantable constructs have a measurable reduction in fibrotic deposition relative to a control group, e.g., empty implantable constructs or RPE implantable constructs (i.e., not capable of secreting a cytokine), three months after implantation in the intraperitoneal (IP) space.

In an embodiment, the surfaces of RPE-IL-12 implantable constructs have a measurable reduction in fibrotic deposition relative to a control group, e.g., empty implantable constructs or RPE implantable constructs (i.e., not capable of secreting a cytokine), three months after implantation in the intraperitoneal (IP) space. In an embodiment, the surfaces of RPE-IL-12 implantable constructs have a measurable reduction in fibrotic deposition relative to a control group, e.g., empty implantable constructs or RPE implantable constructs (i.e., not capable of secreting a cytokine), three months after implantation in the intraperitoneal (IP) space.

In an embodiment, cytokines do not systemically accumulate, e.g., accumulate in the blood or circulatory system, in a subject, i.e., a patient after local administration of an implantable construct one month after local administration into a body cavity in a subject, i.e., a patient relative to a control group. In an embodiment, the implantable construct comprises a plurality of engineered cells capable of producing a cytokine. In an embodiment, the cytokine is IL-2mt, IL-10, IL-12, IL-23, IL-27, or IL-35 or a functional variant thereof. In an embodiment, the cavity is locally administered in the intraperitoneal (IP) space. In an embodiment, the diameter of the implantable construct is 1.5 mm. In an embodiment, the engineered cell is an RPE cell, e.g., ARPE-19 cell. In an embodiment, RPE-IL10 implantable constructs produce IL-10 in the IP space for at least six months after local administration. In an embodiment, RPE-IL12 implantable constructs produce IL-12 in the IP space for at least six months after local administration. In an embodiment, IL-10 does not systemically accumulate (e.g., accumulate in blood) for at least six months after administration of RPE-IL10 implantable constructs. In an embodiment, IL-12 does not systemically accumulate (e.g., accumulate in blood) for at least six months after administration of RPE-IL12 implantable constructs.

In an embodiment, immune cells accumulate more readily at the implantable construct boundary and in the spleen after local administration in a subject, i.e., a patient, relative to a control group. In an embodiment, the implantable construct comprises a plurality of engineered cells capable of producing a cytokine. In an embodiment, the cytokine is IL-2mt, IL-10, IL-12, IL-23, IL-27, or IL-35 or a functional variant thereof. In an embodiment, the immune cells are granulocytes, monocytes (e.g., macrophages), dendritic cells, T cells, natural killer (NK) cells, or a combination thereof. In an embodiment, the implantable constructs are administered locally to the intraperitoneal (IP) space. In an embodiment the accumulation is measured by uniform manifold approximation and projection (UMAP). In an embodiment, granulocytes, monocytes (e.g., macrophages), dendritic cells, T cells, and natural killer (NK) cells accumulate at the RPE-IL10 implantable construct boundary and in the spleen after local administration in the intraperitoneal space in a subject, i.e., a patient, as measured by uniform manifold approximation and projection (UMAP). In an embodiment, granulocytes, monocytes (e.g., macrophages), dendritic cells, T cells and natural killer (NK) cells accumulate at the RPE-IL 12 implantable construct boundary and in the spleen after local delivery in the intraperitoneal (IP) space in a rodent model as measured by uniform manifold approximation and projection (UMAP). In an embodiment, the proportion of monocytes increases by 1.4-fold (Fisher exact test p=0.047) relative to the Sham group in the immune infiltrate after implantation of implantable constructs capable of expressing IL-10 or IL-12 in the intraperitoneal (IP) space in a rodent model. In an embodiment, the proportion of granulocytes decreases by 3-fold (Fisher exact test p=0.049) relative to the Sham group at the RPE-IL12 implantable construct boundary after implantation in the intraperitoneal (IP) space in a rodent model.

In an embodiment, a cytokine, e.g., IL-10, IL-12, IL-27, or IL-35 prevent fibrosis. In an embodiment, the cytokine prevents fibrosis by suppressing inflammation. In an embodiment, the cytokine prevents fibrosis by inhibiting the TGFβ pathway. In an embodiment, Uniform Manifold Approximation and Projection (UMAP) embedding indicates that cell-type specific markers correspond to granulocytes, monocytes, e.g., macrophages, dendritic cells, B cells, T cells, and natural killer cells upon implantation of an implant construct comprising engineered cells capable of producing a cytokine, e.g., IL-10, IL-12, IL-27, or IL-35. In an embodiment, the proportion of proportion of granulocytes, T cells, and NK cells is reduced by at least 3% upon implantation of an implant construct comprising engineered cells capable of producing a cytokine e.g., IL-10, IL-12, IL-27, or IL-35. In an embodiment, the proportion of B cells and dendritic cells is decreased by 3% or lesser upon implantation of an implant construct comprising engineered cells capable of producing a cytokine, e.g., IL-10, IL-12, IL-27, or IL-35.

In an embodiment, implantable constructs, e.g., RPE-IL10- and RPE-IL12 implantable constructs, enrich the population of macrophage-containing monocytes upon implantation. In an embodiment, RPE-10 has suppresses selections of genes associated with the inflammatory response, comprising allograft rejection with a normalized enrichment score of NES of about −1.74 and antigen processing with an NES of about-1.74, upon implantation. In an embodiment, RPE-IL10 implantable constructs result in a downregulation of inflammatory cytokines Cxcl19 expression in monocytes. In an embodiment, RPE-IL-10 implantable constructs exert effects on the fibrotic TGFβ pathway with a NES of about-0.97.

In an embodiment, RPE-IL12 implantable constructs promote the inflammatory response upon implantation. In an embodiment, RPE-IL12 implantable constructs promote the allograft rejection with an NES of about 1.32 upon implantation. In an embodiment, RPE-IL12 implantable constructs promote the antigen processing cross-presentation with an NES of about 1.72 upon implantation. In an embodiment, RPE-IL12 implantable constructs inhibit the TGFβ pathway with an NES of about-2.1 upon implantation. In an embodiment, RPE-IL12 implantable constructs downregulate a profibrotic cytokine, e.g., Tgfb2.

In an embodiment, normal glycemic control is maintained for 25, 30, 35, 40, 45, 50, 60, 70, 80, 90, 100 days or more after local administration of an implantable construct into a body cavity of a subject, i.e., a patient. In an embodiment, the implantable construct comprises a plurality of engineered cells capable of producing a cytokine. In an embodiment, normal glycemic control is maintained for 50 days or more after local administration of an implantable construct into a body cavity of a subject, i.e., a patient, wherein the implantable construct comprises a plurality of engineered cells capable of producing a cytokine. In an embodiment, the cytokine is IL-2mt, IL-10, IL-12, IL-23, IL-27, or IL-35 or a functional variant thereof. In an embodiment, the cavity is the intraperitoneal (IP) space. In an embodiment, the diameter of the implantable construct is 1.5 mm. In an embodiment, the engineered cell is an RPE cell, e.g., ARPE-19 cell. In an embodiment, the blood glucose concentration is maintained below 250 mg/dL. In an embodiment, streptozotocin (STZ)-induced diabetic C57BL/6J mice maintain normal glycemic control for 50 days or more after administration of RPE-IL10 implantable constructs to the intraperitoneal (IP) space relative to a Control group, e.g., an RPE group (i.e., not capable of secreting a cytokine). In an embodiment, streptozotocin (STZ)-induced diabetic C57BL/6J mice maintain normal glycemic control for 50 days or more after administration of RPE-IL12 implantable constructs to the intraperitoneal (IP) space relative to a Control group, e.g., an RPE group. In an embodiment, streptozotocin (STZ)-induced diabetic C57BL/6J mice maintain a blood glucose concentration below 250 mg/dL for 50 days or more after administration of RPE-IL10 implantable constructs to the intraperitoneal (IP) space. In an embodiment, streptozotocin (STZ)-induced diabetic C57BL/6J mice maintain a blood glucose concentration below 250 mg/dL for 50 days or more after administration of RPE-IL12 implantable constructs to the intraperitoneal (IP) space.

In an embodiment, normal glycemic control is maintained for 100 days or more after local administration of an implantable construct into a body cavity of a subject, i.e., a patient. In an embodiment, the implantable construct comprises a plurality of engineered cells capable of producing a cytokine. In an embodiment, the cytokine is IL-2mt, IL-10, IL-12, IL-23, IL-27, or IL-35 or a functional variant thereof. In an embodiment, the cavity is the intraperitoneal (IP) space. In an embodiment, the diameter of the implantable construct is 1.5 mm. In an embodiment, the engineered cell is an RPE cell, e.g., ARPE-19 cell. In an embodiment, the blood glucose concentration is maintained below 250 mg/dL. In an embodiment, streptozotocin (STZ)-induced diabetic C57BL/6J mice maintain normal glycemic control for 100 days or more after administration of RPE-IL10 implantable constructs to the intraperitoneal (IP) space relative to a Control group, e.g., an islet cap group.

In an embodiment, the production of a cytokine is localized to the administration site, i.e., , body cavity, after about 100 days or longer after local administration of the implantable construct into the body cavity of a subject, relative to a control group. In an embodiment, the implantable construct comprises a plurality of engineered cells capable of producing a cytokine. In an embodiment, the cytokine is IL-2mt, IL-10, IL-12, IL-23, IL-27, or IL-35 or a functional variant thereof. In an embodiment, the cavity is the intraperitoneal (IP) space. In an embodiment, the diameter of the implantable construct is 1.5 mm. In an embodiment, the engineered cell is an RPE cell, e.g., ARPE-19 cell. In an embodiment, the production of IL-10 is between about 100 pg/mL and 150 pg/mL in the IP fluid about 100 days after implantation of RPE-IL10 implantable constructs in streptozotocin (STZ)-induced C57BL/6J diabetic mice. In an embodiment, the production of IL-10 is about 0 pg/mL in the blood 100 days after implantation of RPE-IL10 implantable constructs in streptozotocin (STZ)-induced C57BL/6J diabetic mice.

In an embodiment, fibrotic overgrowth on the surface of the implantable constructs reduced by 5%, 10%, 15%, 20%, 25%, 30%, 35%, 40%, 50%, or more relative to a control group. In an embodiment, the implantable construct comprises a plurality of engineered cells capable of producing a cytokine. In an embodiment, the cytokine is IL-2mt, IL-10, IL-12, IL-23, IL-27, or IL-35 or a functional variant thereof. In an embodiment, the cavity is the intraperitoneal (IP) space. In an embodiment, the diameter of the implantable construct is 1.5 mm. In an embodiment, the engineered cell is an RPE cell, e.g., ARPE-19 cell. In an embodiment, the fibrotic overgrowth on the surface of RPE-IL10 implantable constructs explanted from streptozotocin (STZ)-induced C57BL/6J diabetic mice is reduced after administration of the RPE-IL10 implantable constructs in the intraperitoneal (IP) space relative to a control group, e.g., RPE or islet cap groups.

In an embodiment, c-peptide concentration is increased upon local administration of the implantable construct into a body cavity of a subject, i.e., a patient. In an embodiment, the implantable construct comprises a plurality of engineered cells capable of producing a cytokine. In an embodiment, the cytokine is IL-2mt, IL-10, IL-12, IL-23, IL-27, or IL-35 or a functional variant thereof. In an embodiment, the cavity is the intraperitoneal (IP) space. In an embodiment, the diameter of the implantable construct is 1.5 mm. In an embodiment, the engineered cell is an RPE cell, e.g., ARPE-19 cell. In an embodiment, the human c-peptide concentration is between about 200 to about 400 μM. In an embodiment, c-peptide secretion, e.g., human c-peptide secretion, is increased in streptozotocin (STZ)-induced C57BL/6J diabetic mice after administration of the RPE-IL10 implantable constructs in the intraperitoneal (IP) space relative to a control group, e.g., RPE or islet cap groups. In an embodiment, human c-peptide concentration is between about 200 to about 400 μM in streptozotocin (STZ)-induced C57BL/6J diabetic mice after administration of the RPE-IL10 implantable constructs in the intraperitoneal (IP) space.

In an embodiment, local administration of implantable constructs in a subject, i.e., a patient, prevents diabetes onset in 80%, 85%, 90%, 91%, 92, 93%, 94%, 95%, or more after 16 weeks. In an embodiment, local administration of implantable constructs in a diabetes animal model prevents diabetes onset in 80%, 85%, 90%, 91%, 92, 93%, 94%, 95%, or more after 16 weeks. In an embodiment, the implantable construct comprises a plurality of engineered cells capable of producing a cytokine. In an embodiment, the cytokine is IL-2mt, IL-10, IL-12, IL-23, IL-27, or IL-35 or a functional variant thereof. In an embodiment, the cavity is the intraperitoneal (IP) space. In an embodiment, the diameter of the implantable construct is 1.5 mm. In an embodiment, the engineered cell is an RPE cell, e.g., ARPE-19 cell. In an embodiment, implantation of RPE-IL10 implantable constructs in the intraperitoneal (IP) space prevents diabetes onset in 80% or more of NOD mice administered the implantable constructs after 16 weeks.

In an embodiment, about 50% or more of islets in a subject, i.e., a patient, that are locally administered implantable constructs have little or no infiltration by lymphocytes and manifest minimal insulitis, e.g., an insulitis score of 0 or 1. In an embodiment, the implantable construct comprises a plurality of engineered cells capable of producing a cytokine. In an embodiment, the cytokine is IL-2mt, IL-10, IL-12, IL-23, IL-27, or IL-35 or a functional variant thereof. In an embodiment, the cavity is the intraperitoneal (IP) space. In an embodiment, the diameter of the implantable construct is 1.5 mm. In an embodiment, the engineered cell is an RPE cell, e.g., ARPE-19 cell. In an embodiment, about 80% or more of islets in NOD mice that are administered RPE-IL10 implantable constructs implanted in the intraperitoneal (IP) space have little or no infiltration by lymphocytes and have insulitis scores, as assessed by severity, of about 30% with a score of 0/5, about 20% with a score 1/5, and about 30% with score of 2/5.

In an embodiment, a subject manifests no material signs of insulitis or infiltration by lymphocytes upon administration of an implantable construct. In an embodiment, the implantable construct comprises a plurality of engineered cells capable of producing a cytokine. In an embodiment, the cytokine is IL-2mt, IL-10, IL-12, IL-23, IL-27, or IL-35 or a functional variant thereof. In an embodiment, the cavity is the intraperitoneal (IP) space. In an embodiment, the diameter of the implantable construct is 1.5 mm. In an embodiment, the engineered cell is an RPE cell, e.g., ARPE-19 cell. In an embodiment, a diabetic animal model manifests no material signs of insulitis or infiltration by lymphocytes upon administration of an implantable construct comprising a plurality of engineered cells. In an embodiment, islets in diabetic NOD mice that are administered RPE-IL10 implantable constructs implanted in the intraperitoneal (IP) space are not infiltrated by lymphocytes and manifest have an insulitis severity score of 0/5 (i.e., no inflammation). In an embodiment, islets in diabetic NOD mice that are administered RPE-IL10 implantable constructs implanted in the intraperitoneal (IP) space are not infiltrated by lymphocytes and manifest have an insulitis severity score of 1/5. In an embodiment, islets in diabetic NOD mice that are administered RPE-IL10 implantable constructs implanted in the intraperitoneal (IP) space are not infiltrated by lymphocytes and manifest have an insulitis severity score of 2/5.

In an embodiment, cytokine production is detectable as measured by a protein-specific ELISA from a plurality of cytokine-producing engineered cells. In an embodiment, cytokine production is detectable as measured by a protein-specific ELISA from a plurality of cytokine-producing engineered cells encapsulated in an implantable construct. In an embodiment, the number of cells is about 10,000. In an embodiment, the amount of cytokine production is about 200 to about 400 μg/day for 10,000 cells. In an embodiment, the cytokine is IL-35. In an embodiment, the cells are RPE cells, e.g., ARPE-19 cells. In an embodiment, IL-35 production is between about 200 and 400 μg/day for 10,000 free ARPE-19 cells capable of expressing the Flex-IL35 FC plasmid 24 h after transfection as measured by a protein-specific ELISA. In an embodiment, IL-35 production is between about 200 and 400 μg/day for 10,000 ARPE-19 cells capable of expressing the Flex-IL35 FC plasmid encapsulated in alginate implantable constructs 24 h after transfection as measured by a protein-specific ELISA.

In an embodiment, production of a cytokine is detectable as measured with a protein-specific ELISA from the cell supernatant of RPE-IL35 implantable constructs after 24 h. In an embodiment, the cytokine is IL-35. In an embodiment, the cytokine production is about 2000 pg/day. In an embodiment, the cells are RPE cells, e.g., ARPE-19 cells.

In an embodiment, cytokine production is detectable as measured by a protein-specific ELISA from a plurality of cytokine-producing engineered cells. In an embodiment, cytokine production is detectable as measured by a protein-specific ELISA from a plurality of cytokine-producing engineered cells encapsulated in an implantable construct. In an embodiment, the number of cells is about 10,000. In an embodiment, the amount of cytokine production is about 200 to about 300 μg/day for 10,000 cells. In an embodiment, the cytokine is IL12p35 or IL12p35hIL2. In an embodiment, the cells are RPE cells, e.g., ARPE-19 cells. In an embodiment, IL12p35 production is between about 200 to about 300 μg/day for 10,000 ARPE-19 cells capable of expressing the IL12p35hIL2 plasmid encapsulated in alginate implantable constructs 24 h after transfection as measured by a protein-specific ELISA. In an embodiment, IL12p35 production is between about 200 to about 300 μg/day for 10,000 free ARPE-19 cells capable of expressing the IL12p35hIL2 plasmid 24 h after transfection as measured by a protein-specific ELISA.

In an embodiment, cytokine production is detectable as measured by a protein-specific ELISA from a plurality of cytokine-producing engineered cells. In an embodiment, cytokine production is detectable as measured by a protein-specific ELISA from a plurality of cytokine-producing engineered cells encapsulated in an implantable construct. In an embodiment, the number of cells is about 10,000. In an embodiment, the amount of cytokine production is about 15,000 μg/day for 10,000 cells. In an embodiment, the cytokine is IL-23 or IL-27. In an embodiment, the cells are RPE cells, e.g., ARPE-19 cells. In an embodiment, IL-23 production is about 15,000 μg/day for 10,000 free IL-23-expressing ARPE-19 cells as measured by a protein-specific ELISA. In an embodiment, IL-27 production is about 15,000 μg/day for 10,000 free IL-27-expressing ARPE-19 cells encapsulated in alginate implantable constructs 24 h after transfection as measured by a protein-specific ELISA. In an embodiment, IL-23 production is about 15,000 μg/day for 10,000 IL-23-expressing ARPE-19 cells encapsulated in alginate implantable constructs 24 h after transfection as measured by a protein-specific ELISA. In an embodiment, IL-27 production is about 15,000 μg/day for 10,000 IL-27-expressing ARPE-19 cells encapsulated in alginate implantable constructs 24 h after transfection as measured by a protein-specific ELISA.

### Pharmaceutical Compositions

The implantable constructs may be formulated to deliver a therapeutic agent, e.g., systemically or locally. In an embodiment, the implantable constructs are formulated for systemic delivery of a therapeutic agent, e.g., a protein such as a cytokine, by disposing, e.g., implanting, said implantable constructs in the intraperitoneal cavity. In an embodiment, the implantable constructs are formulated for local delivery of a therapeutic agent, e.g., a protein such as a cytokine, by disposing, e.g., implanting, said implantable constructs in the intraperitoneal cavity. The composition featuring the implantable constructs may further comprise a pH modifier, a tonicity agent, a viscosity modifier, a carrier or diluent (e.g., a pharmaceutically acceptable carrier or diluent), a preservative, a surfactant, or a polymer.

In an embodiment, the composition comprises a pH modifier. A function of the pH modifier is to maintain the pH of the composition within a predetermined range, which is optimal for administration of the composition, efficacious for delivery of the therapeutic agent (e.g., the cytokine, the chemokine, secondary polypeptide therapeutic, or a combination thereof), ensures the stability or prevents the degradation or hydrolysis of the therapeutic agent, and the like. Suitable pH modifiers include pharmaceutically acceptable buffering agents, e.g., a combination of a weak Lewis acid and/or its conjugate base. Exemplary pH modifiers featured in the implantable construct include, without limitation, adipic acid, ammonium bicarbonate, potassium citrate monohydrate, sodium hydrogen carbonate, L-tartaric acid, and

The composition features a pH modifier such that the pH of the composition is maintained within a predetermined range. In some embodiments, the pH of the composition is between about 3 to about 10, about 4 to about 9, about 5 to about 8, or about 6 to about 7. In some embodiments, the pH of the composition is between about 3 to about 10. In some embodiments, the pH of the composition is between about 4 to about 9. In some embodiments, the pH of the composition is between about 5 to about 8. In some embodiments, the pH of the implantable construct is between about 6 to about 7.

In some embodiments, the composition featuring the implantable constructs comprises a tonicity agent. Exemplary tonicity agents include dextrose, glycerin, mannitol, potassium chloride, sodium chloride, and the like.

In an embodiment, the composition may comprise between about 0.1% w/w to about 20% w/w of the tonicity agent. In an embodiment, the composition comprises between about 0.1% w/w to about 20% w/w, about 0.2% w/w to about 20% w/w, about 0.3% w/w to about 20% w/w, about 0.4% w/w to about 20% w/w, about 0.5% w/w to about 20% w/w, about 1% w/w to about 20% w/w, about 2% w/w to about 20% w/w, about 3% w/w to about 20% w/w, about 4% w/w to about 20% w/w, about 5% w/w to about 20% w/w, about 7.5% w/w to about 20% w/w, about 10% w/w to about 20% w/w, about 12.5% w/w to about 20% w/w, about 15% w/w to about 20% w/w, or about 17.5% w/w to about 20% w/w of the tonicity agent. In an embodiment, the composition comprises between about 0.2% w/w to about 20% w/w of the tonicity agent. In an embodiment, the composition comprises between about 0.3% w/w to about 20% w/w of the tonicity agent. In an embodiment, the composition comprises between about 0.4% w/w to about 20% w/w of the tonicity agent. In an embodiment, the composition comprises between about 0.5% w/w to about 20% w/w of the tonicity agent. In an embodiment, the composition comprises between about 1% w/w to about 20% w/w of the tonicity agent. In an embodiment, the composition comprises between about 2% w/w to about 20% w/w of the tonicity agent. In an embodiment, the composition comprises between about 3% w/w to about 20% w/w of the tonicity agent. In an embodiment, the composition comprises between about 4% w/w to about 20% w/w of the tonicity agent. In an embodiment, the composition comprises between about 5% w/w to about 20% w/w of the tonicity agent. In an embodiment, the composition comprises between about 7.5% w/w to about 20% w/w of the tonicity agent. In an embodiment, the composition comprises between about 10% w/w to about 20% w/w of the tonicity agent. In an embodiment, the composition comprises between about 12.5% w/w to about 20% w/w of the tonicity agent. In an embodiment, the composition comprises between about 15% w/w to about 20% w/w of the tonicity agent. In an embodiment, the composition comprises between about 17.5% w/w to about 20% w/w of the tonicity agent.

In an embodiment, the composition featuring implantable constructs comprises a viscosity modifier. Suitable pharmaceutically acceptable viscosity modifiers include guar gum, xanthan gum, gellan gum, dextran, pullulan, guar gum, acacia gum, carrageenan, pectin, starch or modified starch derivatives, cellulose, carboxymethylcellulose, chitosan, gelatin, hydroxypropyl methylcellulose, methyl hydroxypropyl cellulose, methyl hydroxyethyl cellulose, hydroxypropyl cellulose, nanocellulose, and the like.

The composition may comprise between about 0.1% w/w to about 20% w/w of the viscosity modifier. In an embodiment, the composition comprises between about 0.1% w/w to about 20% w/w, about 0.2% w/w to about 20% w/w, about 0.3% w/w to about 20% w/w, about 0.4% w/w to about 20% w/w, about 0.5% w/w to about 20% w/w, about 1% w/w to about 20% w/w, about 2% w/w to about 20% w/w, about 3% w/w to about 20% w/w, about 4% w/w to about 20% w/w, about 5% w/w to about 20% w/w, about 7.5% w/w to about 20% w/w, about 10% w/w to about 20% w/w, about 12.5% w/w to about 20% w/w, about 15% w/w to about 20% w/w, or about 17.5% w/w to about 20% w/w of the viscosity modifier. In an embodiment, the composition comprises between about 0.2% w/w to about 20% w/w of the viscosity modifier. In an embodiment, the composition comprises between about 0.3% w/w to about 20% w/w of the viscosity modifier. In an embodiment, the composition comprises between about 0.4% w/w to about 20% w/w of the viscosity modifier. In an embodiment, the composition comprises between about 0.5% w/w to about 20% w/w of the viscosity modifier. In an embodiment, the composition comprises between about 1% w/w to about 20% w/w of the viscosity modifier. In an embodiment, the composition comprises between about 2% w/w to about 20% w/w of the viscosity modifier. In an embodiment, the composition comprises between about 3% w/w to about 20% w/w of the viscosity modifier. In an embodiment, the composition comprises between about 4% w/w to about 20% w/w of the viscosity modifier. In an embodiment, the composition comprises between about 5% w/w to about 20% w/w of the viscosity modifier. In an embodiment, the composition comprises between about 7.5% w/w to about 20% w/w of the viscosity modifier. In an embodiment, the composition comprises between about 10% w/w to about 20% w/w of the viscosity modifier. In an embodiment, the composition comprises between about 12.5% w/w to about 20% w/w of the viscosity modifier. In an embodiment, the composition comprises between about 15% w/w to about 20% w/w of the viscosity modifier. In an embodiment, the composition comprises between about 17.5% w/w to about 20% w/w of the viscosity modifier.

In an embodiment, the composition featuring implantable constructs comprises a carrier or diluent, e.g., a pharmaceutically acceptable carrier or diluent. Exemplary pharmaceutically acceptable carriers or diluents include calcium carbonate, calcium phosphate, lactose (e.g., lactose monohydrate), maltodextrin, D-mannitol, microcrystalline cellulose, native (uncooked starch), pregelatinized starch, sucrose, sorbitol, and sodium chloride.

The composition may comprise between about 0.1% w/w to about 20% w/w of the pharmaceutically acceptable carrier or diluent. In an embodiment, the composition comprises between about 0.1% w/w to about 20% w/w, about 0.2% w/w to about 20% w/w, about 0.3% w/w to about 20% w/w, about 0.4% w/w to about 20% w/w, about 0.5% w/w to about 20% w/w, about 1% w/w to about 20% w/w, about 2% w/w to about 20% w/w, about 3% w/w to about 20% w/w, about 4% w/w to about 20% w/w, about 5% w/w to about 20% w/w, about 7.5% w/w to about 20% w/w, about 10% w/w to about 20% w/w, about 12.5% w/w to about 20% w/w, about 15% w/w to about 20% w/w, or about 17.5% w/w to about 20% w/w of the pharmaceutically acceptable carrier or diluent. In an embodiment, the composition comprises between about 0.2% w/w to about 20% w/w of the pharmaceutically acceptable carrier or diluent. In an embodiment, the composition comprises between about 0.3% w/w to about 20% w/w of the pharmaceutically acceptable carrier or diluent. In an embodiment, the composition comprises between about 0.4% w/w to about 20% w/w of the pharmaceutically acceptable carrier or diluent. In an embodiment, the composition comprises between about 0.5% w/w to about 20% w/w of the pharmaceutically acceptable carrier or diluent. In an embodiment, the composition comprises between about 1% w/w to about 20% w/w of the pharmaceutically acceptable carrier or diluent. In an embodiment, the composition comprises between about 2% w/w to about 20% w/w of the pharmaceutically acceptable carrier or diluent. In an embodiment, the composition comprises between about 3% w/w to about 20% w/w of the pharmaceutically acceptable carrier or diluent. In an embodiment, the composition comprises between about 4% w/w to about 20% w/w of the pharmaceutically acceptable carrier or diluent. In an embodiment, the composition comprises between about 5% w/w to about 20% w/w of the pharmaceutically acceptable carrier or diluent. In an embodiment, the composition comprises between about 7.5% w/w to about 20% w/w of the pharmaceutically acceptable carrier or diluent. In an embodiment, the composition comprises between about 10% w/w to about 20% w/w of the pharmaceutically acceptable carrier or diluent. In an embodiment, the composition comprises between about 12.5% w/w to about 20% w/w of the pharmaceutically acceptable carrier or diluent. In an embodiment, the composition comprises between about 15% w/w to about 20% w/w of the pharmaceutically acceptable carrier or diluent. In an embodiment, the composition comprises between about 17.5% w/w to about 20% w/w of the pharmaceutically acceptable carrier or diluent.

In an embodiment, the composition featuring the implantable constructs comprises a preservative. In an embodiment, the composition comprises a preservative with bactericidal, bacteriostatic, anti-fungal, or anti-protozoal activity. In an embodiment, the preservative is a bactericide. In an embodiment, the preservative is an anti-fungal agent. Exemplary preservatives include benzoic acid, benzyl alcohol, benzalkonium chloride, benzethonium chloride, bronidol, butylatehydroxytoluene (BHT), butyl paraben, chlorobutanol, chlorocresol, meta cresol, methyl paraben, phenyl ethyl alcohol, propyl paraben, phenol, propyl gallate, propylene glycol, sodium benzoate, sodium calcium edetate, sorbic acid, thiomersal, Vitamin C, and Vitamin E

The composition may comprise between about 0.1% w/w to about 20% w/w of the preservative. In an embodiment, the composition comprises between about 0.1% w/w to about 20% w/w, about 0.2% w/w to about 20% w/w, about 0.3% w/w to about 20% w/w, about 0.4% w/w to about 20% w/w, about 0.5% w/w to about 20% w/w, about 1% w/w to about 20% w/w, about 2% w/w to about 20% w/w, about 3% w/w to about 20% w/w, about 4% w/w to about 20% w/w, about 5% w/w to about 20% w/w, about 7.5% w/w to about 20% w/w, about 10% w/w to about 20% w/w, about 12.5% w/w to about 20% w/w, about 15% w/w to about 20% w/w, or about 17.5% w/w to about 20% w/w of the preservative. In an embodiment, the composition comprises between about 0.2% w/w to about 20% w/w of the preservative. In an embodiment, the composition comprises between about 0.3% w/w to about 20% w/w of the preservative. In an embodiment, the composition comprises between about 0.4% w/w to about 20% w/w of the preservative. In an embodiment, the composition comprises between about 0.5% w/w to about 20% w/w of the preservative. In an embodiment, the composition comprises between about 1% w/w to about 20% w/w of the preservative. In an embodiment, the composition comprises between about 2% w/w to about 20% w/w of the preservative. In an embodiment, the composition comprises between about 3% w/w to about 20% w/w of the preservative. In an embodiment, the composition comprises between about 4% w/w to about 20% w/w of the preservative. In an embodiment, the composition comprises between about 5% w/w to about 20% w/w of the preservative. In an embodiment, the composition comprises between about 7.5% w/w to about 20% w/w of the preservative. In an embodiment, the composition comprises between about 10% w/w to about 20% w/w of the preservative. In an embodiment, the composition comprises between about 12.5% w/w to about 20% w/w of the preservative. In some embodiments, the composition comprises between about 15% w/w to about 20% w/w of the preservative. In an embodiment, the composition comprises between about 17.5% w/w to about 20% w/w of the preservative.

In an embodiment, the composition featuring implantable constructs comprises a surfactant. In an embodiment, the surfactant comprises an anionic surfactant. In an embodiment, the anionic surfactant is a (C₁₀-C₂₀) carboxylate salt, e.g., a salt of capric acid, undecylic acid, lauric acid, tridecylic acid, myristic acid, pentadecylic acid, palmitic acid, margaric acid, stearic acid, nonadecylic acid, or arachidinic acid, wherein the counterion is selected from an alkali metal cation, e.g., sodium. In an embodiment, the anionic surfactant is an alkyl (poly)ether sulfate. Exemplary alkyl (poly)ether sulfates include ammonium lauryl sulfate, sodium dodecyl sulfate, sodium lauryl sulfate, and sodium pareth sulfate.

In an embodiment, the surfactant comprises a cationic surfactant. In an embodiment, the cationic surfactant is an alkyl benzalkonium chloride.

In an embodiment, the surfactant comprises a zwitterionic (e.g., an amphoteric) surfactant. In an embodiment, the zwitterionic (e.g., amphoteric) surfactant is a zwitterionic phospholipid, e.g., phosphatidylcholine, e.g., lecithin, e.g., soy lecithin or egg lecithin. In an embodiment, the zwitterionic (e.g., amphoteric) surfactant is cocamidopropyl betaine, or cocamidopropyl hydroxysultaine.

In an embodiment, the surfactant comprises a nonionic surfactant. In an embodiment, the nonionic surfactant is an alcohol ethoxylate, a polyethoxylated glycol ether, a polysorbate, a sorbitan ester, or poly(ethylene oxide)-b-poly(propylene oxide)-b-poly(ethylene oxide) (PEO-PPO-PEO).

In an embodiment, the surfactant is selected from any of the pharmaceutically relevant surfactants disclosed in Tadros, T. F. Applied surfactants: principles and applications. 2006: John Wiley & Sons.

The composition may comprise between about 0.1% w/w to about 20% w/w of the surfactant. In an embodiment, the composition comprises between about 0.1% w/w to about 20% w/w, about 0.2% w/w to about 20% w/w, about 0.3% w/w to about 20% w/w, about 0.4% w/w to about 20% w/w, about 0.5% w/w to about 20% w/w, about 1% w/w to about 20% w/w, about 2% w/w to about 20% w/w, about 3% w/w to about 20% w/w, about 4% w/w to about 20% w/w, about 5% w/w to about 20% w/w, about 7.5% w/w to about 20% w/w, about 10% w/w to about 20% w/w, about 12.5% w/w to about 20% w/w, about 15% w/w to about 20% w/w, or about 17.5% w/w to about 20% w/w of the surfactant. In an embodiment, the composition comprises between about 0.2% w/w to about 20% w/w of the surfactant. In an embodiment, the composition comprises between about 0.3% w/w to about 20% w/w of the surfactant. In an embodiment, the composition comprises between about 0.4% w/w to about 20% w/w of the surfactant. In an embodiment, the composition comprises between about 0.5% w/w to about 20% w/w of the surfactant. In an embodiment, the composition comprises between about 1% w/w to about 20% w/w of the surfactant. In an embodiment, the composition comprises between about 2% w/w to about 20% w/w of the surfactant. In an embodiment, the composition comprises between about 3% w/w to about 20% w/w of the surfactant. In an embodiment, the composition comprises between about 4% w/w to about 20% w/w of the surfactant. In an embodiment, the composition comprises between about 5% w/w to about 20% w/w of the surfactant. In an embodiment, the composition comprises between about 7.5% w/w to about 20% w/w of the surfactant. In an embodiment, the composition comprises between about 10% w/w to about 20% w/w of the surfactant. In an embodiment, the composition comprises between about 12.5% w/w to about 20% w/w of the surfactant. In an embodiment, the composition comprises between about 15% w/w to about 20% w/w of the surfactant. In an embodiment, the composition comprises between about 17.5% w/w to about 20% w/w of the surfactant.

In an embodiment, the composition featuring implantable constructs further comprises a polymer. Exemplary polymers include polyethylene glycol (PEG), polyvinylpyrrolidone (PVP), polylactic acid (PLA), poly(ε-caprolactone) (PCL), and any copolymer thereof.

The composition may comprise between about 0.1% w/w to about 20% w/w of the polymer. In an embodiment, the composition comprises between about 0.1% w/w to about 20% w/w, about 0.2% w/w to about 20% w/w, about 0.3% w/w to about 20% w/w, about 0.4% w/w to about 20% w/w, about 0.5% w/w to about 20% w/w, about 1% w/w to about 20% w/w, about 2% w/w to about 20% w/w, about 3% w/w to about 20% w/w, about 4% w/w to about 20% w/w, about 5% w/w to about 20% w/w, about 7.5% w/w to about 20% w/w, about 10% w/w to about 20% w/w, about 12.5% w/w to about 20% w/w, about 15% w/w to about 20% w/w, or about 17.5% w/w to about 20% w/w of the polymer. In an embodiment, the composition comprises between about 0.2% w/w to about 20% w/w of the polymer. In an embodiment, the composition comprises between about 0.3% w/w to about 20% w/w of the polymer. In an embodiment, the composition comprises between about 0.4% w/w to about 20% w/w of the polymer. In an embodiment, the composition comprises between about 0.5% w/w to about 20% w/w of the polymer. In an embodiment, the composition comprises between about 1% w/w to about 20% w/w of the polymer. In an embodiment, the composition comprises between about 2% w/w to about 20% w/w of the polymer. In an embodiment, the composition comprises between about 3% w/w to about 20% w/w of the polymer. In an embodiment, the composition comprises between about 4% w/w to about 20% w/w of the polymer. In an embodiment, the composition comprises between about 5% w/w to about 20% w/w of the polymer. In an embodiment, the composition comprises between about 7.5% w/w to about 20% w/w of the polymer. In an embodiment, the composition comprises between about 10% w/w to about 20% w/w of the polymer. In an embodiment, the composition comprises between about 12.5% w/w to about 20% w/w of the polymer. In an embodiment, the composition comprises between about 15% w/w to about 20% w/w of the polymer. In an embodiment, the composition comprises between about 17.5% w/w to about 20% w/w of the polymer.

### Delivery Modes

### Local Delivery

The present disclosure provides methods for delivery of the composition featuring implantable constructs and associated dosages, e.g., a predetermined, e.g., a prescribed dosage, or dosing schedule. In some embodiments, the implantable construct may disposed, e.g., implanted into a cellular compartment, e.g., the intraperitoneal cavity, for systemic delivery of a therapeutic agent, e.g., a protein such as a cytokine, at a predetermined dosage.

Dosages. The composition may be provided at a predetermined dosage of the therapeutic agent of between about 10 ng to about 10 mg. In an embodiment, the composition is provided at a dosage of the therapeutic agent of between about 10 ng to about 10 mg, about 20 ng to about 10 mg, about 30 ng to about 10 mg, about 40 ng to about 10 mg, about 50 ng to about 10 mg about 100 ng to about 10 mg, about 200 ng to about 10 mg, 300 ng to about 10 mg, 400 ng to about 10 mg, 500 ng to about 10 mg, 1 μg to about 10 mg, 2 μg to about 10 mg, 3 μg to about 10 mg, 4 μg to about 10 mg, 5 μg to about 10 mg, 10 μg to about 10 mg, 20 μg to about 10 mg, 30 μg to about 10 mg, 40 μg to about 10 mg, 50 μg to about 10 mg, 100 μg to about 10 mg, 200 μg to about 10 mg, 300 μg to about 10 mg, 400 μg to about 10 mg, 500 μg to about 10 mg, 1 mg to about 10 mg, 2 mg to about 10 mg, 3 mg to about 10 mg, 4 mg to about 10 mg, or 5 mg to about 10 mg. In an embodiment, the composition is provided at a dosage of the therapeutic agent of between about 20 ng to about 10 mg. In an embodiment, the composition is provided at a dosage of the therapeutic agent of between about 30 ng to about 10 mg. In an embodiment, the composition is provided at a dosage of the therapeutic agent of between about 40 ng to about 10 mg. In an embodiment, the composition is provided at a dosage of the therapeutic agent of between about 50 ng to about 10 mg. In an embodiment, the composition is provided at a dosage of the therapeutic agent of between about 100 ng to about 10 mg. In an embodiment, the composition is provided at a dosage of the therapeutic agent of between about 200 ng to about 10 mg. In an embodiment, the composition is provided at a dosage of the therapeutic agent of between about 300 ng to about 10 mg. In an embodiment, the composition is provided at a dosage of the therapeutic agent of between about 400 ng to about 10 mg. In an embodiment, the composition is provided at a dosage of the therapeutic agent of between about 500 ng to about 10 mg. In an embodiment, the composition is provided at a dosage of the therapeutic agent of between about 1 μg to about 10 mg. In an embodiment, the composition is provided at a dosage of the therapeutic agent of between about 2 μg to about 10 mg. In an embodiment, the composition is provided at a dosage of the therapeutic agent of between about 3 μg to about 10 mg. In an embodiment, the composition is provided at a dosage of the therapeutic agent of between about 4 μg to about 10 mg. In an embodiment, the composition is provided at a dosage of the therapeutic agent of between about 5 μg to about 10 mg. In an embodiment, the composition is provided at a dosage of the therapeutic agent of between about 10 μg to about 10 mg. In an embodiment, the composition is provided at a dosage of the therapeutic agent of between about 20 μg to about 10 mg. In an embodiment, the composition is provided at a dosage of the therapeutic agent of between about 30 μg to about 10 mg. In an embodiment, the composition is provided at a dosage of the therapeutic agent of between about 40 μg to about 10 mg. In an embodiment, the composition is provided at a dosage of the therapeutic agent of between about 50 μg to about 10 mg. In an embodiment, the composition is provided at a dosage of the therapeutic agent of between about 100 μg to about 10 mg. In an embodiment, the composition is provided at a dosage of the therapeutic agent of between about 200 μg to about 10 mg. In an embodiment, the composition is provided at a dosage of the therapeutic agent of between about 300 μg to about 10 mg. In an embodiment, the composition is provided at a dosage of the therapeutic agent of between about 400 μg to about 10 mg. In an embodiment, the composition is provided at a dosage of the therapeutic agent of between about 500 μg to about 10 mg. In an embodiment, the composition is provided at a dosage of the therapeutic agent of between about 1 mg to about 10 mg. In an embodiment, the composition is provided at a dosage of the therapeutic agent of between about 2 mg to about 10 mg. In an embodiment, the composition is provided at a dosage of the therapeutic agent of between about 3 mg to about 10 mg. In an embodiment, the composition is provided at a dosage of the therapeutic agent of between about 4 mg to about 10 mg. In an embodiment, the composition is provided at a dosage of the therapeutic agent of between about 5 mg to about 10 mg.

Agents. In an embodiment, the therapeutic agent comprises a cytokine, a chemokine, a secondary polypeptide therapeutic, or a combination thereof.

In an embodiment, the engineered cell is capable of secreting a protein, e.g., a cytokine. In an embodiment, cytokine is a cytokine of the IL-12 family, e.g., IL-12, IL-23, IL-27, or IL-35. In an embodiment, the cytokine is IL-12. In an embodiment, the cytokine is IL-23. In an embodiment, the cytokine is IL-27. In an embodiment, the cytokine is IL-35.

Exemplary proinflammatory cytokines include interferon gamma (IFNγ), interleukin-1 alpha (IL-1α), interleukin-1 beta (IL-1β), interleukin-2 (IL-2), interleukin-1 beta (IL-1β), interleukin-6 (IL-6), interleukin-8 (IL-8), interleukin-12 (IL-12), interleukin-17A (IL-17A), interleukin 18 (IL-18), interleukin-23 (IL-23) interleukin-33 (IL-33), interleukin-36 alpha (IL-36a), interleukin-36 beta (IL-36β), interleukin-36 gamma (IL-36γ), tumor necrosis factor alpha (TNFα), and tumor necrosis factor alpha (TNFα).

Exemplary anti-inflammatory cytokines include interferon beta (IFNβ), interleukin-2 (IL-2), interleukin 4 (IL-4), interleukikn-5 (IL-5) interleukin-6 (IL-6), interleukin-10 (IL-10), interleukin-13 (IL-13), interleukin-19 (IL-19), interleukin-27 (IL-27), interleukin-33 (IL-33), interleukin-35 (IL-35), interleukin-37 (IL-36), interleukin-38 (IL-38), and transforming growth factor beta (TGFβ).

Exemplary immunomodulatory cytokines include interferon alpha (IFNα), interleukin-1 receptor antagonist protein (IL-1Ra), interleukin-27 (IL-27), and interleukin-36 receptor antagonist (IL-36Ra)

Exemplary proinflammatory chemokines include chemokine (C—C motif) ligand 2 (CCL2), chemokine (C—C motif) ligand 3, (CCL3), chemokine (C—C motif) ligand 5 (CCL5), chemokine (C—C motif) ligand 20 (CCL20), chemokine (C—X—C motif) ligand 1 (CXCL1), chemokine (C—X—C motif) ligand 4 (CXCL4), chemokine (C—X—C motif) ligand 5 (CXCL5), chemokine (C—X—C motif) ligand 6 (CXCL6), chemokine (C—X—C motif) ligand 7 (CXCL7), chemokine (C—X—C motif) ligand 8 (CXCL8), chemokine (C—X—C motif) ligand 9 (CXCL9), and chemokine (C—X—C motif) ligand 10 (CXCL10).

Exemplary anti-inflammatory chemokines include (C—C motif) ligand 17 (CCL17), chemokine (C—C motif) ligand 18 (CCL18), chemokine (C—C motif) ligand 19 (CCL19), chemokine (C—C motif) ligand 21 (CCL21), chemokine (C—X—C motif) ligand 12 (CXCL12), chemokine (C—X—C motif) ligand 13 (CXCL13), and chemokine (C—X—C motif) ligand 16 (CXCL16).

Schedule. The composition be provided at a predetermined dosing schedule, e.g., to maintain a therapeutic concentration of the therapeutic agent. In an embodiment, the composition is provided between four times per day to once per month. In an embodiment, the composition is provided four times per day, three times per day, twice per day, once per day, once every other day, twice per week, once per week, once every two weeks, once every three weeks, once every four weeks, or once per month. In an embodiment, the composition is provided four times per day. In an embodiment, the composition is provided three times per day. In an embodiment, the composition is provided twice per day. In an embodiment, the composition is provided once per day. In an embodiment, the composition is provided once every other day. In an embodiment, the composition is provided twice per week. In an embodiment, the composition is provided once per week. In an embodiment, the composition is provided once every two weeks. In an embodiment, the composition is provided once every three weeks. In an embodiment, the composition is provided once every four weeks. In an embodiment, the composition is provided once per month. In an embodiment, the composition is provided once every three months. In an embodiment, the composition is provided once every six months. In an embodiment, the composition is provided once per year. In an embodiment, the composition is provided once every two years. In an embodiment, the composition is provided once per year. In an embodiment, the composition is provided once every three years. In an embodiment, the composition is provided once per year. In an embodiment, the composition is provided once every four years. In an embodiment, the composition is provided once per year. In an embodiment, the composition is provided once every five years. In an embodiment, the composition is provided once per year. In an embodiment, the composition is provided once every ten years.

The composition featuring implantable constructs may be provided at a predetermined time of the day. In an embodiment, the composition is provided in the morning, e.g., about 6:00 AM, about 7:00 AM, about 8:00 AM, about 9:00 AM, about 10:00 AM, or about 11:00 AM. In an embodiment, the composition is provided in the afternoon, e.g., about 12:00 PM, about 1:00 PM, about 2:00 PM, about 3:00 PM, about 4:00 PM, or about 5:00 PM. In an embodiment, the composition is provided in the evening, e.g., about 6:00 PM, about 7:00 PM, about 8:00 PM, or about 9:00 PM. In an embodiment, the composition is provided at night, e.g., about 10:00 PM, about 11:00 PM, about 12:00 AM, or later.

### Methods of Treatment

Described herein are implantable constructs capable of delivering, in a regulated fashion, a immunomodulatory agent and, optionally, an additional pharmaceutical agent, and related methods of use thereof. In an embodiment, the implantable constructs are used to treat a disease, e.g., as described herein.

In some embodiments, the disease is an autoimmune disease. In an embodiment the autoimmune disease is Type I diabetes.

In some embodiments, the disease is a proliferative disease. In an embodiment, the proliferative disease is cancer. A cancer may be an epithelial, mesenchymal, or hematological malignancy. A cancer includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). In an embodiment, the cancer is a solid tumor (e.g., carcinoid, carcinoma or sarcoma), a soft tissue tumor (e.g., a heme malignancy), or a metastatic lesion, e.g., a metastatic lesion of any of the cancers disclosed herein. In an embodiment, the cancer is a fibrotic or desmoplastic solid tumor.

Exemplary cancers include carcinoma, lymphoma, blastoma, sarcoma, and leukemia or lymphoid malignancies. In an embodiment, the cancer affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, cancer affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

In an embodiment, the implantable construct is used to treat a neurodegenerative disease, autoimmune disease (e.g., diabetes, multiple sclerosis, lupus, occlusions, capsular contractions), or a liver disease (e.g., hepatitis B infection, hepatitis C infection, cirrhosis, or liver cancer) in a subject. In some embodiments, the disease is diabetes (e.g., type 1 diabetes or type 2 diabetes). In some embodiments, the condition is fibrosis. In some embodiments, the condition is inflammation.

The implantable construct described herein may be used in a method to modulate (e.g., upregulate) the immune response in a subject. For example, upon administration to a subject, the implantable construct (or an antigenic and/or therapeutic agent disposed within) may modulate (e.g., upregulate) the level of a component of the immune system in a subject (e.g., increasing the level or decreasing the level of a component). Exemplary immune system components that may be modulated by a method described herein include T cells (e.g., an invasive T cell, a killer T cell, an effector T cell, a memory T cell, a gamma delta T cell, a helper T cell), B cells, antibodies, or other another component.

The implantable constructs described herein may further comprise an additional pharmaceutical agent, such as an anti-proliferative agent, anti-cancer agent, anti-inflammatory agent, an immunomodulatory agent, or a pain-relieving agent, e.g., for use in combination therapy. The additional pharmaceutical agent may be disposed in or on the implantable construct or may be produced by a cell disposed in or on the implantable construct. In an embodiment, the additional pharmaceutical agent is small molecule, a protein, a peptide, a nucleic acid, an oligosaccharide, or other agent.

In an embodiment, the additional pharmaceutical agent is an anti-cancer agent. In some embodiments, the anti-cancer agent is a small molecule, a kinase inhibitor, an alkylating agent, a vascular disrupting agent, a microtubule targeting agent, a mitotic inhibitor, a topoisomerase inhibitor, an anti-angiogenic agent, or an anti-metabolite. In an embodiment, the anti-cancer agent is a taxane (e.g., paclitaxel, docetaxel, larotaxel or cabazitaxel). In an embodiment, the anti-cancer agent is an anthracycline (e.g., doxorubicin). In some embodiments, the anti-cancer agent is a platinum-based agent (e.g., cisplatin or oxaliplatin). In some embodiments, the anti-cancer agent is a pyrimidine analog (e.g., gemcitabine). In some embodiments, the anti-cancer agent is chosen from camptothecin, irinotecan, rapamycin, FK506, 5-FU, leucovorin, or a combination thereof. In other embodiments, the anti-cancer agent is a protein biologic (e.g., an antibody molecule), or a nucleic acid therapy (e.g., an antisense or inhibitory double stranded RNA molecule).

### Patient Selection

The present disclosure provides methods for treating a disorder, e.g., an autoimmune disorder or cancer, featuring administering a composition comprising an implantable construct for delivery of a therapeutic agent, e.g., a protein such as a cytokine. The subject may include a human. In an embodiment, the subject is a female. In an embodiment, the subject is a male. In an embodiment, the subject is aged 18 years or older. In an embodiment, the subject is less than 18 years of age.

The subject may be selected based on having a proliferative disease. In an embodiment, the proliferative disease is cancer. A cancer may be an epithelial, mesenchymal, or hematological malignancy. A cancer includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). In an embodiment, the cancer is a solid tumor (e.g., carcinoid, carcinoma or sarcoma), a soft tissue tumor (e.g., a heme malignancy), or a metastatic lesion, e.g., a metastatic lesion of any of the cancers disclosed herein. In an embodiment, the cancer is a fibrotic or desmoplastic solid tumor.

The subject may be selected based on having cancer. Exemplary cancers include carcinoma, lymphoma, blastoma, sarcoma, and leukemia or lymphoid malignancies. In an embodiment, the cancer affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, cancer affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The subject may be selected based on having a neurodegenerative disease, autoimmune disease e.g., diabetes, multiple sclerosis, lupus, occlusions, capsular contractions), or a liver disease (e.g., hepatitis B infection, hepatitis C infection, cirrhosis, or liver cancer) in a subject. In some embodiments, the disease is diabetes (e.g., type 1 diabetes or type 2 diabetes). In some embodiments, the condition is fibrosis. In an embodiment, the condition is inflammation.

The subject may be selected based on having one or more symptoms of a proliferative disease. In an embodiment, the proliferative disease is cancer. A cancer may be an epithelial, mesenchymal, or hematological malignancy. A cancer includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). In an embodiment, the cancer is a solid tumor (e.g., carcinoid, carcinoma or sarcoma), a soft tissue tumor (e.g., a heme malignancy), or a metastatic lesion, e.g., a metastatic lesion of any of the cancers disclosed herein. In an embodiment, the cancer is a fibrotic or desmoplastic solid tumor.

The subject may be selected based on having one or more symptoms of cancer. Exemplary cancers include carcinoma, lymphoma, blastoma, sarcoma, and leukemia or lymphoid malignancies. In an embodiment, the cancer affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, cancer affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The subject may be selected based on having one or more symptoms of a neurodegenerative disease, autoimmune disease e.g., diabetes, multiple sclerosis, lupus, occlusions, capsular contractions), or a liver disease (e.g., hepatitis B infection, hepatitis C infection, cirrhosis, or liver cancer) in a subject. In some embodiments, the disease is diabetes (e.g., type 1 diabetes or type 2 diabetes). In some embodiments, the condition is fibrosis. In an embodiment, the condition is inflammation.

The subject may be selected based on having had a proliferative disease. In an embodiment, the proliferative disease is cancer. A cancer may be an epithelial, mesenchymal, or hematological malignancy. A cancer includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). In an embodiment, the cancer is a solid tumor (e.g., carcinoid, carcinoma or sarcoma), a soft tissue tumor (e.g., a heme malignancy), or a metastatic lesion, e.g., a metastatic lesion of any of the cancers disclosed herein. In an embodiment, the cancer is a fibrotic or desmoplastic solid tumor.

The subject may be selected based on having had cancer. Exemplary cancers include carcinoma, lymphoma, blastoma, sarcoma, and leukemia or lymphoid malignancies. In an embodiment, the cancer affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, cancer affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The subject may be selected based on having had a neurodegenerative disease, autoimmune disease e.g., diabetes, multiple sclerosis, lupus, occlusions, capsular contractions), or a liver disease (e.g., hepatitis B infection, hepatitis C infection, cirrhosis, or liver cancer) in a subject. In some embodiments, the disease is diabetes (e.g., type 1 diabetes or type 2 diabetes). In an embodiment, the condition is fibrosis. In an embodiment, the condition is inflammation.

### Combination Therapy

The present disclosure provides methods for treating an autoimmune disorder featuring administering implantable constructs for delivery of a therapeutic agent, which may be concomitantly administered with an additional pharmaceutical agent. In an embodiment, the additional pharmaceutical agent is an immunomodulatory agent, e.g., one or more activators of a costimulatory molecule, an inhibitor of an immune checkpoint molecule, or an anti-inflammatory agent. In an embodiment, the immunomodulatory agent is an inhibitor of an immune checkpoint molecule (e.g., an inhibitor of PD-1, PD-L1, LAG-3, TIM-3 or CTLA4, or any combination thereof). In some embodiments, the immunomodulatory agent is a cancer vaccine.

In some embodiments, the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFR beta. In one embodiment, the inhibitor of an immune checkpoint molecule inhibits PD-1, PD-L1, LAG-3, TIM-3 or CTLA4, or any combination thereof. Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof that binds to PD-1, PD-L1, PD-L2, CTLA4, TIM3, LAG3, VISTA, BTLA, TIGIT, LAIR1, CD73, CD160, 2B4 and/or TGFR beta, or a combination thereof. In some embodiments, the immunomodulatory agent is an anti-inflammatory agent, e.g., an anti-inflammatory agent as described herein. In an embodiment, the anti-inflammatory agent is an agent that blocks, inhibits, or reduces inflammation or signaling from an inflammatory signaling pathway. In an embodiment, the anti-inflammatory agent inhibits or reduces the activity of one or more of any of the following an immune component of the subject. In an embodiment, the anti-inflammatory agent is an IL-1 or IL-1 receptor antagonist, such as anakinra, rilonacept, or canakinumab. In an embodiment, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In an embodiment, the anti-inflammatory agent is a TNF-α antagonist, e.g., an anti-TNF-α antibody, such as infliximab (REMICADE®), golimumab (SIMPONI®), adalimumab (HUMIRA®), certolizumab pegol (CIMZIA®) or etanercept. In one embodiment, the anti-inflammatory agent is a corticosteroid, e.g., as described herein.

In an embodiment, the subject is administered an additional therapy, wherein the therapy is useful in the treatment of a proliferative disease. In an embodiment, the proliferative disease is cancer. A cancer may be an epithelial, mesenchymal, or hematological malignancy. A cancer includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). In an embodiment, the cancer is a solid tumor (e.g., carcinoid, carcinoma or sarcoma), a soft tissue tumor (e.g., a heme malignancy), or a metastatic lesion, e.g., a metastatic lesion of any of the cancers disclosed herein. In an embodiment, the cancer is a fibrotic or desmoplastic solid tumor.

In an embodiment, the subject is administered an additional therapy, wherein the therapy is useful in the treatment of cancer. Exemplary cancers include carcinoma, lymphoma, blastoma, sarcoma, and leukemia or lymphoid malignancies. In an embodiment, the cancer affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, cancer affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

In an embodiment, the subject is administered an additional therapy, wherein the therapy is useful in the treatment of neurodegenerative disease, autoimmune disease e.g., diabetes, multiple sclerosis, lupus, occlusions, capsular contractions), or a liver disease (e.g., hepatitis B infection, hepatitis C infection, cirrhosis, or liver cancer) in a subject. In an embodiment, the disease is diabetes (e.g., type 1 diabetes or type 2 diabetes). In an embodiment, the condition is fibrosis. In an embodiment, the condition is inflammation.

### Methods of Making

The present disclosure provides methods for making implantable constructs comprising a plurality of engineered cells for delivery of a therapeutic agent, e.g., a protein such as a cytokine or antibody, to a subject in need thereof.

In an embodiment, the method of making implantable constructs features a device, e.g., an electrostatic spraying device. In a preferred embodiment, the method of making features a device that is a custom-built, coaxial electrostatic spraying device. The custom-built device comprises a voltage-generator that is fixedly connected to a tip of a blunt tipped or coaxial needle and grounded to a bath comprising reagents for crosslinking or forming a hydrogel from a hydrogel-forming polymer. The co-axial needles contain the polymer capable of forming a hydrogel responsive to contacting with a crosslinker, e.g., a barium or calcium salt, e.g., BaCl₂.

In an embodiment, the method of making the implantable constructs features the following steps: (i) fixedly connecting a voltage generator to a tip of a coaxial needle of a co-axial, electrostatic spraying device, wherein the tip of the coaxial needle is grounded to a 1:4 BaCl₂:mannitol cross-linking bath; (ii) combining a mixture comprising 1.4% w/v SLG20 sodium alginate diluted in 0.9% w/v saline with a cell suspension of engineered cells, e.g., ARPE-19 cells, capable of constitutively or inducibly expressing a therapeutic agent, e.g., a cytokine, a chemokine, a secondary therapeutic agent, or a combination thereof, thereby forming an alginate-cell mixture; (iii) loading the alginate-cell mixture of step (ii) into the interior a plurality of the coaxial needles (e.g., 2 coaxial needles); (iv) contacting the alginate-cell mixture with the BaCl₂:mannitol bath, e.g., via applying pressure, e.g., manually or mechanically, to extrude the alginate-cell mixture from the interior of the plurality of the coaxial needles into the BaCl₂:mannitol bath; and (v) responsive to the contacting, crosslinking the alginate-cell mixture, thereby forming barium alginate hydrogel implantable constructs comprising a plurality of engineered cells.

The fabrication technique provides the ability to modulate the diameter of the implantable constructs to between about 50 μm to about 3000 μm. The fabrication technique further allows for making implantable constructs encapsulating between about 250 to about 800,000 cells.

### EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Experimental Methods

The following are exemplary experimental methods employed in the Examples below. The Examples are not limited by the experimental methods recited herein, but merely serve to enable a person of ordinary skill in the art to carry out embodiments of the disclosure.

### Cell Culture and Transfection

Cell culture and transfection procedures were conducted using materials from Fisher Scientific and Invitrogen, including cell culture media and associated reagents. Expression vectors and helper plasmids were obtained from VectorBuilder. Transfection reagents Lipofectamine 3000 and selection antibiotic (puromycin) were purchased from Invitrogen. The Opti-MEM media for transfection was purchased from Thermo Fisher Scientific. The ARPE-19 cell line, procured from ATCC, underwent regular testing for mycoplasma contamination, yielding negative results. Cells were cultured in Dulbecco's Modified Eagle Medium (DMEM/F-12) supplemented with 10% FBS and 1% antibiotic-antimycotic (AA), with media changes occurring three times weekly. ARPE-19 cells were engineered to express cytokines of interest, following the established transfection protocols known in the art. Briefly, ARPE-19 cells were seeded into six well plate at cell density of 500,000 cells per well. The cells in the plate were left overnight in the incubator and primed with 2 mL of Opti-MEM serum free media for 15 minutes prior transfection. A 1:2 ratio of helper plasmid to plasmid expressing cytokine of interest was used to transfect cells. All cell lines were transformed according to the manufacturer's protocol. After incubation of cells with transfection agents for 4 hours at 37° C., the transfection medium was replaced with fresh culture media and cells were left to incubate overnight. Then, the cells were selected for expression with puromycin for 2 weeks and expanded for quantifying the expression via ELISA.

### Engineered Cell Encapsulation

For engineered cell encapsulation, SLG20 alginate (UP-LVG, NovaMatrix, Norway) was dissolved at 1.4% w/v in 0.8% saline, followed by sterile filtration. Before encapsulation, engineered cells underwent trypsinization and centrifugation at 250 g for 5 minutes. Cell pellets were washed twice with Ca-free Krebs buffer and then resuspended in the alginate solution at a density of 5×10⁶ cells/mL (~10,000 cells per implantable construct). Implantable constructs were produced using a custom-built, two-fluid co-axial electrostatic spraying device (Harvard Apparatus), as described previously in the art. Alginate droplets were ejected from an 18 G co-axial needle (Rame-Hart) into a barium chloride crosslinking solution, forming hydrogel implantable constructs with a diameter of 1.5 mm. The flow rate of both syringe pumps for core and shell solutions was adjusted to 5 to 6 mL/hr. Implantable construct size was controlled by adjusting the voltage to between 5.5 and 6 kV (Gamma High Voltage). The implantable constructs were incubated in the crosslinking solution for 15 minutes, washed with HEPES buffer, and maintained using standard cell culture techniques.

### Human Islet Encapsulation

Human islets (Prodo Labs) were cultured in PIM(S) media from the same source. After centrifugation and washing with Ca-free Krebs buffer, the islets were resuspended in a 1.4% SLG20 solution at a density of 20,000 islet equivalents (IEQ) per 2.7 mL. The islet-containing solution was loaded into a syringe with a 25 G blunt-tipped needle. Following the same fabrication procedure as 1.5 mm constructs, the implantable constructs were adjusted to a size of 0.5 mm by setting the voltage to 10 kV with a flow rate of 200 μL/min. After washing, 400 μL aliquots of implantable constructs with 2,000 IEQ cells were prepared for implantation. Additionally, empty implantable constructs, without cells, were fabricated under the same conditions for use in a fibrosis assay.

### Creation of STZ-Induced Diabetic Model

For in vivo studies, a mix of male and female C57BL/6J mice (Charles River Laboratories) aged 8 to 10 weeks was employed. All animal studies were approved by Rice University's Institutional Animal Care and Use Committee (IACUC). To induce insulin-dependent diabetes, healthy C57BL/6J mice received STZ treatment. STZ solution at a concentration of 7.5 mg/ml (50 mg/kg of STZ) was injected into the intraperitoneal (IP) space for five consecutive days. BG levels and weights of the mice were measured after a one-hour fasting. Only mice with BG levels exceeding 350 mg/dL for two consecutive days were deemed diabetic and selected for islet transplantation.

### Intraperitoneal Surgical Implantation of Implantable Constructs in Mice Models

Immunecompetent C57BL/6J mice (8 weeks old) were weighed, anesthetized with 1 to 4% isoflurane in oxygen on a heating pad, and administered subcutaneous Ethiqa XR based on weight. After shaving and sterilizing their abdomens with betadine and isopropanol a 0.5 to 1 cm midline incision was made through the skin. The peritoneal wall was grasped with forceps, and a 5 mm incision was made along the linea alba. For the fibrosis study (1-, 3-, and 6-month time points), 10 implantable constructs containing engineered cells in a 1.5 mm diameter size were implanted into the IP cavity, along with 0.4 mL of empty implantable constructs (0.5 mm size). For the scRNA-seq study, 10 implantable constructs containing engineered cells and 0.4 mL of empty implantable constructs were implanted into the IP space for 7 days. For the islet study with STZ-induced diabetic mice, portions of empty implantable constructs were replaced with islet implantable constructs (with 2,000 IEQ per mouse). In autoimmune disease models using female NOD mice (NOD/ShiLtJ, Jackson Laboratory), only mice before diabetes onset were included. 10 implantable constructs containing IL-10 secreting cells (1.5 mm size) were implanted into the IP space of NOD mice (8 weeks old) until 24 weeks old (for 16 weeks post-implantation). Control groups included a sham group that underwent IP surgery with 1 mL of sterile saline and an RPE group with the same cell density as experimental implantable constructs but containing non-engineered RPE cells. The incision was sealed with a suture.

### Blood Glucose Monitoring

Blood glucose levels were monitored three times weekly after the transplantation of islet implantable constructs, conducted without fasting. Mice displaying BG levels below 250 mg/dL were categorized as normoglycemic. Monitoring persisted until all mice reverted to a hyperglycemic state, at which juncture they were euthanized, and the implantable constructs were recovered.

### Retrieval of Implantable Constructs

At a designated point in the implantation period, mice from each study were euthanized using cardiac puncture procedures followed by cervical dislocation. An incision along the abdomen skin and peritoneal wall was made using forceps and scissors. IP fluid and implantable constructs within the cavity were collected using 10 mL of sterile PBS. Separation of all implantable constructs from the IP fluid was carried out, followed by several washes with Krebs buffer. The implantable constructs were then prepared for further imaging and promptly snap-frozen for future analysis.

### Implantable Construct Imaging and Cell Viability Assay

The implantable constructs were gently washed with Krebs buffer and transferred to 35 mm Petri dishes for bright- and dark-field imaging using an EVOS microscope. Under 2× magnification, images were acquired and stitched to observe the entire dish. Fluorescent imaging of cells stained with the live/dead assay (Invitrogen, catalog #L3224) was performed to assess encapsulated cell viability in both pre- and post-implant implantable constructs. Five implantable constructs from each group underwent washing with PBS and staining with 2 μM calcein AM and 4 μM EthD-1 in complete media. Following a 30-minute incubation, implantable constructs were imaged using an EVOS microscope with fluorescence filters. Live cells were visualized with a GFP filter in green, while dead cells were observed with a Texas-Red filter in red.

scRNA-seq Analysis

On day 7 post-implantation, animals were euthanized, and cell populations from IP fluids and retrieved implantable constructs were collected to analyze the local effects of the therapeutic. Spleens from each group were also collected to examine systemic immune cell populations. After removing red blood cells, cells were resuspended in 1 mL of media for cell counting. Propidium Iodide was added to stain dead cells following the manufacturer's instructions. Live cells, sorted at 1000 cells/μL density in DMEM containing 10% FBS, underwent next-generation sequencing at Baylor College of Medicine. Throughout sample processing and cell sorting, cells were maintained on ice. The Single Cell 5′ Gene Expression Library was generated using the Chromium NextGEM Single Cell Immune Profiling Solution 5′v2 protocol by 10× Genomics. The libraries were sequenced on an Illumina NovaSeq 6000 flow cell. Transcripts in each cell were counted using the 10× Cell Ranger 5.0.1 pipeline, with genome mapping to the mm10 genome build using STAR v2.7.2a. The scRNA-seq data was visualized using UMAP embedding in Loupe Browser 5.0 to identify immune cell populations At this stage, six cell populations were readily apparent. The resulting clusters were assessed for the expression of common immune cell marker genes and were then classified as specific immune cell types based on their expression profiles. The cell identities of each of the five clusters were resolved using the following markers: CD3E (T cells), PRF1 (NK cells), CD19 (B cells), FN1 (monocytes and macrophages), ITGAX (dendritic cells), and LY6G and HDC (granulocytes). These markers assigned individual cell barcodes to their corresponding immune cell type in the Loupe browser. Cell proportions were calculated between cytokines for each immune cell type to assess changes in the infiltrated immune composition. These markers assigned individual cell barcodes to their corresponding immune cell type in the Loupe browser. Cell proportions were calculated between cytokines for each immune cell type to assess changes in the infiltrated immune composition. The significance of these changes was calculated using Fisher's exact test in R (v3.6.1). Differential expression in monocytes between different treatments was derived from the Loupe browser. TGFβ module score was calculated based on expression levels of RPE-IL12-downregulated genes (Tgfb2, Cdh1, Tgfbr1, Smad7, Mapk3, Tab1, Smad3, Crebbp, Skil) in BIOCARTA_TGFB_PATHWAY using addModule method in Seurat (v3.1 5). Expression levels of representative genes from the BIOCARTA_TGFB_PATHWAY pathway and HALLMARK INFLAMMATORY_RESPONSE were z-scaled and plotted in a heatmap using ComplexHeatmap (v2.8.0).

### Enzyme-Linked Immunosorbent Assay (ELISA)

For preimplant implantable constructss, a single implantable construct was added to a 96-well plate (n=8) after encapsulation in 200 μl of culture media at 37° C. in a 5% CO₂ humidified atmosphere. Implantable construct supernatant was collected after 24 hours. For explant implantable constructs, a single implantable construct from each mouse post-retrieval was added to a 96-well plate (n=6) in 200 μl of media for 24 hours and the plate was kept in the incubator. Implantable construct supernatant was collected from each well and assayed. Implantable construct supernatants were assayed at 10×, 100× and 1000× dilutions depending on the ELISA kit. For the local and systemic concentration of cytokines, IP fluid was assayed at 1× and 10× dilution and blood was assayed at 2× and 10× dilution. ELISAs were obtained commercially for mIL10 (R&D Systems, Catalog #: M1000B), mIL12 (R&D Systems, Catalog #: M1270), mIL2 (R&D Systems, Catalog #: M2000). The assay was run according to the manufacturer's protocols. All the samples were run in duplicates.

### Human C-Peptide Assay

Following manufacturer's protocol, blood from each mouse post-explant was assayed in duplicate without dilution (ALPCO, Catalog #: 80-CPTHU-E01.1). All the standards were also assessed in duplicates.

### Immunofluorescence Staining for Confocal Imaging

Retrieved implantable constructs were washed with Krebs buffer, fixed in 4% paraformaldehyde overnight at 4° C., and permeabilized with 1% Triton X-100. Following blocking with a 1% bovine serum albumin (BSA) solution, samples were incubated with antibody cocktails (diluted at 1:200 Alexa Fluor 488 anti-mouse CD68 Antibody (Cat #137012, BioLegend), 1:200 Anti-mouse α-Smooth Muscle-Cy3 (Cat #C₆₁₉₈, Sigma-Aldrich), and DAPI (Cat #R37606, Invitrogen) in 1% BSA) for 1 hour at room temperature. After washing, samples were transferred for imaging using a Nikon Al-Rsi confocal microscope.

### RT-qPCR Analysis

Total RNA was extracted from 100 μL of retrieved implantable constructs (empty implantable constructs with a 0.5 mm size) using the RNeasy Mini Kit (Cat #74104, Qiagen) The extracted RNA was converted to cDNA for RT-qPCR using the high-capacity cDNA reverse transcription kit Cat #4368814, Applied Biosystems). Real-time qPCR (Bio-Rad) was performed using SYBR Green (Cat #A25742, Applied Biosystems), and reactions were run in triplicates under specified conditions. Data analysis was conducted using the 2^{-ΔΔCT} method, comparing relative RNA levels after normalization to mouse ActB and Empty implantable construct control. The primer details are listed in Table 2.

### Histology and Scoring

Treated and control NOD mice were euthanized at 24-weeks of age. Pancreases were harvested and fixed in 10% formaldehyde for 2 days before being transferred to 70% ethanol. The Baylor Pathology and Histology Core performed further processing, sectioning, and histology. Specifically, samples were paraffin-embedded, excess paraffin was trimmed and samples were sectioned saigittally and stained with hematoxylin and eosin (H&E). Insulitis was evaluated by islet scoring as previously described. Briefly, at least 5 islets per pancreas were analyzed and a score between 0 and 4 was given to each. 0=no insulitis, 1=peri-insulitis, 2=infiltration between 25-50% of the islet, 3=infiltration between 50-75% of the islet and 4-almost complete infiltration. The percentage for each scoring per mouse was calculated, and the average percentage of each score was used as a final indicator of the insulitis level for each group.

### Statistical Analysis

All statistical analyses were conducted with GraphPad Prism 9. One-way or two-way ANOVA with Bonferroni multiple-comparison correction was used to determine p values (**** p<0.0001, *** p<0.0002, ** p<0.002, *p<0.033).

TABLE 2
List of RT-qPCR primers.

| Target | Primers (5′→3′) | SEQ ID NO |
|---|---|---|
| Mouse Alpha | Forward: | 6 |
| Smooth muscle | CGCTTCCGCTGCCCAGAGACT | |
| actin (α-SMA) | Reverse: | 7 |
| | TATAGGTGGTTTCGTGGATGCCCGCT | |
| Mouse Collagen | Forward: | 8 |
| 1a1 (Col1a1) | CATGTTCAGCTTTGTGGACCT | |
| | Reverse: | 9 |
| | GCAGCTGACTTCAGGGATGT | |
| Mouse Collagen | Forward: | 10 |
| 1a2 (Col1a2) | GCAGGTTCACCTACTCTGTCCT | |
| | Reverse: | 11 |
| | CTTGCCCCATTCATTTGTCT | |
| Mouse β-actin | Forward: | 12 |
| (ActB) | GCTTCTTTGCAGCTCCTTCGTT | |
| | Reverse: | 13 |
| | CGGAGCCGTTGTCGACGACC | |

TABLE 3
Composition of immune infiltrate in local versus systemic compartments.
Local: Percentage of immune cells in IP/capsule (%)

| Cell | | | | | | | | | | | | |
|---|---|---|---|---|---|---|---|---|---|---|---|---|
| type | | ther | C | ther | K | ther | | ther | ranulo | thers | ono | ther |
| PE | 4.3 | 5.7 | .8 | 8.2 | .5 | 5.5 | 2.8 | 7.2 | 0.6 | 9.4 | 6.1 | 3.9 |
| ham | 0.8 | 9.2 | .2 | 8.8 | .4 | 7.6 | 0.2 | 9.8 | .8 | 5.2 | 0.6 | 9.4 |
| PE | 4.3 | 5.7 | .8 | 8.2 | .5 | 5.5 | 2.8 | 7.2 | 0.6 | 9.4 | 6.1 | 3.9 |
| L-10 | 6.2 | 3.8 | .5 | 7.5 | .8 | 9.2 | .8 | 2.2 | .6 | 2.4 | 5.1 | 4.9 |
| PE | 4.3 | 5.7 | .8 | 8.2 | .5 | 5.5 | 2.8 | 7.2 | 0.6 | 9.4 | 6.1 | 3.9 |
| L-12 | 1.6 | 8.4 | .1 | 8.9 | | 9 | .2 | 3.8 | .2 | 6.8 | 7.0 | 3.0 |

Systemic: Percentage of immune cells in spleen (%)

| ell | | | | | | | | | | | | |
|---|---|---|---|---|---|---|---|---|---|---|---|---|
| type | | ther | C | ther | K | ther | | ther | ranulo | thers | ono | ther |
| PE | 0.5 | 9.5 | .9 | 8.1 | .1 | 1.9 | 2.4 | 7.6 | 6.1 | 3.9 | 1.1 | 8.9 |
| L-10 | 0.5 | 9.5 | .5 | 8.5 | .9 | 6.1 | 1.5 | 8.5 | 6.2 | 3.8 | .4 | 3.6 |
| PE | 0.5 | 9.5 | .9 | 8.1 | .1 | 1.9 | 2.4 | 7.6 | 6.1 | 3.9 | 1.1 | 8.9 |
| L-12 | 6.5 | 3.5 | .7 | 6.3 | .7 | 8.3 | 1.7 | 8.3 | .6 | 1.4 | 7.8 | 2.2 |

### Example 1. Diabetic Reversal Study Using mIL10 Secreting Cells with Human Islets

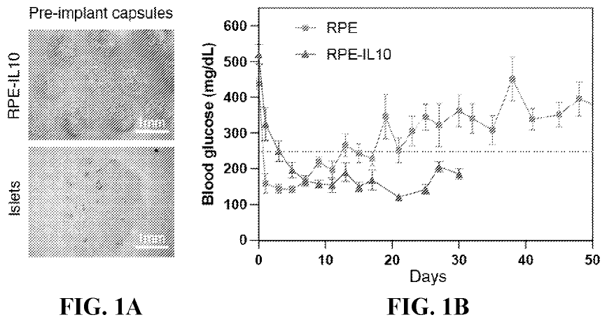

In addition, mIL10-RPE cells were also utilized to correct hyperglycemia of diabetic mice. mIL10-RPE cells were encapsulated within SLG20 alginate at 10,000 cells/implantable construct density with 1.5 mm diameter (FIG. 1A-top image). Non-engineered RPE implantable constructs at the same density were used as a control. 2,000 IEQ of human islets were encapsulated with 0.4 mL of SLG20 alginate (FIG. 1A-bottom image). Islet implantable constructs were fabricated in 0.5 mm diameter to see whether islets can survive with challenging conditions since smaller implantable constructs provoke more immune responses. 10 of 1.5 mm size implantable constructs encapsulating mIL10-RPE cells and 0.4 mL of islet-microimplantable constructs were implanted into STZ-induced diabetic mice. RPE implantable constructs failed to regulate blood glucose levels after 3 weeks, while mIL10-RPE group restored to normoglycemia for the duration of study so far.

### Example 2. Inhibition of Foreign Body Response Utilizing Implantable Construct Comprising Interleukin-Secreting Engineered Cells

The inventors report a new technique for inhibition of the foreign body response to implanted material. The inventors' technology utilizes polymer encapsulated engineered cells that produce natural cytokines such as interleukin-12 (IL-12), interleukin-10 (IL-10), or interleukin-35 (IL-35) to elicit a tailored immune response to implanted materials for applications in diabetes, immune tolerance, and organ transplantation. Further, the inventors report utilization of an inducible kill switch to increase the safety of the inventors' biomaterial platform.

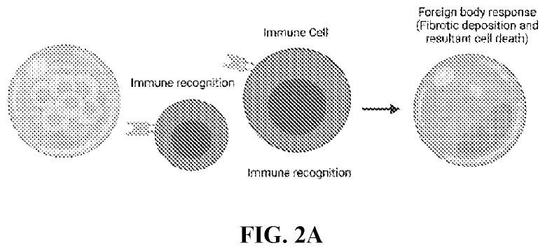

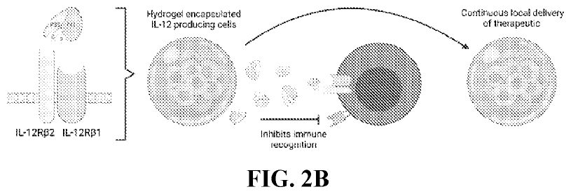

As seen in FIG. 2a, hydrogels are naturally recognized as foreign by immune cells when introduced to the body and subsequently coated with fibrotic deposition as a method of protection for animals. However, the inventors have discovered a system comprised of hydrogel-based capsules that evade activation of this immune response through delivery of heterodimeric IL-12 (FIG. 2b). One subunit, p35, is anti-inflammatory while the other subunit, p40, is pro-inflammatory. Together, these subunits comprise the proinflammatory cytokines IL-12. The inventors have discovered that IL-12 prevents the materials from being coated by the host immune response, thus allowing continuous local delivery of the intended therapeutic (FIG. 2b). This system is customizable by simply spiking IL-12 engineered cells into capsules containing other cytokines of interest. This allows for controlled and predictable modulation of the immune system.

Cells are genetically modified to continuously produce IL-12 using custom designed transfection vectors described here. The heterodimeric protein prevents the implanted hydrogels from being fibrosed and simultaneously causes local immune stimulation and proliferation. Here, the inventors describe a hydrogel-based delivery system composed of cells engineered to make pro-inflammatory cytokines that stimulate and attract immune cells while simultaneously disabling the immune system from mounting an immune response against the implanted materials. Both parts play an important role in coordinating desired responses from the immune system and, when combined, allows for fine-tuned control of immunomodulatory activities.

Cytokines: Cytokines are small molecular weight cell signaling protein that are made by many immune cells in response to stimuli. These proteins function to support, attract and activate/regulate the cells of the immune system. The inventors have designed a cell engineering platform which used synthetic biology principles to create genetically modified cell lines that continuously produce defined concentrations of these proteins inside animals. There are three main classes of cytokines which include pro-inflammatory cytokines which function to activate immune cells, anti-inflammatory cytokines which function to repress immune cells and chemokines which function to initiate immune cell migration. The molecules in these three classes of proteins are largely similar in size and general molecular structure and can thus be each generated with very little very little change in engineering technique. The following cytokines are produced by the inventors' engineered cells: IL-1, IL-1α, IL-1b, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, IL-20, IL-35, IFN-α, IFN-b, IFN-c, TNF-α, TNF-b, TGF-b, CCL-1, CCL-2, CCL-3, CCL-4, CCL-5, CCL-6, CCL-7, CCL-8, CCL-9, CCL-10, CCL-11, CCL-12, CCL-13, CCL-14, CCL-15, CCL-16, CCL-17, CCL-18, CCL-19, CCL-20, CCL-21, CCL-22, CCL-23, CCL-24, CCL-25, CCL-26, CCL-27, CCL-28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17. Control over which cytokines are produced by the inventors' engineered cells allows for precise coordination of the immune system.

Cell engineering for cells equipped with a small molecule inducible kill switch consist of a base epithelial cell line and a highly modular PiggyBAC transposon system. This vector system is composed of a strong promotor that is constitutively expressed, a kozak translation initiation sequence, the gene of interest (any cytokine, chemokine, or other protein), an internal ribosome entry site, a kill switch gene, a puromycin resistance gene and a poly-A tail in between two inverted terminal repeat sequences. Variation to this system requires only gene of interest swapping and allows for quick and easy “plug and play” cell engineering. The kill switch can be added to enhance the safety of cells engineered to produce the following cytokines: IL-1, IL-1a, IL-1b, IL-1RA, IL-2, IL-4, IL-5, IL-6, IL-7, IL-8, IL-9, IL-10, IL-11, IL-12, IL-12a, IL-12b, IL-13, IL-14, IL-15, IL-16, IL-17, IL-20, IFN-α, IFN-b, IFN-c, TNF-α, TNF-b, TGF-b, CCL-1, CCL-2, CCL-3, CCL-4, CCL-5, CCL-6, CCL-7, CCL-8, CCL-9, CCL-10, CCL-11, CCL-12, CCL-13, CCL-14, CCL-15, CCL-16, CCL-18, CCL-19, CCL-20, CCL-21, CCL-22, CCL-23, CCL-24, CCL-25, CCL-26, CCL-27, CCL-28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17. To ensure future clinical translatability, the mouse and human version of cell lines used in this project were created using the appropriate gene sequences from the NCBI. Variation to this system requires only gene of interest swapping and allows for quick and east “plug and play” cell engineering.

Choice of base cell lines includes Chinese hamster ovary (CHO) cells, retinal, pigment epithelial (ARPE-19), human mammary epithelial (MCF-10a and MCF-7), Human embryonic kidney (HEK), mesenchymal stem cells (MSC), Human umbilical vein endothelial cells (HUVEC), NIH/3T3 cells, BJ fibroblasts, Human renal mix epithelial cells (HREC).

Capsule fabrication for mice implant: Modified alginates were initially dissolved at 3-5% w/v in 0.8% saline and then blended with 3% w/v SLG100 (also dissolved in 0.8% saline) at a volume ratio of 70% modified alginate to 30% SLG100. Alginate solutions were sterilized by filtration thorough a 0.2-μm filter. Immediately before encapsulation, the cultured cells were centrifuged at 250G for 5 minutes and washed with Ca-free Krebs-buffer (4.7 mM KCl, 25 mM HEPES, 1.2 mM KH2PO4, 1.2 mM MgSO4·H2O, 135 mM NaCl). After washing, cells were centrifuged again, and all supernatants were aspirated. The cell pellet was then resuspended in alginate solution at the cell density of 1~5×10⁶ cells per 0.5 mL alginate solution. Each HUVECs donor was encapsulated with their corresponding modified alginate solutions. Alginate capsules were made using an electro-spraying machine (Pump 11 Pico Plus, Harvard Apparatus, MA, USA). 18G blunt-tipped needle was attached to a 1-mL Luer-lock syringe containing the alginate solution, clipped to a syringe pump oriented vertically over a 150 mL of crosslinking bath (20 mM BaCl2, 250mMD-Mannitol, 25 mM HEPES with 0.01 v/v % tween 20). A voltage generator was attached to the needle tip and grounded to the crosslinking bath. The settings of the syringe pump were 5 mL/hr flow rate at 15~20 cm height. Cell density per capsule was maintained by adjusting a voltage between 5.5 and 7 kV. After the capsules were formed in the crosslinking bath, they were then collected and washed three times with HEPES buffer (25 mM HEPES (Gibco, Life Technologies, California, USA), 1.2. mM MgCl2×6H2O, 4.7 mM KCl, 1132 mM NaCl). Capsules were washed three times with cell culture medium and cultured overnight in a 37° C. incubator for transplantation.

Capsules can also be made in micrometer size. Modified alginates were dissolved at 3~5% w/v in 0.8% saline and blended with 3% w/v SLG 100 at 70:30 ratio. SLG20 were dissolved at 1.4% w/v in 0.8 saline. Formulated alginate solutions were used to make 300~400 μm size capsules. Encapsulation procedures were the same with 1.5 mm size capsules, except that a 30G needle was used for microcapsules with a 200 μL/min flow rate.

### Generic Strategy for the Synthesis and Characterization of Alginate

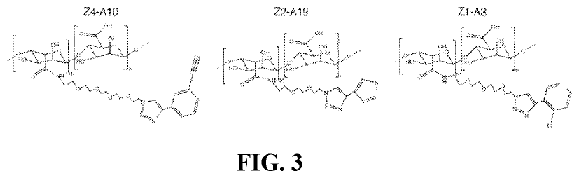

Amide coupling reactions were performed using one equivalent of UP-VLVG alginate and one equivalent of amine linkers (5 different amine linkers) in the presence of a coupling agent of 0.5 equivalent of 4-(4,6-Dimethoxy-1,3,5-triazin-2-yl)-4methylmorpholinium chloride resulting in 5 distinct amine linker conjugated alginate polymers. These linker-modified alginate derivatives were purified by dialysis for three days (in saline and water) using a 10-12 kDa dialysis membrane followed by lyophilization. These five molecules were characterized by NMR and elemental analysis for their purity and % modification of the starting alginates. In the following step, one equivalent of alkynes was conjugated with the appropriately modified alginates by copper-catalyzed click reactions. Triazole-containing alginate derivatives were generated from the synthesis. Below, FIG. 3 is the representative chemical structures of modified alginate formulations.

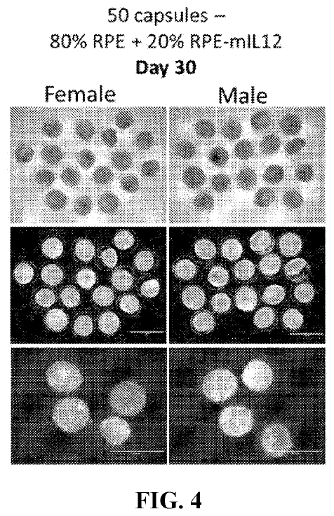

Variation of the implantable cytokine factories will consist of at least 80% cell of interest and 20% RPE-mIL12 cells. These capsules will remain functional at the desired implant site for at least 30 days without becoming fibrosed by the cells of the host immune system. Mixing cells does not affect cell viability as shown in FIG. 4. The cell of interest will be any of the cells listed above, genetically modified to express any of the cytokines listed above. This will allow for optimal modularity of the system for treatment of a wide range of disease implications.

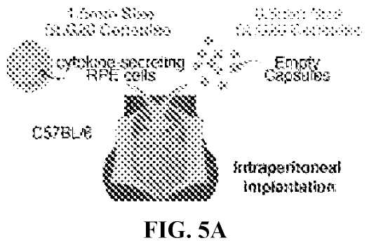

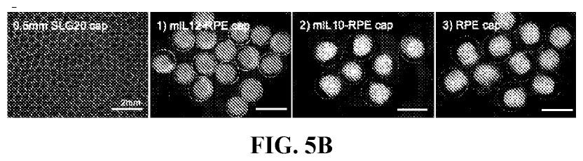

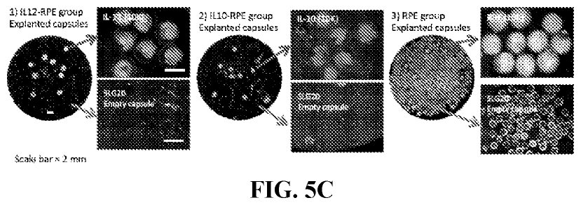

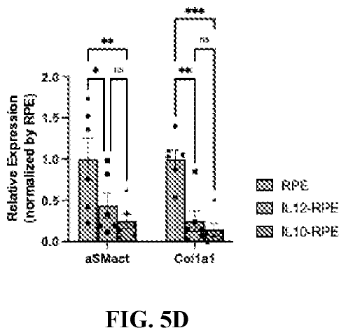

mIL12- and mIL10-producing RPE cells were encapsulated with 1.5 mm alginate capsules. Since smaller sized microcapsules have been reported to produce rapid immune responses, the inventors fabricated empty alginate microcapsules (0.5 mm diameter) to co-deliver with the 1.5 mm capsules encapsulated cells (FIG. 5A). Non-engineered RPE cells were used as control. Each cell line was encapsulated at a density of 10,000 cells per capsule (FIG. 5B), and a total of 10 capsules of either mIL12 or mIL10 capsules were implanted in the IP space together with 0.4 mL of microcapsules. After 4 weeks, all capsules were retrieved and observed under a microscope to visualize fibrosis on the capsule surface (FIG. 5C) Both mIL12 (FIG. 5C1) and mIL10 (FIG. 5C2) groups showed minimal fibrosis deposition onto microcapsules, while the RPE group (FIG. 5C3), had highly packed fibrosis in both large RPE capsules and empty microcapsules. Finally, these groups had lower expressions of fibrotic markers (αSMA and Col1a1) form RT-qPCR analysis (FIG. 5D). This result showed the antifibrotic effect of mIL12- and mIL10-producing cells upon implantation in healthy mice.

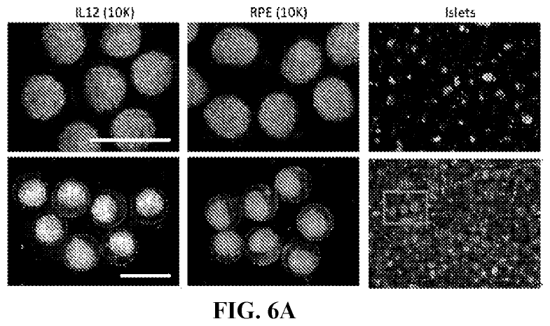

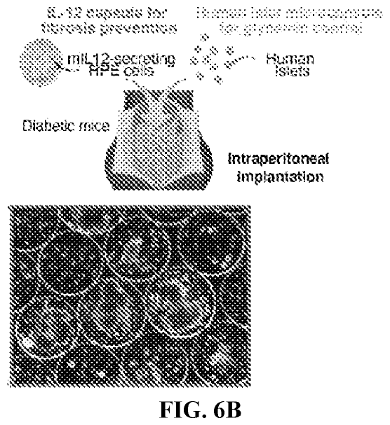

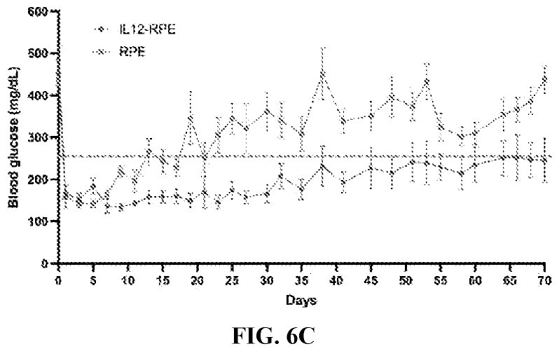

The inventors have confirmed localized cytokine production from encapsulated cells can prevent fibrosis of implants in healthy mice. So, we utilized this approach to co-deliver human islets in diabetic mice. mIL12-RPE cells were encapsulated with SLG20 alginate at 10,000 cells/capsule density with 1.5 mm diameter (FIG. 6A). Non-engineered RPE capsules at the same density were used as a control. 2,000 IEQ of human islets were encapsulated with 0.4 mL of SLG20 (FIG. 6A). Islet capsules were fabricated in 0.5 mm diameter 10 1.5 mm size capsules encapsulating mIL12-RPE cells and 0.4 mL of islet-microcapsules were implanted into STZ-induced diabetic mice (FIG. 6B). RPE capsules failed to regulate blood glucose levels after 3 weeks, while mIL12-RPE group restored to normoglycemia (FIG. 6C).

### Modified Alginate to Deliver Therapeutic Cells

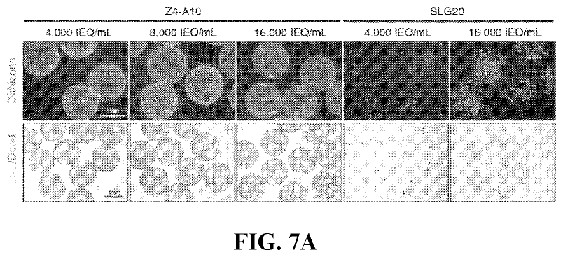

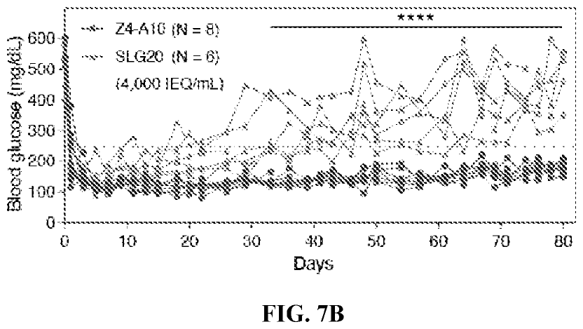

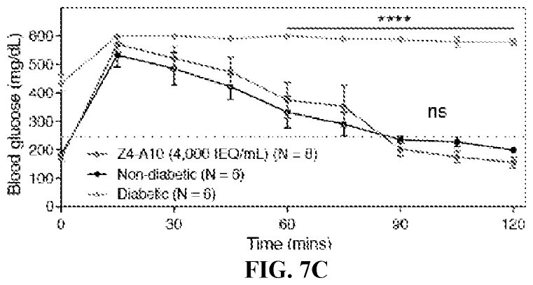

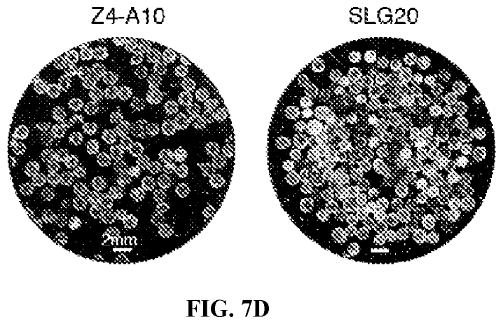

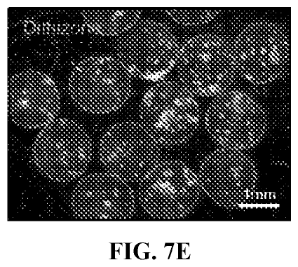

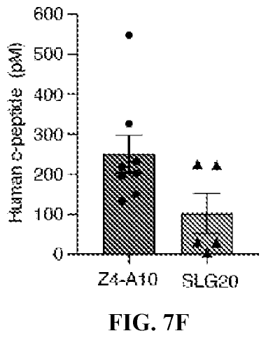

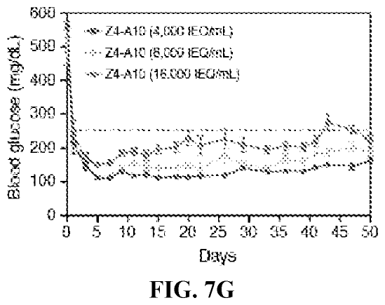

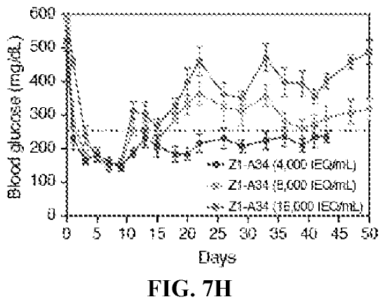

Our newly discovered anti-fibrotic alginate (Z4-A10) hydrogels were used to encapsulate xenogeneic human islets. The formulation provides a highly porous and anti-fibrotic hydrogel outer membrane to enable long-term nutrient diffusion, high islet viability, and low fibrosis in vivo. Capsules with three different densities (4K IEQ/mL of alginate, 8K IEQ/mL of alginate, and 16K IEQ/mL of alginates) of human islets were prepared using Z4-A10 alginates. Previous lead Z1-A34 and control SLG20 capsules were prepared for comparison. The pre-implant dithizone staining and live/dead imaging of the capsule groups (Z4-A10 and control SLG20) demonstrate the viability of the islets (FIG. 7a). The Z4-A10 capsules at a density of 4K IEQ/mL demonstrate long-term restoration of euglycemia and maintain glycemic correction until 80 days of data recording with the average blood glucose (BG) levels below 250, considered as the BG level of a healthy mouse, at a fasting condition (FIG. 7b). However, the control SLG20 alginate at the same dose (IEQ density of 4K/mL) failed to maintain glycemic correction for more than four weeks. Intravenous glucose tolerance test (IVGTT) was performed after four hours of fasting on day 75, showing encapsulated islets cells restored normoglycemia to a rate comparable with healthy C57BL/6J mice (FIG. 7c). Further, the post-retrieval capsule images (dark-field) display minimal fibrotic overgrowth on the surface of Z4-A10 capsules compared with SLG20 (FIG. 7d). Dithizone staining also supports the long-term islet viability after 80 days of implantation (FIG. 7e). The concentration of human c-peptide, a surrogate biomarker for insulin production, was measured from the serum separated from the mouse blood 80 days post-transplantation. Higher levels of c-peptide secretion were observed in the Z4-A10 group compared to SLG20, suggesting better improved long-term viability (FIG. 7f).

In high-density encapsulation groups, Z4-A10 capsules with 8K and 16K IEQ/mL concentration could maintain long-term glycemic control >50 days of function (FIG. 7g). In contrast, the previous lead Z1-A34 and control SLG20 group failed to maintain glycemic control for no more than 10 days of implantation (FIG. 7h-i). However, the previous lead (Z1-A34, FIG. 7h) failed early with higher density groups (at 8K and 16K IEQ/mL), even though it maintained glucose control for 50 days with the lowest density group. The inventors' results suggest that Z4-A10 is enhanced in protecting islets from foreign body response, which maintains longer viability and function of grafts.

The inventors have demonstrated that the lead hydrogel from the inventors' hydrogel screen enables diabetic reversal in immunocompetent C57BL/6J mice when used to encapsulate human islets. FIG. 7a shows representative images of pre-implant capsules. Z4-A10 capsules containing human islets at a density of 10 IEQ/capsule, 20 IEQ/capsule, and 40 IEQ/capsule, respectively. Z1-A34 and SLG20 capsule was used as control material. Dithizone staining indicates viable islets within the capsule matrix. After encapsulation, islets show good viability (live: green, dead: red). FIG. 7b shows blood glucose levels for both Z4-A10 and SLG20 groups (4,000 IEQ/mL density) were monitored until mice were euthanized (**** P<0.0001 (SLG20 vs. Z4-A10)). FIG. 7c shows an IVGTT test with Z4-A10 capsule (4,000 IEQ/mL) implant group in diabetic mouse, an non-implant group in diabetic mice and non-diabetic mice (ns; not significant, **** P<0.0001 (all comparisons)). FIGS. 7d-e show representative dark-field (FIG. 7d), and dithizone staining (red, FIG. 7e) images of explanted Z4-A10 and SLG20 capsules (4,000 IEQ/mL). FIG. 7f shows human c-peptide measurements at 80-days post-transplantation (SLG20 vs. Z4-A10). FIGS. 7g-i show blood glucose monitoring with high islets density groups: Z4-A10 capsules (FIG. 7g), Z1-A34 capsules (FIG. 7h), and SLG capsules (FIG. 7i). Error bars denote mean±sem; Two-way ANOVA with Bonferroni multiple comparison correction.

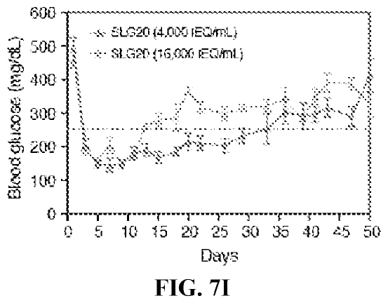

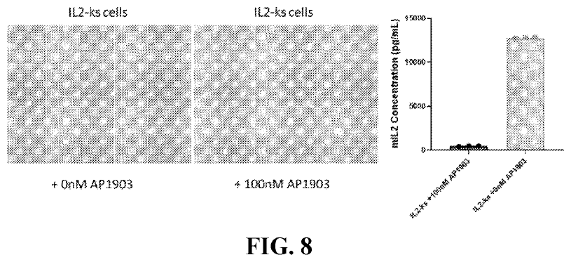

The cytokine producing, kill switch inducible cell line responds to 100 nM AP1903 in less than 24 hours as shown in the images seen in FIG. 8 (left and middle). The cells which were not subjected to the small molecule kill switch inducer remained highly viable, thus allowing for sustained production of the IL-2 protein as shown in FIG. 8 (right). This will allow for increased safety for all future iterations of the inventors' technology.

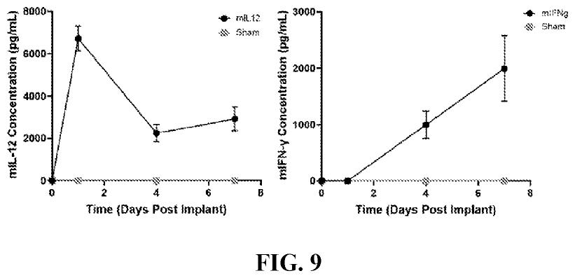

IFN-g can also be used as a reporter for toxicity. Following an increase in the local mIL12 cytokine levels, we saw an increase in local IFN-g concentration that can be utilized as a marker for regulating the production of cytokines from the biomaterial system. Concentration of A) mIL-12 and B) mIFN-γ in the IP-fluid 1-, 4-, and 7-days after RPE-mIL12 administration or sham control. The results are shown in FIG. 9.

### Hydrogel

Refer to patent (RICE.P0068US.P2) about the different types of alginates that will be used. Alginate hydrogel spheres were synthesized using a custom-built, two-fluid co-axial electrostatic spraying device. The device consisted of a voltage generator that was attached to the tip of a co-axial needle and grounded to a 1:4 barium chloride:mannitol crosslinking bath. the co-axial needle was fed by separate syringes containing 1.4% alginate solutions diluted in 0.9% saline.

The modularity of this system will allow for rapid generation and selection of an appropriate delivery system for many diseases. Spatial and temporal control are achieved by varying the ratios of produced cytokines and antigens. For example, capsules filled with different number of cells changes the corresponding dose of cytokine delivered. Capsules can be made with 10,000-80,000 cells per capsules. The ratio of capsules that contain cytokine producing cells and capsules that contain antigen producing cells can also be varied from 1:1 to 100:1 and the therapeutics produced work together to control the immune system. Additionally, a given treatment dose consists of different numbers of capsules for different individuals. For example, dose A consists of 500 capsules at a 1:1 ratio of stimulatory capsules producing chemokines and capsules producing IL-12 to attract immune cells and favor a strong immune cell activation and dose B consists of 1000 capsules at a 50:1 ratio of stimulatory capsules producing cytokines and capsules producing IL-12 to favor a heavier induction of activated immune cells in the local microenvironment.

Capsules range in size from 50 um-3 mm. Some formulations have an additional inner hydrogel for dual layer protection of the encapsulated cells. Capsules of different sizes can hold different concentrations of cells and thus provide an additional means of achieving acute spatial and temporal control over dosing and treatment regimen using the inventors' platform.

Embodiment: Example embodiments for this invention include: 1: diabetes treatment through extended viability of pancreatic islet cells via co-transplantation of encapsulated islet cells and any combination of cytokines to prevent or delay the onset of fibrosis. Continuous local delivery of low dose IL-12, for example, prevents macrophage and other immune cell deposition of pericapsular overgrowth thus allowing long term delivery of the desired treatments. 2: tolerance induction can also be achieved with this system by using low doses of pro- or anti-inflammatory cytokines in combination with IL-12 for an extended time to immune cell migration and B-cell activation. 3: organ transplantation: a small number of IL-12 secreting capsules can be administered along with transplanted organs to prevent initiation of Graft-vs-Host disease and ultimate organ rejection.

### Example 3. Fabrication of Engineered Cytokine-Producing Cells and Encapsulation Thereof

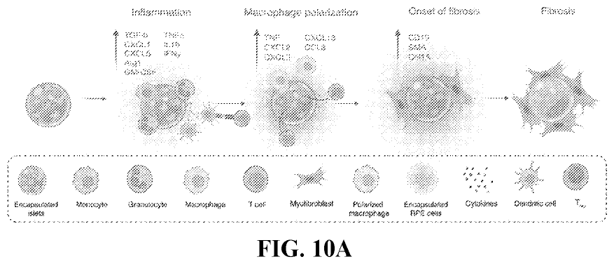

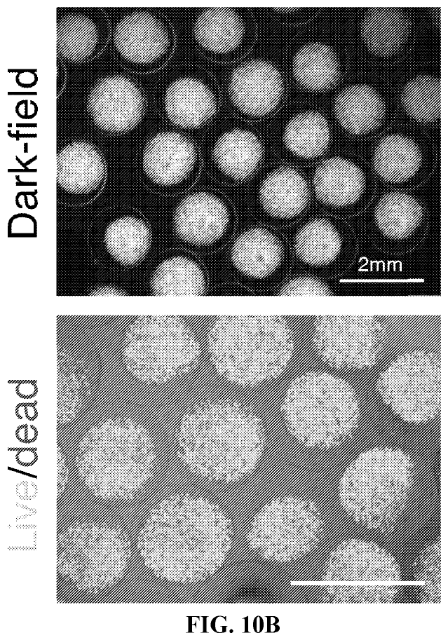

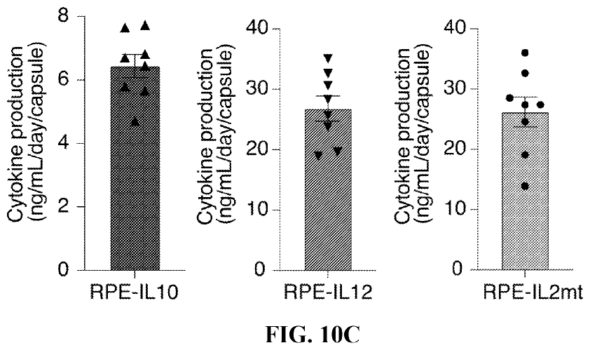

The example set forth below describes the fabrication of engineered cytokine-producing cells and their encapsulation in order to assess their efficacy in the prevention of fibrosis. Briefly, the cytokines IL-10, IL-12, and IL-2mt were first selected to identify the optimal cells lines having the ability to mitigate biomaterial-induced fibrosis. RPE cells were genetically modified to express a specific immunomodulatory cytokine through the PiggyBac transposon system (FIG. 10A). This expression system enables rapid prototype development by substituting the gene of interest while simultaneously preserving the optimized backbone. The aforementioned cytokines were selected due their divergent roles in mediating inflammation: IL-10 is characterized by its potent anti-inflammatory properties and assumes a central role in limiting the host's immune response to pathogens, thereby preventing harm to the host and maintaining normal tissue homeostasis (FIG. 10A); in contrast, IL-12 is characterized as a pro-inflammatory cytokine, which is secreted by dendritic cells and tissue-resident macrophages in response to signals associated with host defense and wound healing (FIG. 10A); IL-2mt possesses reduced affinity for the IL-2Rβγ receptor, resulting in the production of a regulatory T cell (T_{reg})-selective molecule that preferentially expands T_{regS}, while exerting minimal effects on CD4+ and CD8+ memory effector T cells (FIG. 10A). The engineered retinal pigmented epithelial (RPE) cells expressing the selected cytokines of interest were encapsulated in alginate implantable constructs having a diameter of 1.5 mm. Each implantable construct contained approximately 10,000 cells. A LIVE/DEAD assay was subsequently performed, and the cells were determined to be highly viable, as shown in FIG. 10B. Following encapsulation, the cytokine production of implantable constructs prior to implantation was assessed by ELISA after 24 h of incubation, as shown in FIG. 10C. RPE-IL10 implantable constructs (i.e., implantable constructs comprising RPE cells capable of secreting IL-10) exhibited a production rate of approximately 6.4 ng/ml/day/implantable construct, RPE-IL12 yielded about 25.7 ng/ml/day/implantable construct, and RPE-IL2mt produced approximately 26.2 ng/ml/day/implantable construct. The cytokine-producing cell platforms will allow for the production of cytokines continuously and replenish any cytokines which may have degraded over time.

### Example 4. Local Delivery of Anti-Inflammatory and Pro-Inflammatory Cytokines Prevents Fibrosis on the Surface of Biomaterials

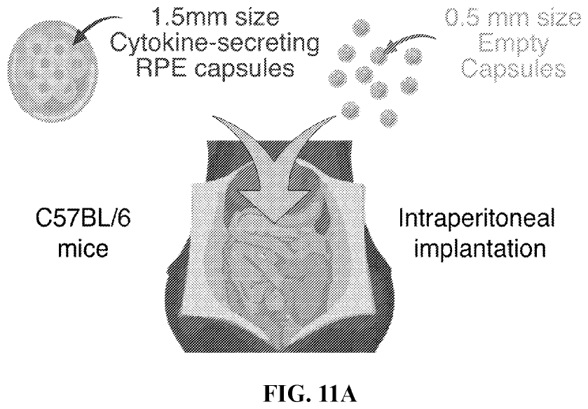

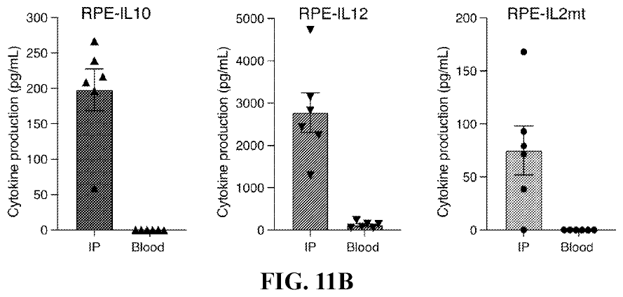

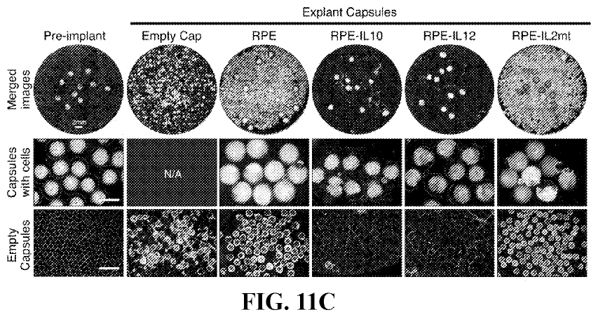

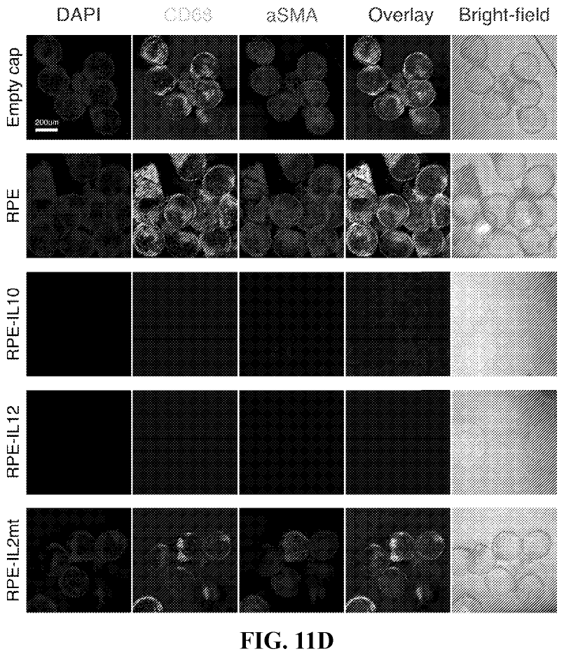

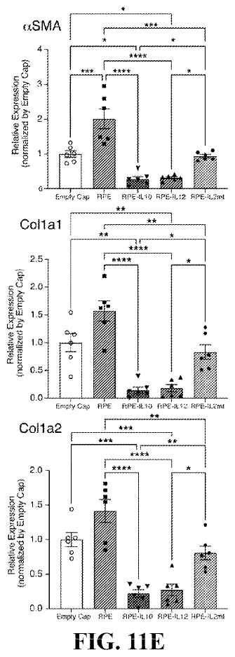

The example set forth below describes the local delivery of anti-inflammatory and pro-inflammatory cytokines for preventing fibrosis on the surface of exemplary biomaterials. Briefly, retinal pigmented epithelial (RPE) cells capable of expressing IL-10, IL-12, IL-2mt, and TGFβ (RPE-IL10, RPE-IL12, RPE-IL2mt, and RPE-TGFβ respectively) were encapsulated in 1.5 mm-diameter alginate implantable constructs. Empty SLG20 alginate microparticles were also fabricated at a 0.5 mm-diameter for co-delivery of encapsulated, cytokine producing cells. The 0.5 mm-diameter was selected in order to induce acute immune responses upon implantation while still being large enough to encapsulate islet cells for a future diabetes study, as shown in FIG. 11A. Non-engineered RPE cells were designated as the control group (i.e., implantable constructs comprising nonengineered RPE cells). Each cell line was encapsulated at a cell density of 10,000 cells per 1.5 mm implantable construct. Large implantable constructs were deliberately chosen so as to create space for delivery of therapeutic agents. 10 implantable constructs were implanted per rodent specimen, thereby delivering about 100,000 cells per mouse. The remaining space in the IP cavity was allocated to empty implantable constructs or implantable constructs comprising human islets for a subsequent diabetic study. The empty implantable constructs served as an additional Control group to compare the rodent immune responses to microimplantable constructs in the absence of xenogeneic cells.

At one month, the production of the selected cytokines at local and systemic levels was assessed as shown in FIG. 11B. Minimal levels of each of the cytokines were measured in blood plasma for all of the cytokine-producing implantable construct groups. Notably, RPE-IL10 and RPE-IL12 exhibited high local concentrations in intraperitoneal (IP) fluid relative to RPE-IL2mt, indicating sustained production of IL-10 and IL-12 in the IP space one month after implantation. Subsequent to the determination of the local and systemic cytokine concentrations, the implantable constructs were retrieved, and fibrosis on their surfaces was observed with optical microscopy (FIGS. 11C & 12). The experimental groups containing non-engineered RPE cells RPE-IL2mt, and RPE-TGFβ manifested dense fibrosis on the implantable construct surfaces recovered from all of the mice, as evidenced by the dark-field microscopy images. In contrast, the RPE-IL10, RPE-IL12, and empty implantable construct groups displayed minimal fibrosis on the implantable construct surfaces. Interestingly, the empty implantable construct group containing no RPE cells still exhibited some fibrotic responses, which is consistent with previous reports in the literature that small implantable construct diameter may promote more robust immune responses. The evaluation of fibrotic markers was subsequently evaluated employing immunofluorescence imaging as shown in FIG. 11D. Retrieved implantable constructs were stained with alpha-smooth muscle actin (aSMA), a myofibroblast marker, and CD68, a macrophage marker. Consistent with dark-field imaging results, expression of fibrotic markers is apparent on the implantable construct surface of the empty implantable construct, non-engineered RPE, and RPE-IL2mt groups. Finally, RT-qPCR was conducted to confirm the expression of fibrosis-related genes using cells collected from retrieved implantable constructs as shown in FIG. 11E. The relative expression of αSMA, Col1a1, and Col1a2 was evaluated among the groups, and the results align with the microscopy findings. Therefore, co-delivery of either IL-10 or IL-12 secreting implantable constructs significantly reduced fibrosis on empty implantable constructs relative to the non-engineered RPE group.

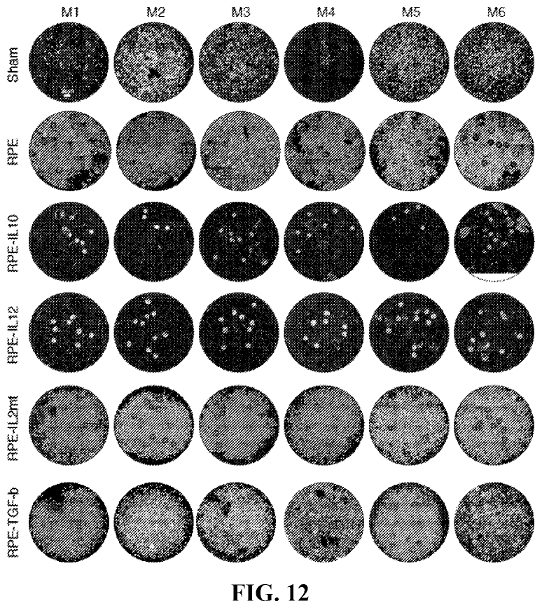

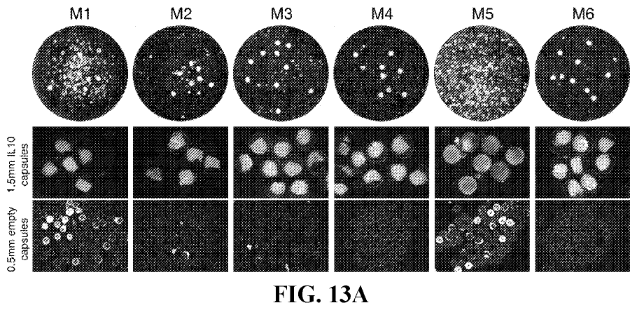

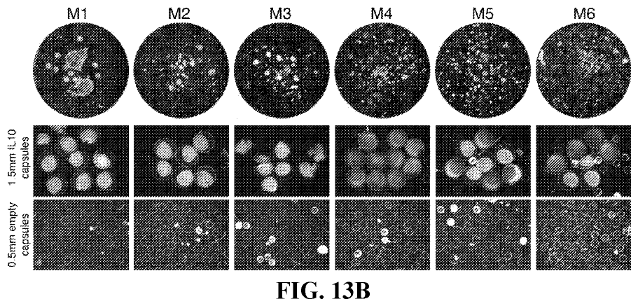

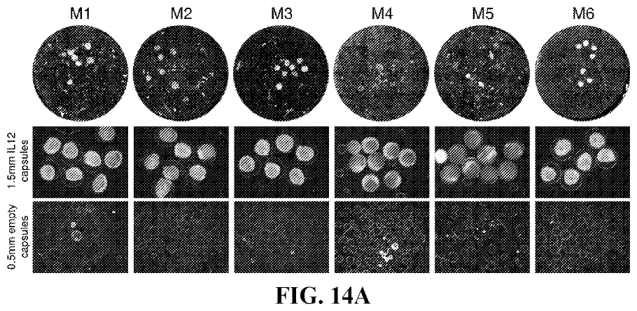

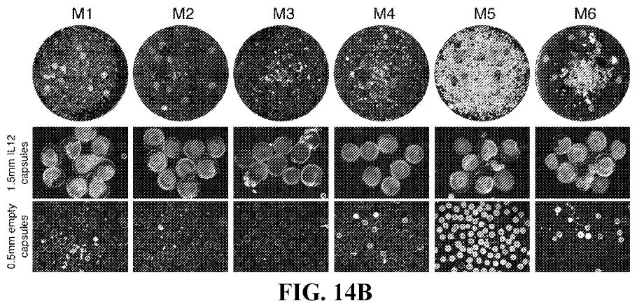

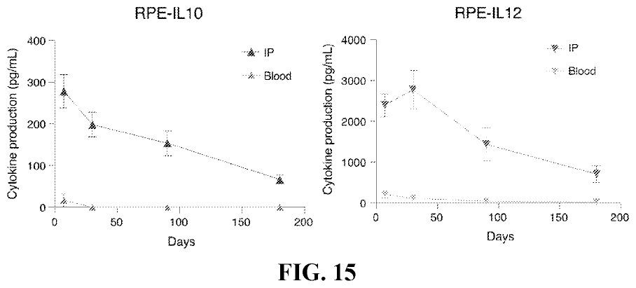

Additionally, the potency of IL-10 and IL-12 secreting cells was observed over an extended period of time. Encapsulated RPE-IL10 or RPE-ILK12 was co-administered into the intraperitoneal space of immunocompetent mice together with empty implantable constructs for durations of three or six months. The RPE-IL10 group demonstrated sustained prevention of fibrosis on the majority of the surfaces of the implantable constructs as shown in FIG. 13. Only a few mice exhibited partial fibrotic deposition on the surface of the RPE-IL10 implantable constructs after three months (FIG. 13A) and six months (FIG. 13B). Similar outcomes were observed in the RPE-IL12 group, indicating robust anti-fibrotic immune modulation, with the exception of a single rodent specimen from the six-month post-implantation group as shown in FIG. 14. To assess potential toxicity of the platform, the local and systemic concentrations of IL-10 and IL-12 were measured over time as shown in FIG. 15. While the concentration of cytokines in the IP space decreased throughout the study period, the encapsulated cytokine-secreting cells continued to produce a measurable amount of cytokines after six months. However, systemic cytokine levels were hardly detectable over the duration of the study and ultimately reverted to being undetectable in the bloodstream, suggesting that there was no systemic cytokine accumulation. Taken together, these findings underscore the potential of locally delivered cytokines to effectively modulate immune response and alleviate fibrosis on the surfaces of biomaterials.

### Example 5. IL-10 Prevents Fibrosis Via Immunosuppression, Whereas IL-12 Prevents Fibrosis by Inhibiting TGFβ in Intraperitoneal Immune Cells

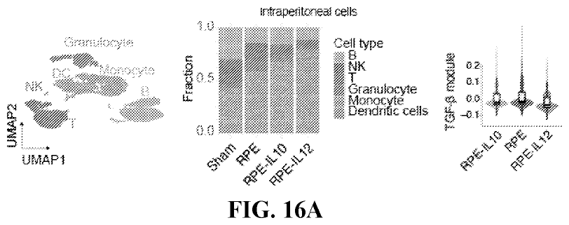

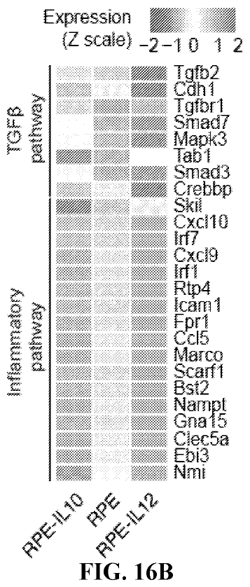

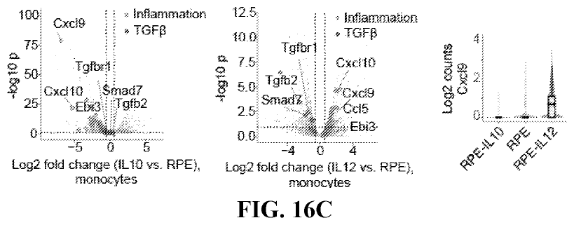

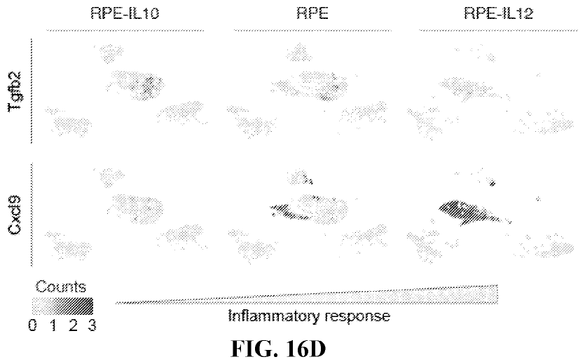

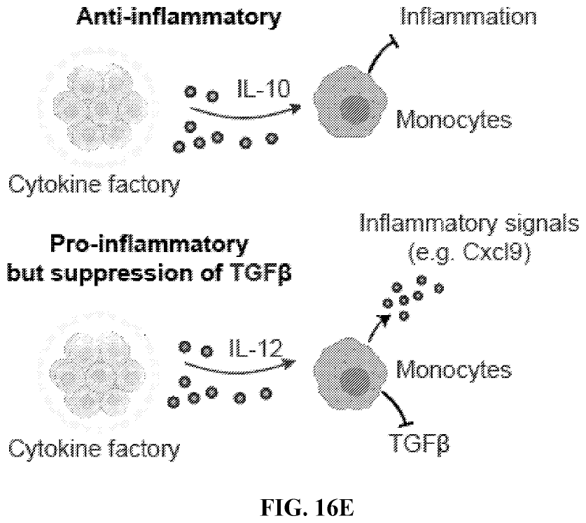

The example set forth below examines the underlying mechanism of fibrosis prevention via RPE-IL10 and RPE-IL12 implantable constructs relative to RPE implantable constructs when foreign materials are introduced. Briefly, cells were collected from either the surface of the implantable construct and surrounding IP fluid or the spleen to evaluate both local and systemic effects. Unform Manifold Approximation and Projection (UMAP) was utilized to identify six distinct cell clusters using standard cell-type specific markers, corresponding to granulocytes, monocytes (e.g., macrophages), dendritic cells, B cells, T cells and natural killer (NK) cells as shown in FIG. 16A.

The experimental groups were organized as follows: Sham refers to sham surgery with saline and no implantable constructs. RPE refers to implantable constructs with encapsulate nonengineered cells that no do not express or secrete cytokines. RPE-IL10 refers to implantable constructs with RPE cells that express IL-10. RPE-IL12 refers to implantable constructs with RPE cells that express IL-12. Subsequent to implantation, implantable constructs were retrieved and cells were collected from either intraperitoneal space or spleen to interrogate for local and systemic effects. The composition of the immune infiltrate was determined to be significantly altered after implantable construct implantation. Single-cell RNA (scRNA) sequence analysis revealed that the composition of immune infiltrate in local space was significantly altered by the cytokine secreting implantable constructs (FIG. 16A). Compared to control RPEs, RPE-IL10 increased the intraperitoneal monocytes by 8.95% (p=1.50e-12), while RPE-IL12 increased the intraperitoneal monocytes by 20.89% (p=1.48e-64). Notably, both cytokines significantly reduced the proportion of granulocytes, T cells, and NK cells by at least 3% (p<1.1e-10 for all) and mildly altered (increased or decreased) the B cells and dendritic cell proportion by less than 3% (p<0.05 for all).

Both anti-fibrotic RPE-IL10 and RPE-IL12 surprisingly enriched the macrophage-containing monocyte population To investigate the effect of RPE-IL10 and RPE-IL12 on fibrotic pathways including inflammatory response and TGFβ pathway in monocytes, the differential expression profiles of monocytes treated with each cytokine was assessed relative to RPE alone. RPE-IL10 significantly suppressed gene sets related to inflammatory response, including allograft rejection (Normalized enrichment score (NES)=−1.74, p=2.8e-4), and antigen processing cross-presentation (NES=−1.74, p=2.1e-3), including the downregulation of inflammatory cytokines Cxc19 expression in monocytes (p<2.2e-16) (FIG. 16A-B, FIG. 14). RPE-IL10 only had mild but not statistically significant effects on fibrotic TGFβ pathway (Normalized enrichment score (NES)=−0.97, p=0.49, FIG. 16A-C, FIG. 14). In contrast, although RPE-IL12 significantly promoted inflammatory response; allograft rejection (NES=1.32, p=0.004) and antigen processing cross-presentation (NES=1.72, p=4.0e-6, FIG. 16A, FIG. 16C, FIG. 15), it significantly inhibited TGFβ pathway (NES=−2.1, p=1.0e-3, FIG. 16A-C, FIG. 15), including downregulation of profibrotic cytokines Tgfb243 (p=4.9e-8, FIG. 16A, FIG. 16C). These results demonstrate that RPE-IL10 and RPE-IL12 exert distinct antifibrotic mechanisms; RPE-IL10 suppresses inflammation, while RPE-IL12 inhibits the TGFβ pathway.

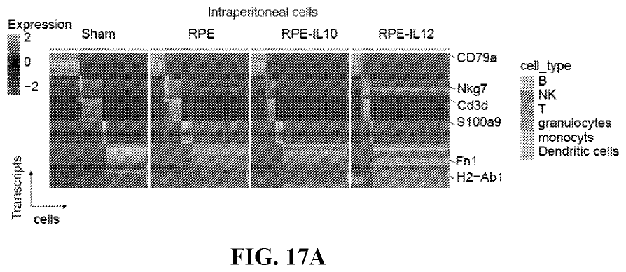

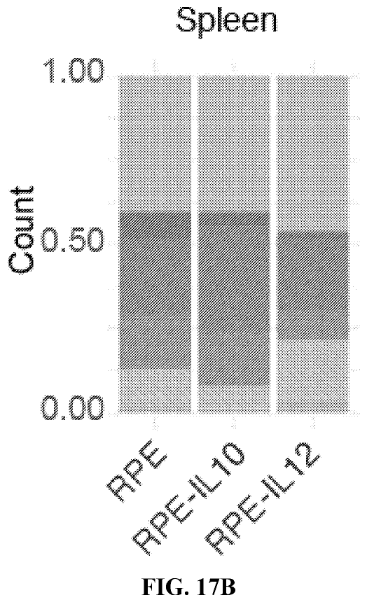

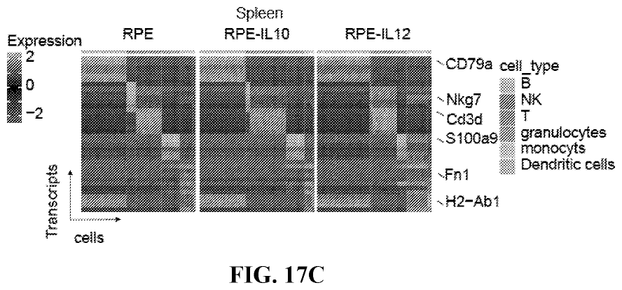

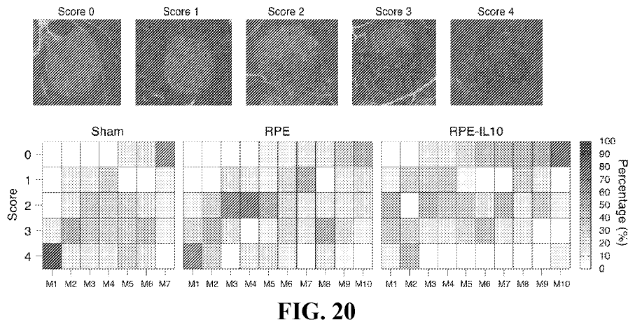

Finally, the systematic effect of cytokines on the immune composition in the spleen in RPE-IL10 or RPE-IL12 groups compared to RPE. (FIG. 17B-C). RPE-IL10 does not significantly affect the gene expression related to inflammatory gene sets, including allograft rejection (NES=−1.21, p=0.13, FIG. 17) and antigen processing cross-presentation (NES=−1.01, p=0.43, FIG. 17), indicating the effect of RPE-IL10 is primarily localized. In contrast, RPE-IL12 significantly promoted inflammatory response, including allograft rejection (NES=1.64, p=7.8e-4) and antigen processing cross-presentation (NES=2.04, p=6.9e-6, FIG. 20), and suppress TGFβ pathway (NES=−1.96, p=5.8e-4, FIG. 20) in splenic monocytes, indicating the effect of RPE-IL12 is systematic. Therefore RPE-IL10 may be a candidate cytokine to prevent fibrosis with minimal systematic effect on the host's immune cells.

### Example 6. IL-10 Secreting Implantable Constructs with Xenogeneic Human Islets Enable Long-Term Normal Glycemic Control in an Immunocompetent Diabetic Animal Model

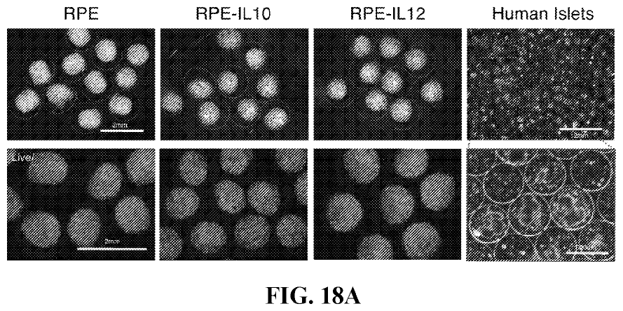

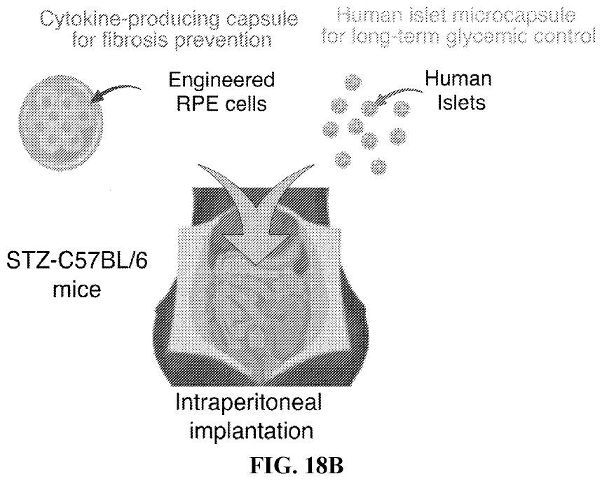

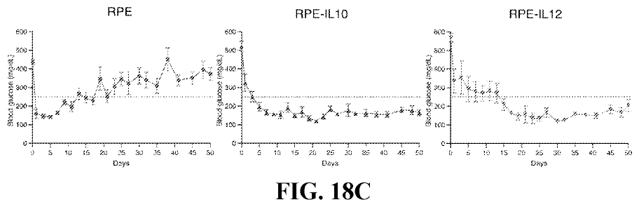

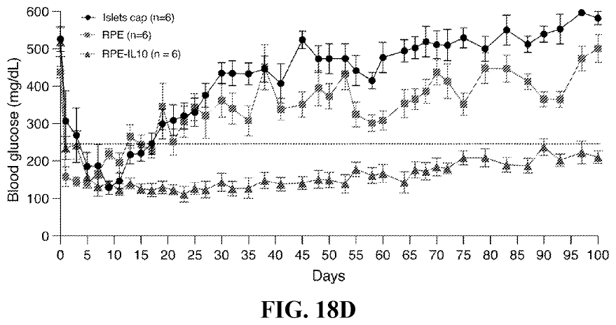

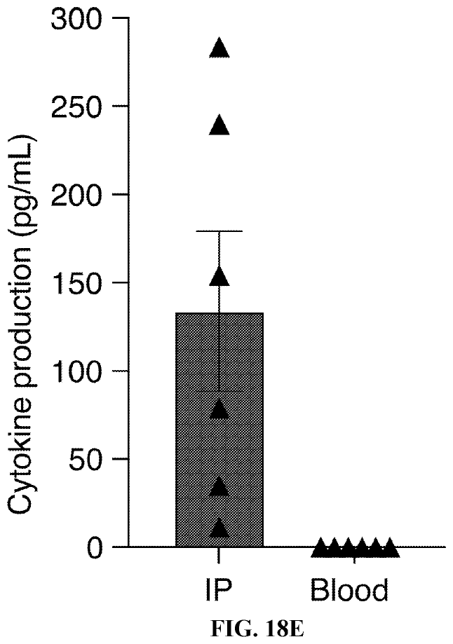

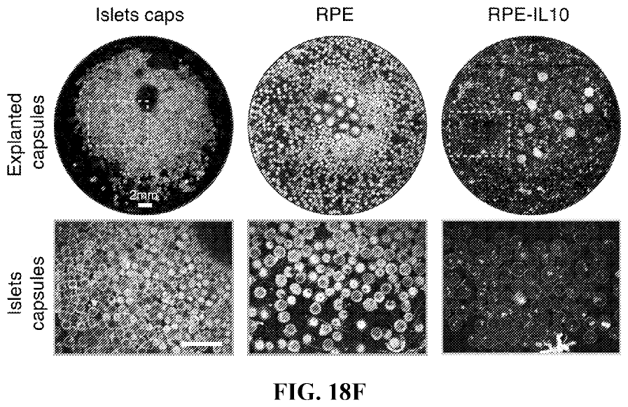

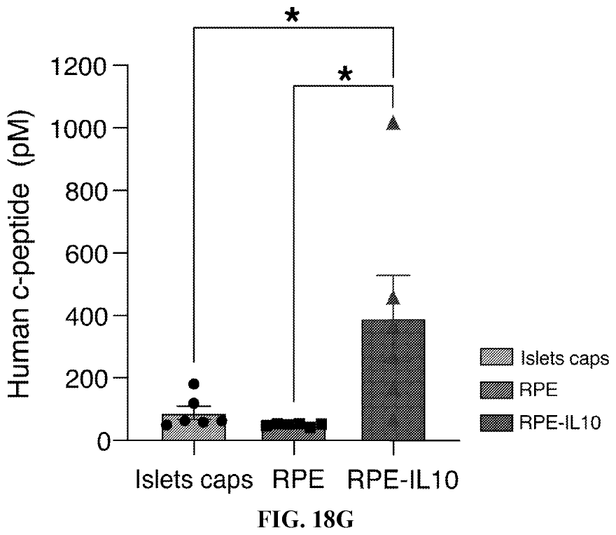

The example set forth below characterizes the efficacy of RPE-IL10 and RPE-IL12 implantable constructs for enabling long-term viability and protection of xenograft islets. In short, implantable constructs comprising cells capable of expressing IL-10 (RPE-IL10) and IL-12 (RPE-IL12), respectively, were implanted in the intraperitoneal (IP) space in streptozotocin (STZ)-induced diabetic C57BL/6J mice together with encapsulated human islets as shown in FIGS. 18A-B. RPE, RPE-IL10, and RPE-IL12 cells were encapsulated in 1.5 mm-diamter alginate implantable constructs as previously described at a cell density of 10,000 cells per implantable construct. 10 implantable constructs were implanted in each mouse specimen for a total cell count of 100,000 cells/mouse. Empty implantable constructs used in the fibrosis study as described in Example 3 were substituted with encapsulated human islets. 2,000 islet equivalents (IEQ) were co-delivered in the IP space. As with the fibrosis study of Example 3, islet implantable constructs were fabricated at 0.5-mm diameter in order to trigger a more robust immune response upon implantation. Nonengineered RPE implantable constructs co-delivered with the islet implantable constructs were designated as the negative control group. Upon implantation, BG concentrations under non-fasting conditions were drastically decreased and restored to a normal glycemic levels in all groups. However, the RPE group failed glycemic correction at week three (FIG. 18C). On the other hand, RPE-IL10 and RPE-IL12 groups demonstrated constant BG controls in diabetic mice for up to 50 days with average BG levels below 250 mg/dL, the normoglycemic concentration (FIG. 18C).

In order to evaluate the longevity of BG correction caused by the cytokine-secreting implantable constructs, BG concentrations of the RPE-IL10 group were monitored for 100 days in C57BL/6J diabetic mice. Encapsulated islets and implantable constructs containing RPE cells (RPE group), and only encapsulated islets (islet cap group) were designated as experimental controls. The RPE-IL12 group was not interrogated in this long-term study due to complications such as fluid accumulation under diabetic condition. The RPE-IL10 group demonstrated a robust and extended duration of BG regulation for 100 days relative to both the islet cap groups which both failed before three weeks as shown in FIG. 18D. IP fluid concentration of IL-10 demonstrated long-term production of IL-10 from implantable constructs even after 100 days in diabetic mice (FIG. 18E). Further, the dark-field microscopy images of explanted implantable constructs shows minimal fibrotic overgrowth on the surface of microimplantable constructs from the RPE-IL10 group relative to both the RPE and islet cap groups as shown in FIG. 18F. Additionally, the concentration of human c-peptide, a surrogate biomarker for insulin production, was measured from the serum separated from mouse blood 100 days after transplantation. Significantly higher levels of c-peptide secretion were detected in the RPE-IL10 group relative to the RPE and islet cap groups, suggesting improved long-term functionality of islets as shown in FIG. 18G. These data demonstrate that RPE-IL10 implantable constructs can protect encapsulated xenograft islets form the fibrotic response in an immunocompetent diabetic mouse model and enable longer viability and functionality of these grafts.

### Example 7. IL-10 Secreting Implantable Constructs Delay Onset of Diabetes in NOD Mice

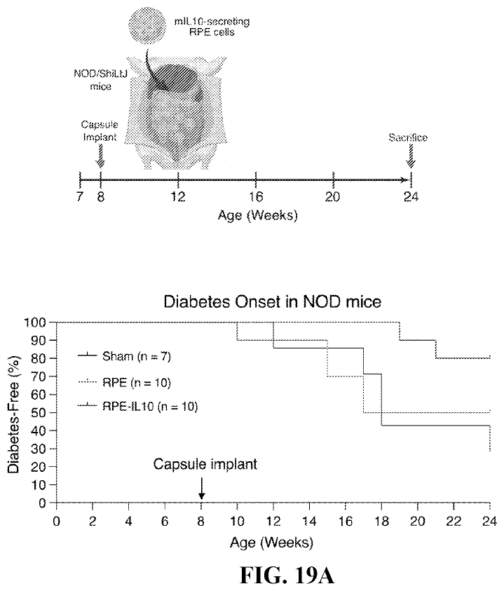

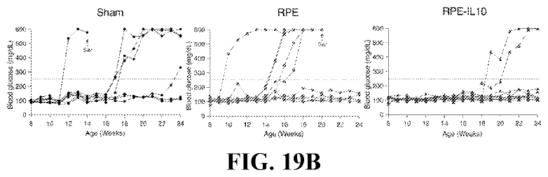

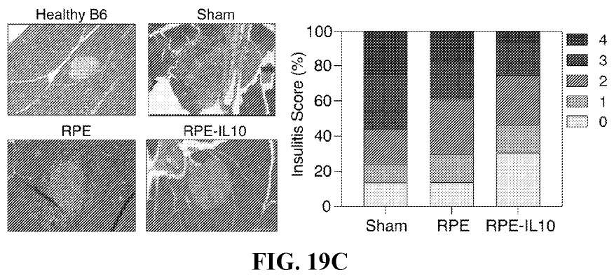

The example set forth below describes the protection afforded from the onset of type I diabetes from IL-10 secreting implantable constructs in a NOD mouse model. Briefly, 10 implantable constructs encapsulating RPE-IL10 cells were implanted into the IP space of non-obese pre-diabetic (NOD/ShiLtJ, NOD) mice at eight weeks old before they developed diabetes as shown in FIG. 19A. The NOD mouse is generally recognized as a gold standard model for studying TID, closely emulating human TID by experiencing insulitis through the accumulation of leukocytes and the infiltration of lymphocytes to islets. The indicators of pre-insulitis naturally advance to diabetes. Hence, utilization of this model can validate whether the cytokine-producing implantable constructs can mitigate islet destruction mediated by lymphocyte infiltration. All mice were sacrificed at 24 weeks old. NOD mice receiving implantable constructs comprising non-engineered cells (RPE group) served as a Control; mice in the sham group received 1 ml of saline. BG concentrations below 250 mg/dL were considered as healthy and contraindicative of diabetes. At 24 weeks of age (16 weeks after implantation), half of the RPE group and 70% of the sham group developed diabetes (FIG. 19B). In contrast, results from the RPE-IL10 group indicated that it prevented diabetes onset in 80% of mice (FIG. 19B). Furthermore, the impact RPE-IL10 implantable constructs on islet inflammation was assessed via histology analysis of pancreases. For evaluating insulitis levels, at least five islets were scored from each mouse according to the following ranking: 0: no insulitis (i.e., no inflammation); 1: peri-insulitis (i.e., lymphocytes surrounding the islet; 2:25-50% of the islet infiltrated by lymphocytes; 3: infiltration between 50-70% of the islet; and 4: nearly complete or complete infiltration and destruction of the islet (FIG. 20). Sham and RPE groups had the majority of islets infiltrated by lymphocytes or islets almost completely destroyed (shown as a score of 3 or 4), while the RPE-IL10 group had the majority of islets showing no signs of insulitis (shown as a score of 0) as shown in FIGS. 19C and 20.

### Example 8. Encapsulated IL-35-Secreting Cells for Prevention of Onset of Diabetes

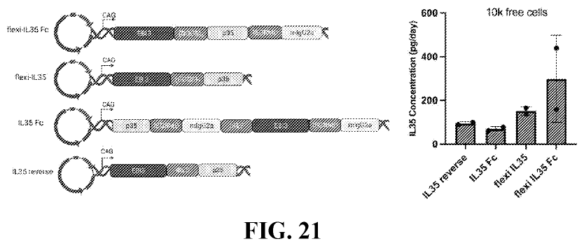

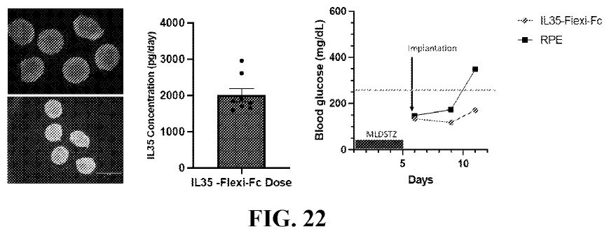

The example set forth below describes a protocol for the encapsulation of IL35-secreting cells in alginate implantable constructs for the prevention of onset of diabetes. Briefly, retinal pigmented epithelial (RPE) cells capable of secreting IL-35 were evaluated in an in vitro model. IL-35 was selected as accumulating evidence in the literature suggests that IL-35 plasma levels are decreased in patients with TID diabetes relative to health (non-diabetic) individuals. Moreover, recombinant IL-35 injection into the IP space of TID mouse models demonstrated prevention of onset as described in Example 7. A schematic of the IL-35 expression plasmids is shown in FIG. 21. CAG: CMV enhancer; EIBI3: Epstein Barr induced gene 3 (subunit of IL35); (GGGS) 3: glycine/serine flexible linker; p35: subunit of IL35; IEGRMD: linker peptide; mIgG2a: Fc region; IRES: internal ribosome entry site; P2A: self-cleaving peptide. Flexi-IL35 FC plasmid contained CAG, EBI3, (GGGS) 3, p35, IEGRMD, and mIgG2a; flexi-IL35 plasmid contained CAG, EBI3, (GGGS) 3, and p35; IL35 Fc plasmid contains CAG, p35, IEGRMD, mIgG2a, IRES, EBI3, IEGRMD, and mIgG21; and IL35 reverse plasmid contained CAG, EBI3, IRES, and p35. The secretion of IL35 was evaluated by ELISA from RPE cells transfected with each of the IL-35 expression plasmids. The media was changed 24 h after transfection. As shown in FIG. 21, encapsulated Flexi-IL35 FC cells were observed to have the highest secretion of IL-35 and were utilized for further in vivo experiments. As shown in FIG. 22, encapsulated Flexi-IL35 FC cells demonstrated prevention of onset of diabetes in mildly STZ-induced diabetic mice.

### Example 9. Validation of Cellular IL-12p35 Production

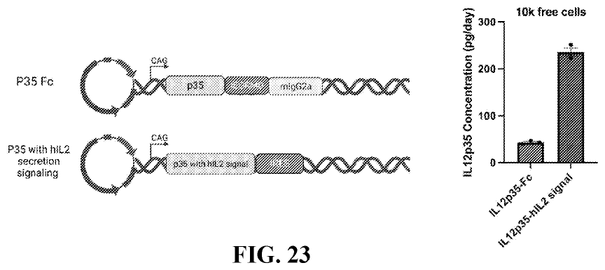

The example set forth below describes a protocol for the encapsulation of IL-12p35-secreting cells in alginate implantable constructs for the prevention of onset of diabetes. Briefly, retinal pigmented epithelial (RPE) cells capable of secreting IL-12p35 were evaluated in an in vitro model. A schematic of the IL-12p35 expression plasmids is shown in FIG. 23. CAG: CMV enhancer; IRES: internal ribosome entry site; IEGRMD: linker peptide; mIgG21: Fc region. P35 Fc plasmid contained CAG, p35, IEGRMD, and mIgG2a. P35 plasmid with hIL2 secretion signaling contained CAG, p35 with hIL2 signal, and IRES. The secretion of IL-12p35 was evaluated by ELISA from RPE cells transfected with each of the IL-12p35 expression plasmids. The media was changed 24 h after transfection. As evidenced from the bar graph in FIG. 23, the IL-12p35 expression plasmid with hIL2 signaling proved to be the superior expression vector and may prove useful for further in vivo experiments employing a rodent diabetes model.

### Example 10. Validation of Cellular IL-23 and IL-27 Production

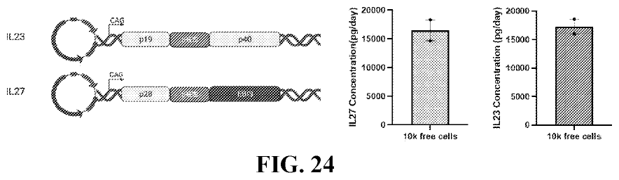

The example set forth below describes a protocol for the encapsulation of IL-23- and IL-27-secreting cells in alginate implantable constructs for the prevention of onset of diabetes. Briefly, retinal pigmented epithelial (RPE) cells capable of secreting IL-23 and IL-27 were evaluated in an in vitro model. A schematic of the IL-23 and IL-27 expression plasmids is shown in FIG. 24. CAG: CMV enhancer; IRES: internal ribosome entry site. IL23 plasmid contained CAG, p19, IRES, and p40; IL27 plasmid contained CAG, p28, IRES, and EBI3. The secretion of IL-23 and IL-27, respectively, was evaluated by ELISA from RPE cells transfected with the IL-23 and IL-27 expression plasmids. The media was changed 24 h after transfection. As shown in the bar graphs in FIG. 24, IL23 and IL27 production exceeded 15,000 μg/day for 10,000 free cells.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. An implantable construct comprising an engineered cell expressing one or more cytokines, such as interleukin-12 (IL-12), interleukin-10 (IL-10) and/or interleukin-35 (IL-35), such as heterodimeric IL-12.

2. The implantable construct of claim 1, wherein expression of said one or more cytokines is continuous, optionally further regulatable, such downregulated by an inducible “off” control.

3. The implantable construct of claim 1 or 2, further comprising a therapeutic agent.

4. The method of claim 3, wherein said therapeutic agent is a DNA, RNA, or an oligonucleotide, a further modulator of a host immune system, an organo-pharmaceutical compound, a toxin, or a protein (other than IL-12, IL-10 or IL-35).

5. The implantable construct of claim 3, wherein the implantable construct provides sustained or pulsatile release of the therapeutic agent.

6. The implantable construct of claim 1 or 2, wherein the engineered cell is a Chinese hamster ovary (CHO) cell, a retinal pigment epithelial cell (ARPE-19), a human mammary epithelial cell (MCF-10a and MCF-7), a human embryonic kidney (HEK) cell, a mesenchymal stem cell (MSC), a human umbilical vein endothelial cell (HUVEC), an NIH/3T3 cell, a BJ fibroblast cell, or a human renal mix epithelial cell (HREC).

7. The implantable construct of claim 3, wherein the therapeutic agent is released over at least 1 hour to 30 days, 60 days, 90 days, 180 days or 1 year.

8. The implantable construct of claim 1 or 2, wherein the implantable construct comprises a polymeric hydrogel.

9. The implantable construct of claim 8, wherein the polymeric hydrogel comprises chitosan, cellulose, hyaluronic acid, or alginate.

10. A method of providing an implantable construct to a subject comprising implanting into to the subject, or providing the subject with, an implantable construct according to any one of claims 1-9.

11. A method of making or manufacturing an implantable construct comprising introducing into an implantable construct an engineered cell expressing one or more cytokines, such as interleukin-12 (IL-12), interleukin-10 (IL-10) and/or interleukin-35 (IL-35).

12. The method of claim 11, wherein expression of said one or more cytokines is regulatable, such as by an inducible “off” control.

13. The method of claim 11 or 12, wherein the implantable construct further comprises a therapeutic agent.

14. The method of claim 11 or 12, wherein the engineered cell is a Chinese hamster ovary (CHO) cell, a retinal pigment epithelial cell (ARPE-19), a human mammary epithelial cell (MCF-10a and MCF-7), a human embryonic kidney (HEK) cell, a mesenchymal stem cell (MSC), a human umbilical vein endothelial cell (HUVEC), an NIH/3T3 cell, a BJ fibroblast cell, or a human renal mix epithelial cell (HREC).

15. The method of claim 11 or 12, wherein the implantable construct comprises a polymeric hydrogel, such as chitosan, cellulose, hyaluronic acid, or alginate.


SEQUENCE LISTING (13 sequences)

SEQ ID NO: 1 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 2 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 3 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 4 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 6 - DNA, 21 bp, synthetic construct
  source 1..21: mol_type = other DNA
cgcttccgctgcccagagact

SEQ ID NO: 7 - DNA, 26 bp, synthetic construct
  source 1..26: mol_type = other DNA
tataggtggtttcgtggatgcccgct

SEQ ID NO: 8 - DNA, 21 bp, synthetic construct
  source 1..21: mol_type = other DNA
catgttcagctttgtggacct

SEQ ID NO: 9 - DNA, 20 bp, synthetic construct
  source 1..20: mol_type = other DNA
gcagctgacttcagggatgt

SEQ ID NO: 10 - DNA, 22 bp, synthetic construct
  source 1..22: mol_type = other DNA
gcaggttcacctactctgtcct

SEQ ID NO: 11 - DNA, 20 bp, synthetic construct
  source 1..20: mol_type = other DNA
cttgccccattcatttgtct

SEQ ID NO: 12 - DNA, 22 bp, synthetic construct
  source 1..22: mol_type = other DNA
gcttctttgcagctccttcgtt

SEQ ID NO: 13 - DNA, 20 bp, synthetic construct
  source 1..20: mol_type = other DNA
cggagccgttgtcgacgacc